SEQUENCE LISTING (38 sequences)

SEQ ID NO: 1 - protein, 23 aa, Artificial Sequence
  Synthetic peptide
DIQMTQSPSSLSASVGDRVTITC

SEQ ID NO: 2 - protein, 15 aa, Artificial Sequence
  Synthetic peptide
WYQQKPGKAPKLLIY

SEQ ID NO: 3 - protein, 32 aa, Artificial Sequence
  Synthetic polypeptide
GVPSRFSGSGSGTDFTLTISSLQPEDFATYYC

SEQ ID NO: 4 - protein, 11 aa, Artificial Sequence
  Synthetic peptide
FGQGTKVEIKR

SEQ ID NO: 5 - protein, 17 aa, Artificial Sequence
  Synthetic peptide
KSSQSLLYTSSQKNYLA

SEQ ID NO: 6 - protein, 7 aa, Artificial Sequence
  Synthetic peptide
WASTRES

SEQ ID NO: 7 - protein, 9 aa, Artificial Sequence
  Synthetic peptide
QQYYAYPWT

SEQ ID NO: 8 - protein, 106 aa, Artificial Sequence
  Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 25 aa, Artificial Sequence [sequence omitted]

SEQ ID NO: 10 - protein, 13 aa, Artificial Sequence
  Synthetic peptide
WVRQAPGKGLEWV

SEQ ID NO: 11 - protein, 30 aa, Artificial Sequence
  Synthetic polypeptide
RFTISADTSKNTAYLQMNSLRAEDTAVYYC

SEQ ID NO: 12 - protein, 11 aa, Artificial Sequence
  Synthetic peptide
WGQGTLVTVSS

SEQ ID NO: 13 - protein, 10 aa, Artificial Sequence
  Synthetic peptide
GYTFTSYWLH

SEQ ID NO: 14 - protein, 18 aa, Artificial Sequence
  Synthetic peptide
GMIDPSNSDTRFNPNFKD

SEQ ID NO: 15 - protein, 12 aa, Artificial Sequence
  Synthetic peptide
ATYRSYVTPLDY

SEQ ID NO: 16 - protein, 108 aa, Artificial Sequence
  Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 17 - protein, 222 aa, Artificial Sequence
  Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 222 aa, Artificial Sequence
  Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 119 aa, Artificial Sequence
  Synthetic polypeptide
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSS

SEQ ID NO: 20 - protein, 113 aa, Artificial Sequence
  Synthetic polypeptide
DIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIK

SEQ ID NO: 21 - protein, 11 aa, Artificial Sequence
  Synthetic peptide
DICLPRWGCLW

SEQ ID NO: 22 - protein, 5 aa, Artificial Sequence
  Synthetic peptide
LDAQT

SEQ ID NO: 23 - protein, 9 aa, Artificial Sequence
  Synthetic peptide
LTEKRKKRS

SEQ ID NO: 24 - protein, 8 aa, Artificial Sequence
  Synthetic peptide
KPDSAEPM

SEQ ID NO: 25 - protein, 8 aa, Artificial Sequence
  Synthetic peptide
NVRCLQHF

SEQ ID NO: 26 - DNA, 23 bp, Artificial Sequence
  Synthetic primer
accccaatgagaccaatgaaatc

SEQ ID NO: 27 - DNA, 23 bp, Artificial Sequence
  Synthetic primer
atctttgatgagcttccggatct

SEQ ID NO: 28 - DNA, 18 bp, Artificial Sequence
  Synthetic probe
aatgccaactcccgtcag

SEQ ID NO: 29 - DNA, 28 bp, Artificial Sequence
  Synthetic primer
cattaaaggagacctcaccatagctaat

SEQ ID NO: 30 - DNA, 22 bp, Artificial Sequence
  Synthetic primer
cctgatcgagaaaccacaacct

SEQ ID NO: 31 - DNA, 25 bp, Artificial Sequence
  Synthetic probe
catgaagcgaccctctgatgtccca

SEQ ID NO: 32 - DNA, 65 bp, Artificial Sequence
  Synthetic oligonucleotide
gatccccgaacagaatcactgacatattcaagagatatgtcagtgattctgttcttttttggaaa

SEQ ID NO: 33 - DNA, 65 bp, Artificial Sequence
  Synthetic oligonucleotide
gatccccgaaactgtatgctggatgattcaagagatcatccagcatacagtttcttttttggaaa

SEQ ID NO: 34 - DNA, 67 bp, Artificial Sequence
  Synthetic oligonucleotide
gatccccagatccgccacaacatcgattcaagagatcgatgttgtggcggatcttgttttttggaaa

SEQ ID NO: 35 - protein, 449 aa, Artificial Sequence
  Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 220 aa, Artificial Sequence
  Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 119 aa, Artificial Sequence [sequence omitted]

SEQ ID NO: 38 - protein, 114 aa, Artificial Sequence
  Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

(19) United States
(12) Patent Application Publication
(10) Pub. No.: US 2016/0303127 A1
(43) Pub. Date: Oct. 20, 2016

(54) COMBINATION THERAPY WITH C-MET AND EGFR ANTAGONISTS

(71) Applicant: Genentech, Inc., South San Francisco, CA (US)

(72) Inventors: Ellen FILVAROFF, San Francisco, CA (US); Mark MERCHANT, Belmont, CA (US); Robert L. YAUCH, Redwood City, CA (US)

(21) Appl. No.: 14/973,459

(22) Filed: Dec. 17, 2015

Related U.S. Application Data
(63) Continuation of application No. 14/198,359, filed on Mar. 5, 2014, now abandoned.
Application No. 14/198,359 is a continuation of application No. 12/399,866, filed on Mar. 6, 2009, now abandoned.
(60) Provisional application No. 61/044,438, filed on Apr. 11, 2008.
(60) Provisional application No. 61/034,446, filed on Mar. 6, 2008.

(51) Int. Cl. A61K 31/517; A61K 45/06; A61K 31/5377; A61K 39/395

(52) U.S. Cl. CPC A61K 31/517; A61K 39/39558; A61K 45/06; A61K 31/5377; A61K 2039/505

(57) ABSTRACT

The present invention relates generally to the fields of molecular biology and growth factor regulation. More specifically, the invention relates to combination therapies for the treatment of pathological conditions, such as cancer.

### CROSS-REFERENCE TO RELATED APPLICATIONS

This application is a continuation of U.S. patent application Ser. No. 14/198,359, filed Mar. 5, 2014, which is a continuation application of U.S. patent application Ser. No. 12/399,866, filed Mar. 6, 2009, which claims priority under 35 USC §119(e) to U.S. provisional application No. 61/034,446, filed Mar. 6, 2008, and U.S. provisional application No. 61/044,438, filed Apr. 11, 2008, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392014602SeqList.txt, date recorded: Mar. 7, 2016, size: 23 KB).

### TECHNICAL FIELD

The present invention relates generally to the fields of molecular biology and growth factor regulation. More specifically, the invention relates to combination therapies for the treatment of pathological conditions, such as cancer.

### BACKGROUND

HGF is a mesenchyme-derived pleiotrophic factor with mitogenic, motogenic and morphogenic activities on a number of different cell types. HGF effects are mediated through a specific tyrosine kinase, c-met, and aberrant HGF and c-met expression are frequently observed in a variety of tumors. See, e.g., Maulik et al., Cytokine & Growth Factor Reviews (2002), 13:41-59; Danilkovitch-Miagkova & Zbar, J. Clin. Invest. (2002), 109(7):863-867. Regulation of the HGF/c-Met signaling pathway is implicated in tumor progression and metastasis. See, e.g., Trusolino & Comoglio, Nature Rev. (2002), 2:289-300).

HGF binds the extracellular domain of the c-met receptor tyrosine kinase (RTK) and regulates diverse biological processes such as cell scattering, proliferation, and survival. HGF-Met signaling is essential for normal embryonic development especially in migration of muscle progenitor cells and development of the liver and nervous system (Bladt et al., Nature (1995), 376, 768-771.; Hamanoue et al., Faseb J (2000), 14, 399-406; Maina et al., Cell (1996), 87, 531-542; Schmidt et al., Nature (1995), 373, 699-702; Uehara et al., Nature (1995), 373, 702-705). Developmental phenotypes of Met and HGF knockout mice are very similar suggesting that HGF is the cognate ligand for the Met receptor (Schmidt et al., 1995, supra; Uehara et al., 1995, supra). HGF-Met also plays a role in liver regeneration, angiogenesis, and wound healing (Bussolino et al., J Cell Biol (1992), 119, 629-641; Matsumoto and Nakamura, Exs (1993), 65, 225-249; Nusrat et al., J Clin Invest (1994) 93, 2056-2065). The precursor Met receptor undergoes proteolytic cleavage into an extracellular α subunit and membrane spanning β subunit linked by disulfide bonds (Tempest et al., Br J Cancer (1988), 58, 3-7). The β subunit contains the cytoplasmic kinase domain and harbors a multi-substrate docking site at the C-terminus where adapter proteins bind and initiate signaling (Bardelli et al., Oncogene (1997), 15, 3103-3111; Nguyen et al., J Biol Chem (1997), 272, 20811-20819; Pelicci et al., Oncogene (1995), 10, 1631-1638; Ponzetto et al., Cell (1994), 77, 261-271; Weidner et al., Nature (1996), 384, 173-176). Upon HGF binding, activation of Met leads to tyrosine phosphorylation and downstream signaling through Gab1 and Grb2/Sos mediated PI3-kinase and Ras/MAPK activation respectively, which drives cell motility and proliferation (Furge et al., Oncogene (2000), 19, 5582-5589; Hartmann et al., J Biol Chem (1994), 269, 21936-21939; Ponzetto et al., J Biol Chem (1996), 271, 14119-14123; Royal and Park, J Biol Chem (1995), 270, 27780-27787).

Met was shown to be transforming in a carcinogen-treated osteosarcoma cell line (Cooper et al., Nature (1984), 311, 29-33; Park et al., Cell (1986), 45, 895-904). Met overexpression or gene-amplification has been observed in a variety of human cancers. For example, Met protein is overexpressed at least 5-fold in colorectal cancers and reported to be gene-amplified in liver metastasis (Di Renzo et al., Clin Cancer Res (1995), 1, 147-154; Liu et al., Oncogene (1992), 7, 181-185). Met protein is also reported to be overexpressed in oral squamous cell carcinoma, hepatocellular carcinoma, renal cell carcinoma, breast carcinoma, and lung carcinoma (Jin et al., Cancer (1997), 79, 749-760; Morello et al., J Cell Physiol (2001), 189, 285-290; Natali et al., Int J Cancer (1996), 69, 212-217; Olivero et al., Br J Cancer (1996), 74, 1862-1868; Suzuki et al., Br J Cancer (1996), 74, 1862-1868). In addition, overexpression of mRNA has been observed in hepatocellular carcinoma, gastric carcinoma, and colorectal carcinoma (Boix et al., Hepatology (1994), 19, 88-91; Kuniyasu et al., Int J Cancer (1993), 55, 72-75; Liu et al., Oncogene (1992), 7, 181-185).

A number of mutations in the kinase domain of Met have been found in renal papillary carcinoma which leads to constitutive receptor activation (Olivero et al., Int J Cancer (1999), 82, 640-643; Schmidt et al., Nat Genet (1997), 16, 68-73; Schmidt et al., Oncogene (1999), 18, 2343-2350). These activating mutations confer constitutive Met tyrosine phosphorylation and result in MAPK activation, focus formation, and tumorigenesis (Jeffers et al., Proc Natl Acad Sci U S A (1997), 94, 11445-11450). In addition, these mutations enhance cell motility and invasion (Giordano et al., Faseb J (2000), 14, 399-406; Lorenzato et al., Cancer Res (2002), 62, 7025-7030). HGF-dependent Met activation in transformed cells mediates increased motility, scattering, and migration which eventually leads to invasive tumor growth and metastasis (Jeffers et al., Mol Cell Biol (1996), 16, 1115-1125; Meiners et al., Oncogene (1998), 16, 9-20).

Met has been shown to interact with other proteins that drive receptor activation, transformation, and invasion. In neoplastic cells, Met is reported to interact with α6β4 integrin, a receptor for extracellular matrix (ECM) components such as laminins, to promote HGF-dependent invasive growth (Trusolino et al., Cell (2001), 107, 643-654). In addition, the extracellular domain of Met has been shown to interact with a member of the semaphorin family, plexin B1, and to enhance invasive growth (Giordano et al., Nat Cell Biol (2002), 4, 720-724). Furthermore, CD44v6, which has been implicated in tumorigenesis and metastasis, is also reported to form a complex with Met and HGF and result in Met receptor activation (Orian-Rousseau et al., Genes Dev (2002), 16, 3074-3086).

Met is a member of the subfamily of receptor tyrosine kinases (RTKs) which include Ron and Sea (Maulik et al., Cytokine Growth Factor Rev (2002), 13, 41-59). Prediction of the extracellular domain structure of Met suggests shared homology with the semaphorins and plexins. The N-terminus of Met contains a Sema domain of approximately 500 amino acids that is conserved in all semaphorins and plexins. The semaphorins and plexins belong to a large family of secreted and membrane-bound proteins first described for their role in neural development (Van Vactor and Lorenz, Curr Bio (1999), 19, R201-204). However, more recently semaphorin overexpression has been correlated with tumor invasion and metastasis. A cysteine-rich PSI domain (also referred to as a Met Related Sequence domain) found in plexins, semaphorins, and integrins lies adjacent to the Sema domain followed by four IPT repeats that are immunoglobulin-like regions found in plexins and transcription factors. A recent study suggests that the Met Sema domain is sufficient for HGF and heparin binding (Gherardi et al., Proc Natl Acad Sci U S A (2003), 100(21):12039-44).

As noted above, the Met receptor tyrosine kinase is activated by its cognate ligand HGF and receptor phosphorylation activates downstream pathways of MAPK, PI-3 kinase and PLC-γ (L. Trusolino and P. M. Comoglio, Nat Rev Cancer 2, 289 (2002); C. Birchmeier et al., Nat Rev Mol Cell Biol 4, 915 (2003)). Phosphorylation of Y1234/Y1235 within the kinase domain is critical for Met kinase activation while Y1349 and Y1356 in the multisubstrate docking site are important for binding of src homology-2 (SH2), phosphotyrosine binding (PTB), and Met binding domain (MBD) proteins (C. Ponzetto et al., Cell 77, 261 (1994); K. M. Weidner et al., Nature 384, 173 (1996); G. Pelicci et al., Oncogene 10, 1631 (1995)) to mediate activation of downstream signaling pathways. An additional juxtamembrane phosphorylation site, Y1003, has been well characterized for its binding to the tyrosine kinase binding (TKB) domain of the Cbl E3-ligase (P. Peschard et al., Mol Cell 8, 995 (2001); P. Peschard, N. Ishiyama, T. Lin, S. Lipkowitz, M. Park, J Biol Chem 279, 29565 (2004)). Cbl binding is reported to drive endophilin-mediated receptor endocytosis, ubiquitination, and subsequent receptor degradation (A. Petrelli et al., Nature 416, 187 (2002)). This mechanism of receptor downregulation has been described previously in the EGFR family that also harbor a similar Cbl binding site (K. Shtiegman, Y. Yarden, Semin Cancer Biol 13, 29 (2003); M. D. Marmor, Y. Yarden, Oncogene 23, 2057 (2004); P. Peschard, M. Park, Cancer Cell 3, 519 (2003)). Dysregulation of Met and HGF have been reported in a variety of tumors. Ligand-driven Met activation has been observed in several cancers. Elevated serum and intra-tumoral HGF is observed in lung, breast cancer, and multiple myeloma (J. M. Siegfried et al., Ann Thorac Surg 66, 1915 (1998); P. C. Ma et al., Anticancer Res 23, 49 (2003); B. E. Elliott et al. Can J Physiol Pharmacol 80, 91 (2002); C. Seidel, et al, Med Oncol 15, 145 (1998)). Overexpression of Met and/or HGF, Met amplification or mutation has been reported in various cancers such as colorectal, lung, gastric, and kidney cancer and is thought to drive ligand-independent receptor activation (C. Birchmeier et al, Nat Rev Mol Cell Biol 4, 915 (2003); G. Maulik et al., Cytokine Growth Factor Rev 13, 41 (2002)). Additionally, inducible overexpression of Met in a liver mouse model gives rise to hepatocellular carcinoma demonstrating that receptor overexpression drives ligand independent tumorigenesis (R. Wang, et al, J Cell Biol 153, 1023 (2001)). The most compelling evidence implicating Met in cancer is reported in familial and sporadic renal papillary carcinoma (RPC) patients. Mutations in the kinase domain of Met that lead to constitutive activation of the receptor were identified as germline and somatic mutations in RPC (L. Schmidt et al., Nat Genet 16, 68 (1997)). Introduction of these mutations in transgenic mouse models leads to tumorigenesis and metastasis. (M. Jeffers et al., Proc Natl Acad Sci U S A 94, 11445 (1997)).

The epidermal growth factor receptor (EGFR) family comprises four closely related receptors (HER1/EGFR, HER2, HER3 and HER4) involved in cellular responses such as differentiation and proliferation. Over-expression of the EGFR kinase, or its ligand TGF-alpha, is frequently associated with many cancers, including breast, lung, colorectal, ovarian, renal cell, bladder, head and neck cancers, glioblastomas, and astrocytomas, and is believed to contribute to the malignant growth of these tumors. A specific deletion-mutation in the EGFR gene (EGFRvIII) has also been found to increase cellular tumorigenicity. Activation of EGFR stimulated signaling pathways promote multiple processes that are potentially cancer-promoting, e.g. proliferation, angiogenesis, cell motility and invasion, decreased apoptosis and induction of drug resistance. Increased HER1/EGFR expression is frequently linked to advanced disease, metastases and poor prognosis. For example, in NSCLC and gastric cancer, increased HER1/EGFR expression has been shown to correlate with a high metastatic rate, poor tumor differentiation and increased tumor proliferation.

Mutations which activate the receptor's intrinsic protein tyrosine kinase activity and/or increase downstream signaling have been observed in NSCLC and glioblastoma. However the role of mutations as a principle mechanism in conferring sensitivity to EGF receptor inhibitors, for example erlotinib (TARCEVA®) or gefitinib, has been controversial. Mutant forms of the full length EGF receptor has been reported to predict responsiveness to the EGF receptor tyrosine kinase inhibitor gefitinib (Paez, J. G. et al. (2004) Science 304:1497-1500; Lynch, T. J. et al. (2004) N. Engl. J. Med. 350:2129-2139). Cell culture studies have shown that cell lines which express such mutant forms of the EGF receptor (i.e. H3255) were more sensitive to growth inhibition by the EGF receptor tyrosine kinase inhibitor gefitinib, and that much higher concentrations of gefitinib was required to inhibit the rumor cell lines expressing wild type EGF receptor. These observations suggests that specific mutant forms of the EGF receptor may reflect a greater sensitivity to EGF receptor inhibitors, but do not identify a completely non-responsive phenotype.

The development for use as anti-tumor agents of compounds that directly inhibit the kinase activity of the EGFR, as well as antibodies that reduce EGFR kinase activity by blocking EGFR activation, are areas of intense research effort (de Bono J. S. and Rowinsky, E. K. (2002) Trends in Mol. Medicine 8:S19-S26; Dancey, J. and Sausville, E. A. (2003) Nature Rev. Drug Discovery 2:92-313). Several studies have demonstrated, disclosed, or suggested that some EGFR kinase inhibitors might improve tumor cell or neoplasia killing when used in combination with certain other anti-cancer or chemotherapeutic agents or treatments (e.g. Herbst, R. S. et al. (2001) Expert Opin. Biol. Ther. 1:719-732; Solomon, B. et al (2003) Int. J. Radiat. Oncol. Biol. Phys. 55:713-723; Krishnan, S. et al. (2003) Frontiers in Bioscience 8, e1-13; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther. 3:367-380; Bulgaru, A. M. et al. (2003) Expert Rev. Anticancer Ther. 3:269-279; Dancey, J. and Sausville, E. A. (2003) Nature Rev. Drug Discovery 2:92-313; Ciardiello, F. et al. (2000) Clin. Cancer Res. 6:2053-2063; and Patent Publication No: US 2003/0157104).

Erlotinib (e.g. erlotinib HCl, also known as TARCEVA® or OSI-774) is an orally available inhibitor of EGFR kinase. In vitro, erlotinib has demonstrated substantial inhibitory activity against EGFR kinase in a number of human tumor cell lines, including colorectal and breast cancer (Moyer J. D. et al. (1997) Cancer Res. 57:4838), and preclinical evaluation has demonstrated activity against a number of EGFR-expressing human tumor xenografts (Pollack, V. A. et al (1999) J. Pharmacol. Exp. Ther. 291:739). Erlotinib has demonstrated activity in clinical trials in a number of indications, including head and neck cancer (Soulieres, D., et al. (2004) J. Clin. Oncol. 22:77), NSCLC (Perez-Soler R, et al. (2001) Proc. Am. Soc. Clin. Oncol. 20:310a, abstract 1235), CRC (Oza, M., et al. (2003) Proc. Am. Soc. Clin. Oncol. 22:196a, abstract 785) and MBC (Winer, E., et al. (2002) Breast Cancer Res. Treat. 76:5115a, abstract 445; Jones, R. J., et al. (2003) Proc. Am. Soc. Clin. Oncol. 22:45a, abstract 180). In a phase III trial, erlotinib monotherapy significantly prolonged survival, delayed disease progression and delayed worsening of lung cancer-related symptoms in patients with advanced, treatment-refractory NSCLC (Shepherd, F. et al. (2004) J. Clin. Oncology, 22:14S (July 15 Supplement), Abstract 7022). In November 2004 the U.S. Food and Drug Administration (FDA) approved TARCEVA® for the treatment of patients with locally advanced or metastatic non-small cell lung cancer (NSCLC) after failure of at least one prior chemotherapy regimen.

Publications relating to c-met and c-met antagonists include Martens, T, et al (2006) Clin Cancer Res 12(20 Pt 1):6144; U.S. Pat. No. 6,468,529; WO2006/015371; WO2007/063816; WO2006/104912; WO2006/104911; WO2006/113767; US2006-0270594; US2006-0270594; US2006-0293235; U.S. Pat. No. 7,481,993; WO2009/007427; WO2005/016382; WO2009/002521; WO2007/143098; WO2007/115049; WO2007/126799. Combination therapies with met antagonist and HER antagonists are described in co-owned, co-pending U.S. patent application Ser. No. ______ , filed Mar. 6, 2009, and U.S. Ser. Nos. 60/034,453, filed Mar. 6, 2008 and 61/044,433, filed Apr. 11, 2008.

Despite the significant advancement in the treatment of cancer, improved therapies are still being sought.

All references cited herein, including patent applications and publications, are incorporated by reference in their entirety.

### SUMMARY OF THE INVENTION

The present invention provides combination therapies for treating a pathological condition, such as cancer, wherein a c-met antagonist is combined with an EGFR antagonist, thereby providing significant anti-tumor activity.

In one aspect, the invention provides methods of treating a cancer in a subject, comprising administering to the subject a therapeutically effective amount of a c-met antagonist and an EGFR antagonist.

Examples of c-met antagonists include, but are not limited to, soluble c-met receptors, soluble HGF variants, apatmers or peptibodies that are specific to c-met or HGF, c-met small molecules, anti-c-met antibodies and anti-HGF antibodies. In some embodiment, the c-met antagonist is an anti-c-met antibody.

In some embodiments, the anti-c-met antibody comprises a heavy chain variable domain comprising one or more of CDR1-HC, CDR2-HC and CDR3-HC sequence depicted in FIG. 7 (SEQ ID NO: 13-15). In some embodiments, the antibody comprises a light chain variable domain comprising one or more of CDR1-LC, CDR2-LC and CDR3-LC sequence depicted in FIG. 7 (SEQ ID NO: 5-7). In some embodiments, the heavy chain variable domain comprises FR1-HC, FR2-HC, FR3-HC and FR4-HC sequence depicted in FIG. 7 (SEQ ID NO: 9-12). In some embodiments, the light chain variable domain comprises FR1-LC, FR2-LC, FR3-LC and FR4-LC sequence depicted in FIG. 7 (SEQ ID NO: 1-4). In some embodiments, the anti-cmet antibody is monovalent and comprises an Fc region. In some embodiments, the antibody comprises Fc sequence depicted in FIG. 7 (SEQ ID NO: 17).

In some embodiments, the antibody is monovalent and comprises a Fc region, wherein the Fc region comprises a first and a second polypeptide, wherein the first polypeptide comprises the Fc sequence depicted in FIG. 7 (SEQ ID NO: 17) and the second polypeptide comprises the Fc sequence depicted in FIG. 8 (SEQ ID NO: 18).

In one embodiment, the anti-c-met antibody comprises (a) a first polypeptide comprising a heavy chain variable domain having the sequence: QVQLQQSGPELVRPGASVKMSCRASGYTFTSYWLHWVKQRPGQGLEWIGMIDPSNSDTRFN PNFKDKATLNVDRSSNTAYMLLSSLTSADSAVYYCATYGSYVSPLDYWGQGTSVTVSS (SEQ ID NO:19), CH1 sequence depicted in FIG. 7 (SEQ ID NO: 16), and the Fc sequence depicted in FIG. 7 (SEQ NO: 17); and (b) a second polypeptide comprising a light chain variable domain having the sequence: DIMMSQSPSSLTVSVGEKVTVSCKSSQSLLYTSSQKNYLAWYQQKPGQSPKLLIYWASTRES GVPDRFTGSGSGTDFTLTITSVKADDLAVYYCQQYYAYPWTFGGGTKLEIK (SEQ ID NO:20), and CL1 sequence depicted in FIG. 7 (SEQ ID NO: 8); and (c) a third polypeptide comprising the Fc sequence depicted in FIG. 8 (SEQ ID NO: 18).

In one aspect, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816. For example, a hole mutation can be one or more of T366A, L368A and/or Y407V in an Fc polypeptide, and a cavity mutation can be T366W in an Fc polypeptide.

In some embodiments, the c-met antagonist is SGX-523, PF-02341066, JNJ-38877605, BMS-698769, PHA-665,752, SU5416, SU 1274, XL-880, MGCD265, ARQ 197, MP-470, AMG 102, antibody 223C4 or humanized antibody 223C4 (WO2009/007427), L2G7, NK4, XL-184, MP-470, or Comp-1.

C-met antagonists can be used to reduce or inhibit one or more aspects of HGF/c-met-associated effects, including but not limited to c-met activation, downstream molecular signaling (e.g., mitogen activated protein kinase (MAPK) phosphorylation, phosphorylation, c-met phosphorylation, PI3 kinase mediated signaling), cell proliferation, cell migration, cell survival, cell morphogenesis and angiogenesis. These effects can be modulated by any biologically relevant mechanism, including disruption of ligand (e.g., HGF) binding to c-met, disruption of c-met phosphorylation and/or disruption of c-met multimerization.

Examples of EGFR antagonists include antibodies and small molecules that bind to EGFR. EGFR antagonists also include small molecules such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, WO9935146, WO0132651, U.S. Pat. No. 6,344,455, U.S. Pat. No. 5,760,041, U.S. Pat. No. 6,002,008, U.S. Pat. No. 5,747,498. Particular small molecule EGFR antagonists include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa® (ZD1839, gefitinib, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide); lapatinib (Tykerb, GlaxoSmithKline); ZD6474 (Zactima, AstraZeneca); CUDC-101 (Curis); canertinib (CI-1033); AEE788 (6-[4-[(4-ethyl-1-piperazinyl)methyl]phenyl]-N-[(1R)-1-phenylethyl]-7H-pyrrolo[2,3-d]pyrimidin-4-amine, WO2003013541, Novartis) and PKI166 4-[4-[[(1R)-1-phenylethyl]amino]-7H-pyrrolo[2,3-d]pyrimidin-6-yl]-phenol, WO9702266 Novartis).

In a particular embodiment, the EGFR antagonist has a general formula I:

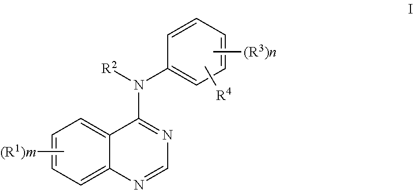

in accordance with U.S. Pat. No. 5,757,498, incorporated herein by reference, wherein:

m is 1, 2, or 3;

each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;

or each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶-piperazin-1-yl, imidazol-1-yl, 4-pyridon-1-yl, —(C₁-C₄ alkylene)(CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;

or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein said —NHSO₂R⁵, phthalimido—(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;

or two R¹ groups are taken together with the carbons to which they are attached to form a 5-8 membered ring that includes 1 or 2 heteroatoms selected from O, S and N;

R² is hydrogen or C₁-C₆ alkyl optionally substituted by 1 to 3 substituents independently selected from halo, C₁-C₄ alkoxy, —NR⁶R⁶, and —SO₂R⁵;

n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and

R⁴ is azido or -(ethynyl)-R¹¹ wherein R¹¹ is hydrogen or C₁-C₆ alkyl optionally substituted by hydroxy, —OR⁶, or —NR⁶R⁶.

In a particular embodiment, the EGFR antagonist is a compound according to formula I selected from the group consisting of:

(6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine; (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-{6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin-4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl]-(5-ethynyl-2-methyl-phenyl)-amine; [6,7-bis-(2-methoxy-ethoxy)-quinazolin-4-yl]-(3-ethynyl-4-fluoro-phenyl)-amine; [6,7-bis-(2-chloro-ethoxy)-quinazolin-4-yl]-(3-ethynyl-phenyl)-amine; [6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7-yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]-ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-[2-(4-methyl-piperazin-1-yl)-ethoxy]-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; 2-[4-(3-ethynyl-phenylamino)-6-(2-methoxy-ethoxy)-quinazolin-7-yloxy]-ethanol; (6,7-dipropoxy-quinazolin-4-yl)-(3-ethynyl-phenyl)-amine; (6,7-diethoxy-quinazolin4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine; (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; and (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine.

In a particular embodiment, the EGFR antagonist of formula I is N-(3-ethynylphenyl)-6,7-bis(2-methoxyethoxy)-4-quinazolinamine. In a particular embodiment, the EGFR antagonist N-(3-ethynylphenyl)-6,7-bis(2-methoxyethoxy)-4-quinazolinamine is in HCl salt form. In another particular embodiment, the EGFR antagonist N-(3-ethynylphenyl)-6,7-bis(2-methoxyethoxy)-4-quinazolinamine is in a substantially homogeneous crystalline polymorph form (described as polymorph B in WO 01/34,574) that exhibits an X-ray powder diffraction pattern having characteristic peaks expressed in degrees 2-theta at approximately 6.26, 12.48, 13.39, 16.96, 20.20, 21.10, 22.98, 24.46, 25.14 and 26.91. Such polymorph form of N-(3-ethynylphenyl)-6,7-bis(2-methoxyethoxy)-4-quinazolinamine is referred to as Tarceva® as well as OSI-774, CP-358774 and erlotinib.

EGFR antagonists can be used to reduce or inhibit one or more aspects of EGFR-EGFR ligand-associated effects, including but not limited to EGFR activation, downstream molecular signaling, cell proliferation. These effects can be modulated by any biologically relevant mechanism, including disruption of ligand binding to EGFR, and disruption of EGFR phosphorylation.

Methods of the invention can be used to affect any suitable pathological state. For example, methods of the invention can be used for treating different cancers, both solid tumors and soft-tissue tumors alike. Non-limiting examples of cancers amendable to the treatment of the invention include breast cancer, colorectal cancer, rectal cancer, non-small cell lung cancer (NSCLC), non-Hodgkins lymphoma (NHL), renal cell cancer, prostate cancer, liver cancer, pancreatic cancer, soft-tissue sarcoma, kaposi's sarcoma, sarcoma, renal cell carcinoma, carcinoid carcinoma, head and neck cancer, glioblastoma, melanoma, ovarian cancer, gastric cancer, mesothelioma, and multiple myeloma. In certain aspects, the cancers are metastatic. In other aspects, the cancers are non-metastatic.

In some embodiments, an anti-c-met antibody and erlotinib are used in combination therapies of cancers such as non-small cell lung carcinoma.

In certain embodiments, the cancer is not an EGFR antagonist (e.g., erlotinib or gefitinib) resistant cancer. In certain embodiments, the cancer is not an erlotinib or gefitinib resistant cancer.

In certain embodiments, the cancer is not a tyrosine kinase inhibitor-resistant cancer. In certain embodiments, the cancer is not a small molecule EGFR tyrosine kinase inhibitor-resistant cancer.

In certain embodiments, the cancer displays c-met and/or EGFR expression, amplification, or activation. In certain embodiments, the cancer does not display c-met and/or EGFR expression, amplification, or activation. In certain embodiments, the cancer displays c-met amplification. In certain embodiments, the cancer displays c-met amplification and EGFR amplification.

In certain embodiments, the cancer displays a wildtype EGFR gene. In certain embodiments, the cancer displays a wildtype EGFR gene and c-met amplification and/or c-met mutation.

In certain embodiments, the cancer displays EGFR mutation. Mutations can be located in any portion of an EGFR gene or regulatory region associated with an EGFR gene. Exemplary EGFR mutations include, for example, mutations in exon 18, 19, 20 or 21, mutations in the kinase domain, G719A, L858R, E746K, L747S, E749Q, A750P, A755V, V765M, S768I, L858P, E746-R748 del, R748-P753 del, M766-A767 AI ins, S768-V769 SVA ins, P772-H773 NS ins, 2402OC, 2482OA, 2486T>C, 2491 G>C, 2494OC, 251 0OT, 2539OA, 2549OT, 2563OT, 2819T>C, 2482-2490 del, 2486-2503 del, 2544-2545 ins GCCATA, 2554-2555 ins CCAGCGTGG, or 2562-2563 ins AACTCC. Other examples of EGFR activating mutations are known in the art (see e.g., US Patent Publication No. 2005/0272083). In certain embodiments, the cell or cell line does not comprise a T790M mutation in the EGFR gene.

In certain embodiments, the cancer displays c-met and/or EGFR activation. In certain embodiments, the cancer does not display c-met and/or EGFR activation.

In certain embodiments, the cancer displays constitutive c-met and/or EGFR activation. In some embodiments, the constitutively activated EGFR comprises a mutation in the tyrosine kinase domain. In certain embodiments, the cancer does not display constitutive c-met and/or EGFR activation.

In certain embodiments, the cancer displays ligand-independent c-met and/or EGFR activation. In certain embodiments, the cancer does not display ligand-independent c-met and/or EGFR activation.

In one aspect, the invention provides methods for treating a subject suffering from a cancer that is resistant to treatment with an ErbB antagonist, comprising administering to the subject an ErbB antagonist and a c-met antagonist.

In some embodiments, the cancer is lung cancer, brain cancer, breast cancer, head and neck cancer, colon cancer, ovarian cancer, gastric cancer, or pancreatic cancer. In some embodiments, the cancer is non-small cell lung cancer (NSCLC). In some embodiments, the subject has an EGFR, HER2, HER3, or HER4 activating mutation or gene amplification. In some embodiments, the subject has an EGFR activating mutation or an EGFR gene amplification. In some embodiments, the subject has a c-met activating mutation or a c-met gene amplification. In some embodiments, the cancer is resistant to treatment with one or more of the following ErbB antagonists: an EGFR antagonist, a HER2 antagonist, a HER3 antagonist, or a HER4 antagonist. In some embodiments, the cancer is resistant to treatment with one or more of the following ErbB antagonists: a small molecule therapeutic, a nucleic acid therapeutic, or a protein therapeutic. In some embodiments, the cancer is resistant to treatment with an anti-ErbB antibody. In some embodiments, the cancer is resistant to treatment with an siRNA targeted to an ErbB gene. In some embodiments, the cancer is resistant to treatment with an ErbB kinase inhibitor. In some embodiments, the cancer is resistant to treatment with an EGFR kinase inhibitor. In some embodiments, the cancer is resistant to treatment with one or more of the following EGFR antagonists: gefitinib, erlotinib, lapatinib, PF00299804, CI-1 033, EKB-569, BIBW2992, ZD6474, AV-412, EXEL-7647, HKI-272, cetuximab, pantinumumab, or trastuzumab. In some embodiments, one or more of the following ErbB antagonists is administered to the subject: an EGFR antagonist, a HER2 antagonist a HER3 antagonist, or a HER4 antagonist. In some embodiments, one or more of the following ErbB antagonists is administered to the subject: a small molecule therapeutic, a nucleic acid therapeutic, or a protein therapeutic. In some embodiments, one or more of the following EGFR antagonist is administered to the subject: gefitinib, erlotinib, lapatinib, PF00299804, CI-1033, EKB-569, BIBW2992, ZD6474, EXEL-7647, AV-412, HKI-272, cetuximab, pantinumumab, or trastuzumab. In some embodiments, one or more of the following c-met antagonists is administered to the subject: a small molecule therapeutic, a nucleic acid therapeutic, or a protein therapeutic. In some embodiments, one or more of the following c-met antagonists is administered to the subject: PHA-665,752, SU 1274, SU5416, PF-02341066, XL-880, MGCD265, XL184, ARQ 197, MP-470, SGX-523, JNJ38877605, AMG 102, or MetMAb. In some embodiments, the ErbB antagonist and the c-met antagonist are administered simultaneously to the subject. In some embodiments, the ErbB antagonist and the c-met antagonist are administered to the subject as a coformulation. In some embodiments, the methods of the invention further comprise administering at least one additional treatment to said subject. In some embodiments, the additional treatment is one or more of the following: administration of an additional therapeutic agent, radiation, photodynamic therapy, laser therapy, or surgery. In some embodiments, the subject is a mammal. In some embodiments, the mammal is a human.

In one aspect, the invention provides methods for treating a subject suffering from a cancer associated with an ErbB activating mutation or an ErbB gene amplification, wherein the subject has developed a resistance to treatment with an ErbB antagonist, comprising determining whether the subject has a c-met activating mutation or a c-met gene amplification, and administering to those subjects having a c-met activating mutation or a c-met gene amplification an ErbB antagonist and a c-met antagonist.

In one aspect, the invention provides methods for treating a subject suffering from a cancer associated with an ErbB activating mutation or an ErbB gene amplification, comprising: (i) monitoring a subject being treated with an ErbB antagonist to determine if the subject develops a c-met activating mutation or a c-met gene amplification, and (ii) modifying the treatment regimen of the subject to include a c-met antagonist in addition to the ErbB antagonist where the subject has developed a c-met activating mutation or a c-met gene amplification.

In one aspect, the invention provides methods for treating a subject suffering from a cancer associated with an ErbB activating mutation or an ErbB gene amplification, comprising: (i) monitoring a subject being treated with ErbB antagonist to determine if the subject develops a resistance to the inhibitor, (ii) testing the subject to determine whether the subject has a c-met activating mutation or a c-met gene amplification, and (iii) modifying the treatment regimen of the subject to include a c-met antagonist in addition to the ErbB antagonist where the subject has a c-met activating mutation or a c-met gene amplification.

In one aspect, the invention provides methods for evaluating ErbB antagonist, comprising: (i) monitoring a population of subjects being treated with an ErbB antagonist to identify those subjects that develop a resistance to the therapeutic, (ii) testing the resistant subjects to determine whether the subjects have a c-met activating mutation or a c-met gene amplification, and (iii) modifying the treatment regimen of the subjects to include a c-met antagonist in addition to the ErbB antagonist where the subjects have a c-met activating mutation or a c-met gene amplification.

In one aspect, the invention provides methods for reducing ErbB phosphorylation in a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for reducing PI3K mediated signaling in a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for reducing ErbB-mediated signaling in a cancer cell, wherein said cancer cell has acquired resistance to an ErbB antagonist, and wherein said cell comprises a c-met activating mutation or a c-met gene amplification, comprising contacting the cell with a c-met antagonist and an ErbB antagonist.

In one aspect, the invention provides methods for restoring sensitivity of a cancer cell to an ErbB antagonist, wherein said cancer cell has acquired resistance to an ErbB antagonist, and wherein said cell comprises a c-met activating mutation or a c-met gene amplification, comprising contacting the cell with a c-met antagonist and an ErbB antagonist.

In one aspect, the invention provides methods for reducing growth or proliferation of a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for increasing apoptosis of a cancer cell, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for reducing resistance of a cancer cell to an ErbB antagonist, wherein said cancer cell has acquired resistance to an ErbB antagonist, and wherein said cell comprises a c-met activating mutation or a c-met gene amplification, comprising the step of contacting the cell with a c-met antagonist and an ErbB antagonist.

In one aspect, the invention provides methods for treating acquired ErbB antagonist resistance in a cancer cell, wherein said cell comprises a c-met activating mutation or a c-met gene amplification, comprising contacting the cell with a c-met antagonist and an ErbB antagonist.

In some embodiments, the cancer cell is a mammalian cancer cell. In some embodiments, the mammalian cancer cell is a human cancer cell. In some embodiments, the cancer cell is a cell line. In some embodiments, the cancer cell is from a primary tissue sample. In some embodiments, the cancer cell is selected from the group consisting of: a lung cancer cell, a brain cancer cell, a breast cancer cell, a head and neck cancer cell, a colon cancer cell, an ovarian cancer cell, a gastric cancer cell or a pancreatic cancer cell. In some embodiments, the cancer cell is any ErbB-driven cancer. In some embodiments, the cancer cell comprises an ErbB activating mutation. In some embodiments, the ErbB activating mutation is an EGFR activating mutation. In some embodiments, the cancer cell comprises an ErbB gene amplification. In some embodiments, the ErbB gene amplification is an EGFR gene amplification. In some embodiments, the ErbB gene amplification is at least 2-fold. In some embodiments, the c-met amplification is at least 2-fold. In some embodiments, the cancer cell comprises an ErbB gene mutation associated with increased resistance to an ErbB antagonist. In some embodiments, the ErbB gene mutation associated with increased resistance to an ErbB antagonist is a T790M mutation of EGFR. In some embodiments, the ErbB antagonist is selected from the group consisting of: an EGFR antagonist, an HER2 antagonist an HER3 antagonist, or an HER4 antagonist. In some embodiments, the ErbB antagonist is a small molecule therapeutic, a nucleic acid therapeutic, or a protein therapeutic. In some embodiments, the ErbB antagonist is an antibody, an antisense molecule, or a small molecule kinase inhibitor. In some embodiments, the ErbB antagonist is an EGFR kinase inhibitor selected from the group consisting of: gefitinib, erlotinib, lapatinib, PF00299804, CI-1 033, EKB-569, BIBW2992, ZD6474, AV-412, HKI-272, EXEL-7647, cetuximab, pantinumumab, or trastuzumab. In some embodiments, the antibody is an anti-EGFR antibody selected from the group consisting of: cetuximab, pantinumumab, and trastuzumab. In some embodiments, the nucleic acid therapeutic is an siRNA molecule. In some embodiments, the c-met antagonist is a small molecule therapeutic, a nucleic acid therapeutic, or a protein therapeutic. In some embodiments, the c-met antagonist is an antibody directed against c-met or antibody directed against hepatocyte growth factor (HGF). In some embodiments, the c-met antagonist is PHA-665,752, SUI1274, SU5416, PF-02341066, XL-880, MGCD265, XL184, ARQ 197, MP-470, SGX-523, JNJ38877605, AMG 102, or MetMAb. In some embodiments, the nucleic acid therapeutic is an siRNA molecule. In some embodiments, the step of contacting said cell with a c-met antagonist and an ErbB therapeutic is part of a therapeutic regimen that comprises at least one additional treatment modality. In some embodiments, the at least one additional treatment modality is selected from the group consisting of: contacting said cell with one or more additional therapeutic agents, radiation, photodynamic therapy, laser therapy, and surgery.

In one aspect, the invention provides methods for identifying a subject as a candidate for treatment with an ErbB antagonist and a c-met antagonist, wherein said subject has been treated with an ErbB antagonist and suffers from cancer that has acquired resistance to said ErbB antagonist, comprising detecting a c-met activating mutation or c-met gene amplification in a cancer cell from said subject.

In one aspect, the invention provides methods for identifying a c-met antagonist comprising contacting a cancer cell that has acquired resistance to an ErbB antagonist, wherein said cancer cell comprises a c-met activating mutation or a c-met gene amplification, with an ErbB antagonist and a test compound and detecting a change in a cellular process selected from the group consisting of: decreased ErbB phosphorylation, decreased c-met phosphorylation, decreased ErbB-c-met association, decreased EGFR phosphorylation, decreased AKT phosphorylation, decreased cell growth, decreased cell proliferation and increased apoptosis, compared to said cellular process in an identical cell contacted only with an ErbB antagonist.

In one aspect, the invention provides methods for identifying a subject who is being treated with an ErbB antagonist and who is at risk for acquiring resistance to said ErbB antagonist, comprising detecting the presence of a c-met activating mutation or a c-met gene amplification in a cancer cell from said subject, wherein the presence of said c-met activating mutation or c-met gene amplification indicates a risk for acquiring said resistance.

In one aspect, the invention provides methods for producing a cell with acquired resistance to an ErbB antagonist comprising contacting a cell that is sensitive to an ErbB antagonist with at least one ErbB antagonist for at least 4 weeks and identifying cells that acquire resistance to said ErbB antagonist. In some embodiments, the cell does not comprise a mutation in an ErbB gene that confers resistance to said ErbB antagonist.

In one aspect, the invention provides cells produced by a method comprising a method for producing a cell with acquired resistance to an ErbB antagonist comprising contacting a cell that is sensitive to an ErbB antagonist with at least one ErbB antagonist for at least 4 weeks and identifying cells that acquire resistance to said ErbB antagonist.

In one aspect, the invention provides methods for treating a subject suffering from a cancer that is resistant to treatment with an ErbB antagonist, comprising administering to the subject an ErbB antagonist and an agent that inhibits HGF mediated activation of c-met.

In some embodiments, the agent is an antibody that prevents HGF from binding to c-met. In some embodiments, the antibody is an anti-HGF antibody. In some embodiments, the antibody is an anti-c-met antibody. In some embodiments, the ErbB is HER3. In some embodiments, the ErbB antagonist is an HER3 antagonist. In some embodiments, the cancer cell's growth and/or survival is promoted by ErbB.

In one aspect, the invention provides methods for treating a subject suffering from a cancer associated with an ErbB activating mutation or an ErbB gene amplification, wherein the subject has developed a resistance to treatment with an ErbB antagonist, comprising determining whether the subject has elevated c-met levels and/or activity, and administering to those subjects having elevated c-met activity an ErbB antagonist and a c-met antagonist.

In one aspect, the invention provides methods for treating a subject suffering from a cancer associated with an ErbB activating mutation or an ErbB gene amplification, comprising: (i) monitoring a subject being treated with an ErbB antagonist to determine if the subject develops elevated levels and/or c-met activity, and (ii) modifying the treatment regimen of the subject to include a c-met antagonist in addition to the ErbB antagonist where the subject has developed elevated c-met levels and/or activity.

In one aspect, the invention provides methods for treating a subject suffering from a cancer associated with an ErbB activating mutation or an ErbB gene amplification, comprising: (i) monitoring a subject being treated with ErbB antagonist to determine if the subject develops a resistance to the inhibitor, (ii) testing the subject to determine whether the subject has elevated c-met levels and/or activity, and (iii) modifying the treatment regimen of the subject to include a c-met antagonist in addition to the ErbB antagonist where the subject has elevated c-met levels and/or activity.

In some embodiments, the elevated c-met activity is associated with a c-met gene amplification, a c-met activating mutation, or HGF mediated c-met activation. In some embodiments, the HGF mediated c-met activation is associated with elevated HGF expression levels or elevated HGF activity. In some embodiments, the HGF mediated c-met activation is associated with an HGF gene amplification or an HGF activating mutation. In some embodiments, the c-met antagonist is an agent that inhibits HGF mediated activation of c-met. In some embodiments, the agent is an antibody that prevents HGF from binding to c-met. In some embodiments, the antibody is an anti-HGF antibody or an anti-c-met antibody.

In another aspect, the invention provides a method for reducing ErbB phosphorylation in a cancer cell by contacting the cell with an ErbB antagonist and a c-met antagonist.

In certain embodiments, the cancer cell has acquired a resistance to an ErbB antagonist and comprises elevated levels of c-met activity and/or expression, e.g., associated with, for example, an activating mutation in the c-met gene, a c-met gene amplification, or HGF mediated c-met activation. The methods disclosed herein may be used to reduce the phosphorylation of one or more of EGFR, HER2, HER3 and/or HER4.

In certain embodiments, it may be desirable to compare the level of ErbB phosphorylation in the cancer cell to a control, e.g., a cell that has not been contacted with an ErbB antagonist, a c-met antagonist, or both, or a cell that has been contacted with a different amount of one or both of the therapeutic agents, or a reference value, such as an expected value for a given assay, etc.

In another aspect, the invention provides a method for reducing PI3K mediated signaling in a cancer cell by contacting the cell with an ErbB antagonist and a c-met antagonist. In exemplary embodiments, the cancer cell has acquired a resistance to an ErbB antagonist and comprises elevated levels of c-met activity and/or expression (e.g., associated with an activating mutation in the c-met gene, a c-met gene amplification, or HGF mediated c-met activation). In certain embodiments, it may be desirable to compare the level of PDK mediated signaling in the cancer cell to a control, e.g., a cell that has not been contacted with an ErbB antagonist, a c-met antagonist or both, or a cell that has been contacted with a different amount of one or both of the therapeutic agents, or a reference value, such as an expected value for a given assay, etc.

In another aspect, the invention provides a method for reducing ErbB-mediated signaling in a cancer cell by contacting the cell with an ErbB antagonist and a c-met antagonist. In exemplary embodiments, the cancer cell has acquired a resistance to an ErbB antagonist and comprises elevated levels of c-met activity and/or expression, for example, associated with an activating mutation in the c-met gene, a c-met gene amplification, or HGF mediated c-met activation. The methods disclosed herein may be used to reduce signaling mediated by one or more of EGFR, HER2, HER3 and/or HER4. In certain embodiments, it may be desirable to compare the level of ErbB-mediated signaling in the cancer cell to a control, e.g., a cell that has not been contacted with an ErbB antagonist, a c-met antagonist, or both, or a cell that has been contacted with a different amount of one or both of the therapeutic agents, or a reference value, such as an expected value for a given assay, etc.

In another aspect, the invention provides a method for (i) restoring the sensitivity of a cancer cell to an ErbB antagonist, (ii) reducing resistance of a cancer cell to an ErbB antagonist, and/or (iii) treating acquired ErbB antagonist resistance in a cancer cell, by contacting the cell with an ErbB antagonist and a c-met antagonist. In exemplary embodiments, the cancer cell has acquired a resistance to an ErbB antagonist and comprises elevated levels of c-met activity and/or expression, e.g., associated with an activating mutation in the c-met gene, a c-met gene amplification, or HGF mediated c-met activation. The methods disclosed herein may be used to restore the sensitivity, reduce the resistance, and/or treat an acquired resistance, of a cancer cell to one or more of the following: an EGFR antagonist, an HER2 antagonist, an HER3 antagonist and/or an HER4 antagonist.

For example, an amount of cell growth and/or proliferation and/or amount of apoptosis may be determined in the presence of the ErbB antagonist/c-met antagonist combination therapy as compared to the ErbB antagonist alone. A decrease in the cell growth and/or proliferation and/or an increase in apoptosis of the cancer cell is indicative of an increase in sensitivity, or a reduction in resistance, to the ErbB antagonist.

In another aspect, the invention provides a method for reducing growth and/or proliferation of a cancer cell, or increasing apoptosis of a cancer cell, by contacting the cell with an ErbB antagonist and a c-met antagonist. In exemplary embodiments, the cancer cell has acquired a resistance to an ErbB antagonist and comprises elevated c-met activity and/or expression, e.g., associated with an activating mutation in the c-met gene, a c-met gene amplification, or HGF mediated c-met activation. In certain embodiments, it may be desirable to compare the level of growth and/or proliferation and/or apoptosis of the cancer cell to a control, e.g., a cell that has not been contacted with an ErbB antagonist, a c-met antagonist, or both, or a cell that has been contacted with a different amount of one or both of the therapeutic agents, or a reference value, such as an expected value for a given assay. The c-met antagonist can be administered serially or in combination with the EGFR antagonist, either in the same composition or as separate compositions. The administration of the c-met antagonist and the EGFR antagonist can be done simultaneously, e.g., as a single composition or as two or more distinct compositions, using the same or different administration routes. Alternatively, or additionally, the administration can be done sequentially, in any order. Alternatively, or additionally, the steps can be performed as a combination of both sequentially and simultaneously, in any order. In certain embodiments, intervals ranging from minutes to days, to weeks to months, can be present between the administrations of the two or more compositions. For example, the EGFR antagonist may be administered first, followed by the c-met antagonist. However, simultaneous administration or administration of the c-met antagonist first is also contemplated. Accordingly, in one aspect, the invention provides methods comprising administration of a c-met antagonist (such as an anti-c-met antibody), followed by administration of an EGFR antagonist (such as erlotinib (TARCEVA®)). In certain embodiments, intervals ranging from minutes to days, to weeks to months, can be present between the administrations of the two or more compositions.

In one aspect, the invention provides a composition for use in treating a cancer comprising an effective amount of a c-met antagonist and a pharmaceutically acceptable carrier, wherein said use comprises simultaneous or sequential administration of an EGFR antagonist. In some embodiments, the c-met antagonist is an anti-c-met antibody. In some embodiments, the EGFR antagonist is erlotinib (TARCEVA®).

In one aspect, the invention provides a composition for use in treating a cancer comprising an effective amount of a c-met antagonist and a pharmaceutically acceptable carrier, wherein said use comprises simultaneous or sequential administration of an EGFR antagonist. In some embodiments, the c-met antagonist is an anti-c-met antibody. In some embodiments, the EGFR antagonist is erlotinib (TARCEVA®).

Depending on the specific cancer indication to be treated, the combination therapy of the invention can be combined with additional therapeutic agents, such as chemotherapeutic agents, or additional therapies such as radiotherapy or surgery. Many known chemotherapeutic agents can be used in the combination therapy of the invention. Preferably those chemotherapeutic agents that are standard for the treatment of the specific indications will be used. Dosage or frequency of each therapeutic agent to be used in the combination is preferably the same as, or less than, the dosage or frequency of the corresponding agent when used without the other agent(s).

### BRIEF DESCRIPTION OF THE DRAWINGS

FIGS. 1A and 1B: Confirmation of EGFR and CMET mRNA coexpression in NSCLC cell lines and primary tumors by qRT-PCR. Expression of EGFR and MET mRNA was determined by quantitative RT-PCR in a panel of NSCLC cell lines (1A) or frozen primary NSCLC tumor lysates (1B). EGFR and CMET mRNA levels were positively correlated in cell lines (ρ=0.59, p<0.0001) and primary NSCLC specimens (ρ=0.48, p=0.0003).

FIG. 2: EBC1 shMet 4.12 cells (shMet 4.12) containing a tetracycline inducible shRNA directed against c-met or control shRNA directed against GFP (shGFP2) were grown in control media (Con) or media with 0.1 ug/ml tetracycline analog Doxycycline (Dox) for 48 hours. After serum-starvation for 2 hours, cells were untreated (−) or treated with TGFα (T, 20 nM) or Heregulin b1 (Hrg, 2 nM) for 20 minutes. Whole cell lysates were evaluated for expression of total and phospho-proteins as indicated. Beta-Actin (β-Actin) was detected to show equivalent loading between lanes.

FIG. 3: NSCLC H441 cells containing an inducible shRNA directed against c-met or control shRNA directed against GFP were grown in control media or media containing 0.1 ug/ml Dox (Dox) for 48 hours. After serum-starvation for 2 hours, cells were untreated (−) or treated with TGFα (T) or Heregulin b1 (H) for 20 minutes. Beta-Actin (β-Actin) (4th panel) was detected to show equivalent loading between lanes.

FIG. 4: Combination efficacy of erlotinib with shRNA knockdown of c-met in the EBC-1 NSCLC xenograft model. EBC-1-shMet-4.5 tumors were established in nude (CRL nu/nu) animals and then treated with either methylcellulose tween (MCT) vehicle plus drinking water containing 5% sucrose (Suc) (PO, QD where arrows indicate), MCT plus 1 mg/mL doxycycline (Dox) in the drinking water formulated in 5% sucrose (100 mg/kg; PO, QD, where arrows indicate), erlotinib plus drinking water containing 5% sucrose (PO, QD where arrows indicate), or erlotinib plus 1 mg/mL doxycycline in the drinking water formulated in 5% sucrose (PO, QD where arrows indicate). Oral dosing was done on days indicated by the arrows. Sucrose or Dox water was maintained throughout the study with bottles being interchanged every 2-3 days. Tumor volumes and SEM were calculated as described in the Examples.

FIG. 5: Combination efficacy of c-met antagonist MetMAb with EGFR antagonist erlotinib in the NCI-H596 hu-HGF-Tg-C3H-SCID xenograft model. NCI-H596 tumors were grown in hu-HGF-Tg-C3H-SCID or C3H-SCID littermate control animals and treated with either Captisol vehicle (PO, QD, ×2 weeks), erlotinib (150 mg/kg, PO, QD, ×2 weeks), MetMAb (30 mg/kg, IP, once), or the combination of MetMAb plus erlotinib at the same doses and schedules. Dosing was as indicated on the bottom of chart for MetMAb (open arrow head) and erlotinib or vehicle (closed arrow heads). Tumor measurements were taken by caliper twice to three times per week for about 9 weeks or until groups were removed from the study due to large tumor sizes within the group. Tumor volumes and SEM were calculated as described in the Examples.

FIG. 6: Time to tumor doubling (TTD) measurements, defined as the time it took for tumors to double in size, were calculated for each group and used to generate Kaplan-Meier survival curves. The combination of MetMAb plus erlotinib showed a dramatic improvement in tumor progression with a mean TTD of 49.5 (±2.6) days versus 17.8 (±2.2) days for the MetMAb-treated group, 9.5 (±1.2) days for the erlotinib-treated group, and 9.5 (±1.2) days for vehicle control group. The curves for the vehicle and erlotinib group were perfectly overlayed.

FIG. 7: depicts amino acid sequences of the framework regions (FR), hypervariable regions (HVR), first constant domain (CL or CH1) and Fc region (Fc) of one embodiment of an anti-c-met antibody. The Fc sequence depicted comprises mutations T366S, L368A and Y407V, as described in WO 2005/063816.

FIG. 8: depicts sequence of an Fc polypeptide comprising mutation T366W, as described in WO 2005/063816. In one embodiment, an Fc polypeptide comprising this sequence forms a complex with an Fc polypeptide comprising the Fc sequence of FIG. 7 to generate an Fc region.

FIGS. 9A-E: C-met activity regulates expression of EGFR ligands. A) Treatment with HGF induced upregulation of EGFR ligands in HGF-responsive NSCLC cell lines. Hop92 or NCI-H596 cells were serum-starved overnight, and then untreated (No HGF) or treated with HGF (50 ng/ml) for 6 hours (HGF). RNA from cells −/+ HGF treatment underwent microarray analyses as described in the Examples. RMA=relative microarray. B) C-met knock-down decreased expression of EGFR ligands ligand-independent NSCLC cell line EBC-1. Clones stably expressing shRNA directed against c-met (clones 3-15 and 4-12) were untreated (noDox) or treated with Doxycycline (Dox) for 24 or 48 hours. RNA from cells underwent microarray analyses as described in the Examples. C) EBC1shMet4-12 cell stably expressing shRNA directed against c-met were untreated (No Dox) or treated with Dox (Dox) for 24 hours without HGF (No HGF) or with HGF (100 ng/ml) for 2 hours. RNA from the cells underwent microarray analyses as described in the Examples. D) EBCshMet4-12 cells stably expressing shRNA directed against c-met and control cells stably expressing shGFP2 were untreated (No Dox) or treated with Dox (Dox) for 24 hours. RNA from the cells underwent microarray analyses as described in the Examples. E) Tumors from EBC1shMet-4.12 or EBCshMet-3.15 cells were established in nude (CRL nu-nu) animals, and mice were given drinking water with 1 mg/ml Dox (Dox) in 5% sucrose or 5% sucrose alone. After 3 days, TGFα levels in tumor lysates were evaluated by ELISA.

FIGS. 10A-C: (A) EBCshMet 4.12 or EBCshGFP2 cells were untreated (−) or treated with Dox (+) for 24, 48 or 72 hours. Protein lysates were evaluated for c-met, pEGFR or Her3 by western blotting. (B) EBCshMet 4.12 cells were treated with Dox (100 ng/ml) for 48 hours and analyzed by FACS for cell surface Her3. (C) Mice with EBCshMet 4.12 tumors were given drinking water with 1 mg/ml Dox in 5% sucrose (Dox) or 5% sucrose alone (Sucrose) for 3 days. Tumors lysates were evaluated for Her3 protein by western blotting.

FIG. 11: EBC-1 shMet cells (3.15 or 4.5 or 4.12) were untreated (−) or treated with 100 ng/ml Dox (+) for 96 hours alone or with HGF (5 or 100 ng/ml) or TGFa (1 or 50 nM) added 48 hours after initiation of Dox treatment. Cell number was evaluated using Cell Titer Glo.

FIG. 12: A time course experiment was performed with NCI-H596 cells in the presence (right panels) or absence (left panels) of HGF. Cell lysates were prepared at 10 minutes (10′), 24 hours (hr), 48 hours or 72 hours post-stimulation and western blots were performed to detect total c-met (top panel), phospho-EGFR (2nd panel), and total EGFR (3rd panel). Beta-Actin (β-Actin) (4th panel) was detected to show equivalent loading between lanes.

FIG. 13: NCI-H596 cells were plated in the presence of no ligands, TGF-α alone, TGF-α+HGF or HGF alone. Cell lysates were prepared at 10 minutes (10 min) and 24 hours (hr) post-stimulation, and immunoprecipitations (IP) for c-met were performed followed by western blotting for phospho-tyrosine (4G10; top panel), c-met (2nd panel), and EGFR (3rd panel). The phospho-tyrosine blots shows activation of EGFR (top band) and c-met (bottom band) in a ligand-dependent manner, attenuating after 24 hours. C-met immunoprecipitation brought down EGFR in all conditions regardless of activation status of EGFR or c-met.

FIG. 14: Viability assays were performed with NCI-H596 cells to evaluate response of cells to erlotinib in the presence of TGFα and varying concentrations of HGF as indicated. Reduction in relative response to erlotinib was detected as HGF levels increased from 0.5 ng/ml to 50 ng/ml.

FIG. 15: Viability assays were performed with NCI-H596 cells in the presence of TGFα and HGF (50 ng/ml), with or without MetMAb (1 μM), and varying concentrations of erlotinib. Data are represented as percent of untreated controls. Untreated control values are shown as individual points on top left of the figure.

FIG. 16: Combination treatment with MetMAb and Erlotinib resulted in more effective inhibition of phospho-Akt and phospho-ERK1/2. Human-HGF-transgenic-SCID (hu-HGF-Tg-SCID) mice bearing NCI-H596 tumors were treated with vehicles (MetMAb buffer (100 μL, IP) and methylcellulose tween (MCT, 100 μL, PO), MetMAb ((30 mg/kg, IP, once) and MCT), erlotinib ((100 mg/kg in MCT, 100 μL, PO) and MetMAb buffer (100 μL, IP)) or MetMAb and erlotinib (same dosing as described for each). MetMAb (or buffer) was dosed at time zero (t 0 hrs), erlotinib (or MCT) was dosed at time eighteen hours (t 18 hrs), mice were euthanized and tumors were collected at time twenty-four hours (t 24 hrs). Tumor lysates were analyzed for total and phospho-proteins by both direct Western blotting and immunoprecipitation followed by Western blot. Abbreviations: pTyr=phospho-tyrosine, EGFR=epidermal growth factor receptor, ERK (extracellular signal-regulated kinase-1 and 2. Beta-Actin (β-Actin) was detected to show equivalent loading between lanes.

FIGS. 17A and 17B: diagrammatically depict some of the results described in the present application.

### DETAILED DESCRIPTION

I. Definitions

The term “hepatocyte growth factor” or “HGF”, as used herein, refers, unless indicated otherwise, to any native or variant (whether native or synthetic) HGF polypeptide that is capable of activating the HGF/c-met signaling pathway under conditions that permit such process to occur. The term “wild type HGF” generally refers to a polypeptide comprising the amino acid sequence of a naturally occurring HGF protein. The term “wild type HGF sequence” generally refers to an amino acid sequence found in a naturally occurring HGF. C-met is a known receptor for HGF through which HGF intracellular signaling is biologically effectuated.

The term “HGF variant” as used herein refers to a HGF polypeptide which includes one or more amino acid mutations in the native HGF sequence. Optionally, the one or more amino acid mutations include amino acid substitution(s).

A “native sequence” polypeptide comprises a polypeptide having the same amino acid sequence as a polypeptide derived from nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

By “EGFR” (interchangeably termed “ErbB1”, “HER1” and “epidermal growth factor receptor”) is meant the receptor tyrosine kinase polypeptide Epidermal Growth Factor Receptor which is described in Ullrich et al, Nature (1984)309:418425, alternatively referred to as Her-1 and the c-erbB gene product, as well as variants thereof such as EGFRvIII. Variants of EGFR also include deletional, substitutional and insertional variants, for example those described in Lynch et al (New England Journal of Medicine 2004, 350:2129), Paez et al (Science 2004, 304:1497), Pao et al (PNAS 2004, 101:13306).

A “biological sample” (interchangeably termed “sample” or “tissue or cell sample”) encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. In some embodiments, the biological sample is obtained from a primary or metastatic tumor. The biological sample may contain compounds which are not naturally intermixed with the tissue in nature such as preservatives, anticoagulants, buffers, fixatives, nutrients, antibiotics, or the like.

A “c-met antagonist” (interchangeably termed “c-met inhibitor”) is an agent that interferes with c-met activation or function. Examples of c-met inhibitors include c-met antibodies; HGF antibodies; small molecule c-met antagonists; c-met tyrosine kinase inhibitors; antisense and inhibitory RNA (e.g., shRNA) molecules (see, for example, WO2004/87207). Preferably, the c-met inhibitor is an antibody or small molecule which binds to c-met. In a particular embodiment, a c-met inhibitor has a binding affinity (dissociation constant) to c-met of about 1,000 nM or less. In another embodiment, a c-met inhibitor has a binding affinity to c-met of about 100 nM or less. In another embodiment, a c-met inhibitor has a binding affinity to c-met of about 50 nM or less. In a particular embodiment, a c-met inhibitor is covalently bound to c-met. In a particular embodiment, a c-met inhibitor inhibits c-met signaling with an IC50 of 1,000 nM or less. In another embodiment, a c-met inhibitor inhibits c-met signaling with an IC50 of 500 nM or less. In another embodiment, a c-met inhibitor inhibits c-met signaling with an IC50 of 50 nM or less.

As used herein, the term “c-met-targeted drug” refers to a therapeutic agent that binds to c-met and inhibits c-met activation. An example of a c-met targeted drug is MetMAb (OA5D5.v2).

“C-met activation” refers to activation, or phosphorylation, of the c-met receptor. Generally, c-met activation results in signal transduction (e.g. that caused by an intracellular kinase domain of a c-met receptor phosphorylating tyrosine residues in c-met or a substrate polypeptide). C-met activation may be mediated by c-met ligand (HGF) binding to a c-met receptor of interest. HGF binding to c-met may activate a kinase domain of c-met and thereby result in phosphorylation of tyrosine residues in the c-met and/or phosphorylation of tyrosine residues in additional substrate polypeptides(s).

An “EGFR antagonist” (interchangeably termed “EGFR inhibitor”) is an agent that interferes with EGFR activation or function. Examples of EGFR inhibitors include EGFR antibodies; EGFR ligand antibodies; small molecule EGFR antagonists; EGFR tyrosine kinase inhibitors; antisense and inhibitory RNA (e.g., shRNA) molecules (see, for example, WO2004/87207). Preferably, the EGFR inhibitor is an antibody or small molecule which binds to EGFR. In some embodiments, the EGFR inhibitor is an EGFR-targeted drug. In a particular embodiment, an EGFR inhibitor has a binding affinity (dissociation constant) to EGFR of about 1,000 nM or less. In another embodiment, an EGFR inhibitor has a binding affinity to EGFR of about 100 nM or less. In another embodiment, an EGFR inhibitor has a binding affinity to EGFR of about 50 nM or less. In a particular embodiment, an EGFR inhibitor is covalently bound to EGFR. In a particular embodiment, an EGFR inhibitor inhibits EGFR signaling with an IC50 of 1,000 nM or less. In another embodiment, an EGFR inhibitor inhibits EGFR signaling with an IC50 of 500 nM or less. In another embodiment, an EGFR inhibitor inhibits EGFR signaling with an IC50 of 50 nM or less.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{new}. Preferred HER2 is native sequence human HER2.

“ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989).

The terms “ErbB4” and “HER4” herein refer to the receptor polypeptide as disclosed, for example, in EP Pat Appln No 599,274; Plowman et al., Proc. Natl. Acad Sci. USA, 90:1746-1750 (1993); and Plowman et al., Nature, 366:473-475 (1993), including isoforms thereof, e.g., as disclosed in WO99/19488, published Apr. 22, 1999.

As used herein, “ErbB” refers to the receptor polypeptides EGFR, HER2, HER3, and HER4.

“EGFR activation” refers to activation, or phosphorylation, of EGFR. Generally, EGFR activation results in signal transduction (e.g. that caused by an intracellular kinase domain of EGFR receptor phosphorylating tyrosine residues in EGFR or a substrate polypeptide). EGFR activation may be mediated by EGFR ligand binding to a EGFR dimer comprising EGFR. EGFR ligand binding to a EGFR dimer may activate a kinase domain of one or more of the EGFR in the dimer and thereby results in phosphorylation of tyrosine residues in one or more of the EGFR and/or phosphorylation of tyrosine residues in additional substrate polypeptides(s).

“c-met activation” refers to activation, or phosphorylation, of c-met. Generally, c-met activation results in signal transduction (e.g. that caused by an intracellular kinase domain of c-met receptor phosphorylating tyrosine residues in c-met or a substrate polypeptide). C-met activation may be mediated by c-met ligand (e.g., HGF) binding to a c-met dimer. C-met ligand binding to a c-met dimer may activate a kinase domain of one or more of the c-met in the dimer and thereby results in phosphorylation of tyrosine residues in one or more of the c-met and/or phosphorylation of tyrosine residues in additional substrate polypeptides(s).

As used herein, the term “EGFR-targeted drug” refers to a therapeutic agent that binds to EGFR and inhibits EGFR activation. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943, 533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small molecules that bind to EGFR include ZD1839 or Gefitinib (IRESSA; Astra Zeneca); CP-358774 or Erlotinib (TARCEVA™; Genentech/OSI); and AG1478, AG1571 (SU 5271; Sugen); EMD-7200.

By “EGFR resistant” cancer is meant that the cancer patient has progressed while receiving an EGFR antagonist therapy (i.e., the patient is “EGFR refractory”), or the patient has progressed within 12 months (for instance, within one, two, three, or six months) after completing an EGFR antagonist-based therapy regimen. For example, cancers which incorporate T790M mutant EGFR are resistant to erlotinib and gefitinib therapy.

By “erlotinib or gefitinib resistant” cancer is meant that the cancer patient has progressed while receiving erlotinib- or gefitinib-based therapy (i.e., the patient is “erlotinib or gefitinib refractory”), or the patient has progressed within 12 months (for instance, within one, two, three, or six months) after completing an erlotinib- or gefitinib-based therapy regimen.

The term “ligand-independent” as used herein, as for example applied to receptor signaling activity, refers to signaling activity that is not dependent on the presence of a ligand. For example, EGFR signaling may result from dimerization with other members of the HER family such as HER2. A receptor having ligand-independent kinase activity will not necessarily preclude the binding of ligand to that receptor to produce additional activation of the kinase activity.

The term “constitutive” as used herein, as for example applied to receptor kinase activity, refers to continuous signaling activity of a receptor that is not dependent on the presence of a ligand or other activating molecules. For example, EGFR variant III (EGFRvIII) which is commonly foundin glioblastoma multiforme has deleted much of its extracellular domain. Although ligands are unable to bind EGFRvIII it is nevertheless continuously active and is associated with abnormal proliferation and survival. Depending on the nature of the receptor, all of the activity may be constitutive or the activity of the receptor may be further activated by the binding of other molecules (e. g. ligands). Cellular events that lead to activation of receptors are well known among those of ordinary skill in the art. For example, activation may include oligomerization, e.g., dimerization, trimerization, etc., into higher order receptor complexes. Complexes may comprise a single species of protein, i.e., a homomeric complex. Alternatively, complexes may comprise at least two different protein species, i.e., a heteromeric complex. Complex formation may be caused by, for example, overexpression of normal or mutant forms of receptor on the surface of a cell. Complex formation may also be caused by a specific mutation or mutations in a receptor.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

A “tyrosine kinase inhibitor” is a molecule which inhibits to some extent tyrosine kinase activity of a tyrosine kinase such as a c-met receptor.

A cancer or biological sample which “displays c-met and/or EGFR expression, amplification, or activation” is one which, in a diagnostic test, expresses (including overexpresses) c-met and/or EGFR, has amplified c-met and/or EGFR gene, and/or otherwise demonstrates activation or phosphorylation of a c-met and/or EGFR.

A cancer or biological sample which “does not display c-met and/or EGFR expression, amplification, or activation” is one which, in a diagnostic test, does not express (including overexpress) c-met and/or EGFR, does not have amplified c-met and/or EGFR gene, and/or otherwise does not demonstrate activation or phosphorylation of a c-met and/or EGFR.

A cancer or biological sample which “displays c-met and/or EGFR activation” is one which, in a diagnostic test, demonstrates activation or phosphorylation of c-met and/or EGFR. Such activation can be determined directly (e.g. by measuring c-met and/or EGFR phosphorylation by ELISA) or indirectly.

A cancer or biological sample which “does not display c-met and/or EGFR activation” is one which, in a diagnostic test, does not demonstrate activation or phosphorylation of a c-met and/or EGFR. Such activation can be determined directly (e.g. by measuring c-met and/or EGFR phosphorylation by ELISA) or indirectly.

A cancer or biological sample which “displays constitutive c-met and/or EGFR activation” is one which, in a diagnostic test, demonstrates constitutive activation or phosphorylation of a c-met and/or EGFR. Such activation can be determined directly (e.g. by measuring c-met and/or EGFR phosphorylation by ELISA) or indirectly.

A cancer or biological sample which “does not display c-met and/or EGFR amplification” is one which, in a diagnostic test, does not have amplified c-met and/or EGFR gene.

A cancer or biological sample which “displays c-met and/or EGFR amplification” is one which, in a diagnostic test, has amplified c-met and/or EGFR gene.

A cancer or biological sample which “does not display constitutive c-met and/or EGFR activation” is one which, in a diagnostic test, does not demonstrate constitutive activation or phosphorylation of a c-met and/or EGFR. Such activation can be determined directly (e.g. by measuring c-met and/or EGFR phosphorylation by ELISA) or indirectly.

A cancer or biological sample which “displays ligand-independent c-met and/or EGFR activation” is one which, in a diagnostic test, demonstrates ligand-independent activation or phosphorylation of a c-met and/or EGFR. Such activation can be determined directly (e.g. by measuring c-met and/or EGFR phosphorylation by ELISA) or indirectly.

A cancer or biological sample which “does not display ligand-independent c-met and/or EGFR activation” is one which, in a diagnostic test, demonstrates ligand-independent activation or phosphorylation of a c-met and/or EGFR. Such activation can be determined directly (e.g. by measuring c-met and/or EGFR phosphorylation by ELISA) or indirectly.

“Phosphorylation” refers to the addition of one or more phosphate group(s) to a protein, such as a EGFR and/or c-met, or substrate thereof.

A “phospho-ELISA assay” herein is an assay in which phosphorylation of one or more c-met and/or EGFR is evaluated in an enzyme-linked immunosorbent assay (ELISA) using a reagent, usually an antibody, to detect phosphorylated c-met and/or EGFR, substrate, or downstream signaling molecule. Preferably, an antibody which detects phosphorylated c-met and/or EGFR is used. The assay may be performed on cell lysates, preferably from fresh or frozen biological samples.

A cancer cell with “c-met and/or EGFR overexpression or amplification” is one which has significantly higher levels of a c-met and/or EGFR protein or gene compared to a noncancerous cell of the same tissue type. Such overexpression may be caused by gene amplification or by increased transcription or translation. c-met and/or EGFR overexpression or amplification may be determined in a diagnostic or prognostic assay by evaluating increased levels of the c-met and/or EGFR protein present on the surface of a cell (e.g. via an immunohistochemistry assay; IHC). Alternatively, or additionally, one may measure levels of c-met and/or EGFR-encoding nucleic acid in the cell, e.g. via fluorescent in situ hybridization (FISH; see WO98/45479 published October, 1998), southern blotting, or polymerase chain reaction (PCR) techniques, such as quantitative real time PCR (qRT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A cancer cell which “does not overexpress or amplify c-met and/or EGFR” is one which does not have higher than normal levels of c-met and/or EGFR protein or gene compared to a noncancerous cell of the same tissue type.

The term “mutation”, as used herein, means a difference in the amino acid or nucleic acid sequence of a particular protein or nucleic acid (gene, RNA) relative to the wild-type protein or nucleic acid, respectively. A mutated protein or nucleic acid can be expressed from or found on one allele (heterozygous) or both alleles (homozygous) of a gene, and may be somatic or germ line. In the instant invention, mutations are generally somatic. Mutations include sequence rearrangements such as insertions, deletions, and point mutations (including single nucleotide/amino acid polymorphisms).

To “inhibit” is to decrease or reduce an activity, function, and/or amount as compared to a reference.

Protein “expression” refers to conversion of the information encoded in a gene into messenger RNA (mRNA) and then to the protein.

Herein, a sample or cell that “expresses” a protein of interest (such as a HER receptor or HER ligand) is one in which mRNA encoding the protein, or the protein, including fragments thereof, is determined to be present in the sample or cell.

An “immunoconjugate” (interchangeably referred to as “antibody-drug conjugate,” or “ADC”) means an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. The Fc sequence of an immunoglobulin generally comprises two constant domains, a CH2 domain and a CH3 domain, and optionally comprises a CH4 domain. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

By “Fc polypeptide” herein is meant one of the polypeptides that make up an Fc region. An Fc polypeptide may be obtained from any suitable immunoglobulin, such as IgG₁, IgG₂, IgG₃, or IgG₄ subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “antibody” is used in the broadest sense and specifically covers monoclonal antibodies (including full length monoclonal antibodies), polyclonal antibodies, multispecific antibodies (e.g., bispecific antibodies), monovalent antibodies, multivalent antibodies, and antibody fragments so long as they exhibit the desired biological activity.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment. In one embodiment, an antibody of the invention is a one-armed antibody as described in WO2005/063816. In one embodiment, the one-armed antibody comprises Fc mutations constituting “knobs” and “holes” as described in WO2005/063816. For example, a hole mutation can be one or more of T366A, L368A and/or Y407V in an Fc polypeptide, and a cavity mutation can be T366W.

A “blocking” antibody or an antibody “antagonist” is one which inhibits or reduces biological activity of the antigen it binds. Preferred blocking antibodies or antagonist antibodies completely inhibit the biological activity of the antigen.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface or the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

A “naked antibody” is an antibody that is not conjugated to a heterologous molecule, such as cytotoxic moiety or radiolabel.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

To increase the half-life of the antibodies or polypeptide containing the amino acid sequences of this invention, one can attach a salvage receptor binding epitope to the antibody (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule (e.g., Ghetie et al., Ann. Rev. Immunol. 18:739-766 (2000), Table 1). Antibodies with substitutions in an Fc region thereof and increased serum half-lives are also described in WO00/42072, WO 02/060919; Shields et al., J. Biol. Chem. 276:6591-6604 (2001); Hinton, J. Biol. Chem. 279:6213-6216 (2004)). In another embodiment, the serum half-life can also be increased, for example, by attaching other polypeptide sequences. For example, antibodies or other polypeptides useful in the methods of the invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In one preferred embodiment, the serum albumin peptide to be attached comprises an amino acid sequence of DICLPRWGCLW (SEQ ID NO:21). In another embodiment, the half-life of a Fab is increased by these methods. See also, Dennis et al. J. Biol. Chem. 277:35035-35043 (2002) for serum albumin binding peptide sequences.

An “isolated” polypeptide or “isolated” antibody is one that has been identified and separated and/or recovered from a component of its natural environment. Contaminant components of its natural environment are materials that would interfere with diagnostic or therapeutic uses for the polypeptide or antibody, and may include enzymes, hormones, and other proteinaceous or nonproteinaceous solutes. In preferred embodiments, the polypeptide or antibody will be purified (1) to greater than 95% by weight of polypeptide or antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide or antibody includes the polypeptide or antibody in situ within recombinant cells since at least one component of the polypeptide's natural environment will not be present. Ordinarily, however, isolated polypeptide or antibody will be prepared by at least one purification step.

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain 10, 20, 30, 40, 50, 60, 70, 80, 90, or 100, 200, 300, 400, 500, 600, or more nucleotides or 10, 20, 30, 40, 50, 60, 70, 80, 90, 100, 120, 140, 160, 180, 190, 200 amino acids or more.

“Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer, colon cancer, rectal cancer, colorectal cancer, endometrial or uterine carcinoma, salivary gland carcinoma, kidney or renal cancer (such as renal cell carcinoma), prostate cancer, vulva(cancer, thyroid cancer, hepatic carcinoma, anal carcinoma, penile carcinoma, testicular cancer, esophagael cancer, tumors of the biliary tract, as well as head and neck cancer.

The term “pre-cancerous” refers to a condition or a growth that typically precedes or develops into a cancer. A “pre-cancerous” growth will have cells that are characterized by abnormal cell cycle regulation, proliferation, or differentiation, which can be determined by markers of cell cycle regulation, cellular proliferation, or differentiation.

By “dysplasia” is meant any abnormal growth or development of tissue, organ, or cells. Preferably, the dysplasia is high grade or precancerous.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass.

Both stimulatory and inhibitory molecular pathways within the tumor cell regulate this behavior, and interactions between the tumor cell and host cells in the distant site are also significant.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

By “primary tumor” or “primary cancer” is meant the original cancer and not a metastatic lesion located in another tissue, organ, or location in the subject's body.

By “benign tumor” or “benign cancer” is meant a tumor that remains localized at the site of origin and does not have the capacity to infiltrate, invade, or metastasize to a distant site.

By “tumor burden” is meant the number of cancer cells, the size of a tumor, or the amount of cancer in the body. Tumor burden is also referred to as tumor load.

By “tumor number” is meant the number of tumors.

By “subject” is meant a mammal, including, but not limited to, a human or non-human mammal, such as a bovine, equine, canine, ovine, or feline. Preferably, the subject is a human.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma II and calicheamicin omega II (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

### Therapeutic Agents

The present invention features the use of c-met antagonists and EGFR antagonists in combination therapy to treat a pathological condition, such as cancer, in a subject.

### C-met Antagonists

C-met antagonists useful in the methods of the invention include polypeptides that specifically bind to c-met, anti-c-met antibodies, c-met small molecules, receptor molecules and derivatives which bind specifically to c-met, and fusions proteins. C-met antagonists also include antagonistic variants of c-met polypeptides, RNA aptamers and peptibodies against c-met and HGF. Also included as c-met antagonists useful in the methods of the invention are anti-HGF antibodies, anti-HGF polypeptides, c-met receptor molecules and derivatives which bind specifically to HGF. Examples of each of these are described below,

Anti-c-met antibodies that are useful in the methods of the invention include any antibody that binds with sufficient affinity and specificity to c-met and can reduce or inhibit c-met activity. The antibody selected will normally have a sufficiently strong binding affinity for c-met, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. Preferably, the anti-c-met antibody of the invention can be used as a therapeutic agent in targeting and interfering with diseases or conditions wherein c-met/HGF activity is involved. Also, the antibody may be subjected to other biological activity assays, e.g., in order to evaluate its effectiveness as a therapeutic. Such assays are known in the art and depend on the target antigen and intended use for the antibody.

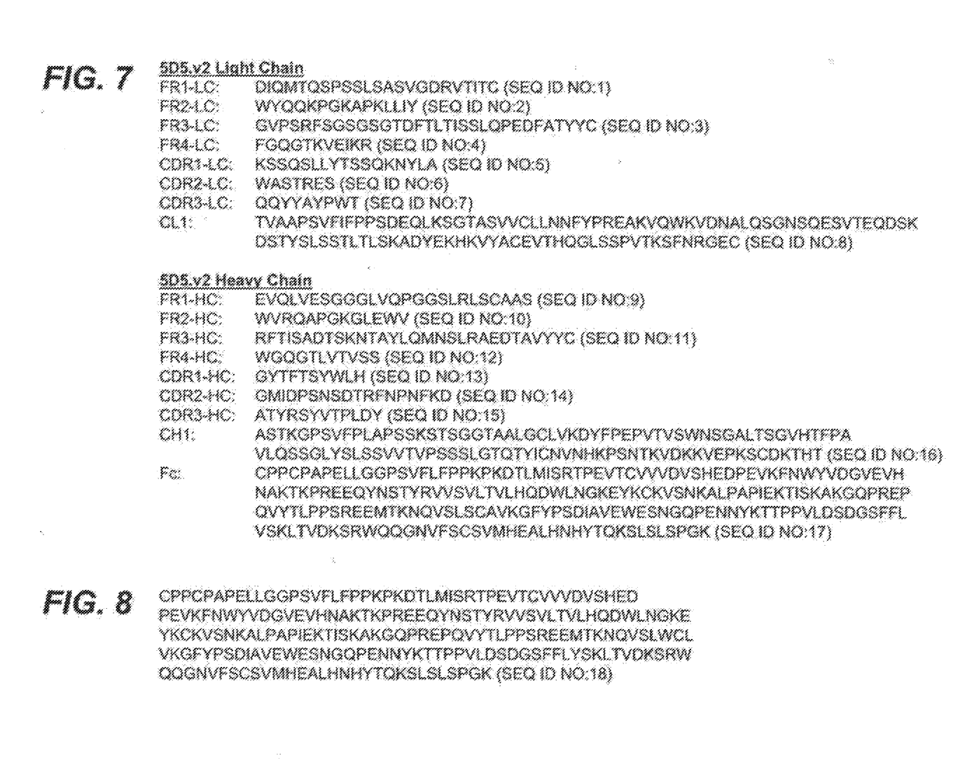

Anti-c-met antibodies are known in the art (see, e.g., Martens, T, et al (2006) Clin Cancer Res 12(20 Pt 1):6144; U.S. Pat. No. 6,468,529; WO2006/015371; WO2007/063816; U.S. Pat. No. 7,408,043; WO2009/007427; WO2005/016382; WO2007/126799. In one embodiment, the anti-c-met antibody comprises a heavy chain variable domain comprising one or more of CDR1-HC, CDR2-HC and CDR3-HC sequence depicted in FIG. 7 (SEQ ID NO: 13-15). In some embodiments, the antibody comprises a light chain variable domain comprising one or more of CDR1-LC, CDR2-LC and CDR3-LC sequence depicted in FIG. 7 (SEQ ID NO: 5-7). In some embodiments, the heavy chain variable domain comprises FR1-HC, FR2-HC, FR3-HC and FR4-HC sequence depicted in FIG. 7 (SEQ ID NO: 9-12). In some embodiments, the light chain variable domain comprises FR1-LC, FR2-LC, FR3-LC and FR4-LC sequence depicted in FIG. 7 (SEQ ID NO: 1-4). In some embodiments, the anti-c-met antibody is monovalent and comprises an Fc region. In some embodiments, the antibody comprises Fc sequence depicted in FIG. 7 (SEQ ID NO: 17).

In some embodiments, the antibody is monovalent and comprises a Fc region, wherein the Fc region comprises a first and a second polypeptide, wherein the first polypeptide comprises the Fc sequence depicted in FIG. 7 (SEQ ID NO: 17) and the second polypeptide comprises the Fc sequence depicted in FIG. 8 (SEQ ID NO: 18).

In one embodiment, the anti-c-met antibody comprises (a) a first polypeptide comprising a heavy chain variable domain having the sequence: QVQLQQSGPELVRPGASVKMSCRASGYTFTSYWLHWVKQRPGQGLEWIGMIDPSNSDTRFN PNFKDKATLNVDRSSNTAYMLLSSLTSADSAVYYCATYGSYVSPLDYWGQGTSVTVSS (SEQ ID NO:19), CH1 sequence depicted in FIG. 7 (SEQ ID NO: 16), and the Fc sequence depicted in FIG. 7 (SEQ ID NO: 17); and (b) a second polypeptide comprising a light chain variable domain having the sequence: DIMMSQSPSSLTVSVGEKVTVSCKSSQSLLYTSSQKNYLAWYQQKPGQSPKLLIYWASTRES GVPDRFTGSGSGTDFTLTITSVKADDLAVYYCQQYYAYPWTFGGGTKLEIK (SEQ NO:20), and CL1 sequence depicted in FIG. 7 (SEQ ID NO: 8); and (c) a third polypeptide comprising the Fc sequence depicted in FIG. 8 (SEQ ID NO: 18).

In other embodiments, the anti-c-met antibody is the monoclonal antibody produced by the hybridoma cell line deposited under American Type Culture Collection Accession Number ATCC HB-11894 (hybridoma 1A3.3.13) or HB-11895 (hybridoma 5D5.11.6). In other embodiments, the antibody comprises one or more of the CDR sequences of the monoclonal antibody produced by the hybridoma cell line deposited under American Type Culture Collection Accession Number ATCC HB-11894 (hybridoma 1A3.3.13) or HB-11895 (hybridoma 5D5.11.6).

In other embodiments, a c-met antibody of the invention specifically binds at least a portion of c-met Sema domain or variant thereof. In one example, an antagonist antibody of the invention specifically binds at least one of the sequences selected from the group consisting of LDAQT (SEQ ID NO: 38) (e.g., residues 269-273 of c-met), LTEKRKKRS (SEQ ID NO: 39) (e.g., residues 300-308 of c-met), KPDSAEPM (SEQ ID NO: 40) (e.g., residues 350-357 of c-met) and NVRCLQHF (SEQ ID NO: 41) (e.g., residues 381-388 of c-met). In one embodiment, an antagonist antibody of the invention specifically binds a conformational epitope formed by part or all of at least one of the sequences selected from the group consisting of LDAQT (SEQ ID NO: 38) (e.g., residues 269-273 of c-met), LTEKRKKRS (SEQ ID NO: 39) (e.g., residues 300-308 of c-met), KPDSAEPM (SEQ ID NO: 40) (e.g., residues 350-357 of c-met) and NVRCLQHF (SEQ ID NO: 41) (e.g., residues 381-388 of c-met). In one embodiment, an antagonist antibody of the invention specifically binds an amino acid sequence having at least 50%, 60%, 70%, 80%, 90%, 95%, 98% sequence identity or similarity with the sequence LDAQT (SEQ ID NO: 38), LTEKRKKRS (SEQ ID NO: 39), KPDSAEPM (SEQ ID NO: 40) and/or NVRCLQHF (SEQ ID NO: 41).

Anti-HGF antibodies are well known in the art. See, e.g., Kim K J, et al. Clin Cancer Res. (2006) 12(4):1292-8; WO2007/115049; WO2009/002521; WO2007/143098; WO2007/017107; WO2005/017107; L2G7; AMG-102.

C-met receptor molecules or fragments thereof that specifically bind to HGF can be used in the methods of the invention, e.g., to bind to and sequester the HGF protein, thereby preventing it from signaling. Preferably, the c-met receptor molecule, or HGF binding fragment thereof, is a soluble form. In some embodiments, a soluble form of the receptor exerts an inhibitory effect on the biological activity of the c-met protein by binding to HGF, thereby preventing it from binding to its natural receptors present on the surface of target cells. Also included are c-met receptor fusion proteins, examples of which are described below.

A soluble c-met receptor protein or chimeric c-met receptor proteins of the present invention includes c-met receptor proteins which are not fixed to the surface of cells via a transmembrane domain. As such, soluble forms of the c-met receptor, including chimeric receptor proteins, while capable of binding to and inactivating HGF, do not comprise a transmembrane domain and thus generally do not become associated with the cell membrane of cells in which the molecule is expressed. See, e.g., Kong-Beltran, M et al Cancer Cell (2004) 6(1): 75-84.

HGF molecules or fragments thereof that specifically bind to c-met and block or reduce activation of c-met, thereby preventing it from signaling, can be used in the methods of the invention.

Aptamers are nucleic acid molecules that form tertiary structures that specifically bind to a target molecule, such as a HGF polypeptide. The generation and therapeutic use of aptamers are well established in the art. See, e.g., U.S. Pat. No, 5,475,096. A HGF aptamer is a pegylated modified oligonucleotide, which adopts a three-dimensional conformation that enables it to bind to extracellular HGF. Additional information on aptamers can be found in U.S. Patent Application Publication No. 20060148748.

A peptibody is a peptide sequence linked to an amino acid sequence encoding a fragment or portion of an immunoglobulin molecule. Polypeptides may be derived from randomized sequences selected by any method for specific binding, including but not limited to, phage display technology. In a preferred embodiment, the selected polypeptide may be linked to an amino acid sequence encoding the Fc portion of an immunoglobulin. Peptibodies that specifically bind to and antagonize HGF or c-met are also useful in the methods of the invention.

C-met antagonists include small molecules such as compounds described in U.S. Pat. No. 5,792,783; U.S. Pat. No. 5,834,504; U.S. Pat. No. 5,880,141; U.S. Pat. No. 6,297,238; U.S. Pat. No. 6,599,902; U.S. Pat. No. 6,790,852; US 2003/0125370; US 2004/0242603; US 2004/0198750; US 2004/0110758; US 2005/0009845; US 2005/0009840; US 2005/0245547; US 2005/0148574; US 2005/0101650; US 2005/0075340; US 2006/0009453; US 2006/0009493; WO 98/007695; WO 2003/000660; WO 2003/087026; WO 2003/097641; WO 2004/076412; WO 2005/004808; WO 2005/121 125; WO 2005/030140; WO 2005/070891; WO 2005/080393; WO 2006/014325; WO 2006/021886; WO 2006/021881, WO 2007/103308). PHA-665752 is a small molecule, ATP-competitive, active-site inhibitor of the catalytic activity of c-Met, as well as cell growth, cell motility, invasion, and morphology of a variety of tumor cells (Ma et al (2005) Clin. Cancer Res. 11:2312-2319; Christensen et al (2003) Cancer Res. 63:7345-7355).

### EGFR Antagonists

EGFR antagonists include antibodies such as humanized monoclonal antibody known as nimotuzumab (YM Biosciences), fully human ABX-EGF (panitumumab, Abgenix Inc.) as well as fully human antibodies known as E1.1, E2.4, E2.5, E6.2, E6.4, E2.11, E6.3 and E7.6.3 and described in U.S. Pat. No. 6,235,883; MDX-447 (Medarex Inc). Pertuzumab (2C4) is a humanized antibody that binds directly to HER2 but interferes with HER2-EGFR dimerization thereby inhibiting EGFR signaling. Other examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH).

Anti-EGFR antibodies that are useful in the methods of the invention include any antibody that binds with sufficient affinity and specificity to EGFR and can reduce or inhibit EGFR activity. The antibody selected will normally have a sufficiently strong binding affinity for EGFR, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. Preferably, the anti-c-met antibody of the invention can be used as a therapeutic agent in targeting and interfering with diseases or conditions wherein EGFR/EGFR ligand activity is involved. Also, the antibody may be subjected to other biological activity assays, e.g., in order to evaluate its effectiveness as a therapeutic. Such assays are known in the art and depend on the target antigen and intended use for the antibody.

Bispecific antibodies are antibodies that have binding specificities for at least two different epitopes. Exemplary bispecific antibodies may bind to EGFR and to c-met. In another example, an exemplary bispecific antibody may bind to two different epitopes of the same protein, e.g., c-met protein. Alternatively, a c-met or EGFR arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the c-met or EGFR-expressing cell. Bispecific antibodies may also be used to localize cytotoxic agents to cells which express EGFR or c-met. These antibodies possess a EGFR or c-met-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

EGFR antagonists also include small molecules such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, WO9935146, WO0132651, U.S. Pat. No. 6,344,455, U.S. Pat. No. 5,760,041, U.S. Pat. No. 6,002,008, U.S. Pat. No. 5,747,498. Particular small molecule EGFR antagonists include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa® (ZD1839, gefitinib, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-1H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide); lapatinib (Tykerb, GlaxoSmithKline); ZD6474 (Zactima, AstraZeneca); CUDC-101 (Curis); canertinib (CI-1033); AEE788 (6-[4-[(4-ethyl-1-piperazinyl)methyl]phenyl]-N-[(1R)-1-phenylethyl]-7H-pyrrolo[2,3-d]pyrimidin-4-amine, WO2003013541, Novartis) and PKI166 4-[4-[[(1R)-1-phenylethyl]amino]-7H-pyrrolo[2,3-d]pyrimidin-6-yl]-phenol, WO9702266 Novartis).

In a particular embodiment, the EGFR antagonist has a general formula I:

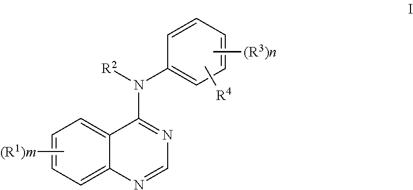

in accordance with U.S. Pat. No. 5,757,498, incorporated herein by reference, wherein:

m is 1, 2, or 3;

each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;

or each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶-piperazin-1-yl, imidazol-1-yl, 4-pyridon-1-yl, —(C₁-C₄ alkylene)(CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵,—OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;

or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein said —NHSO²R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;

or two R¹ groups are taken together with the carbons to which they are attached to form a 5-8 membered ring that includes 1 or 2 heteroatoms selected from O, S and N;

R² is hydrogen or C₁-C₆ alkyl optionally substituted by 1 to 3 substituents independently selected from halo, C₁-C₄ alkoxy, —NR⁶R⁶, and —SO₂R⁵;

n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and

R⁴ is azido or -(ethynyl)-R¹¹ wherein R¹¹ is hydrogen or C₁-C₆ alkyl optionally substituted by hydroxy, —OR⁶, or —NR⁶R⁶.

In a particular embodiment, the EGFR antagonist is a compound according to formula I selected from the group consisting of:

(6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine, (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-}6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin-4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl]-(5-ethynyl-2-methyl-phenyl)-amine; [6,7-bis-(2-methoxy-ethoxy)-quinazolin-4-yl]-(3-ethynyl-4-fluoro-phenyl)-amine; [6,7-bis-(2-chloro-ethoxy)-quinazolin-4-yl]-(3-ethynyl-phenyl)-amine; [6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7-yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-[2-(4-methyl-piperazin-1-yl)-ethoxy}-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; 2-[4-(3-ethynyl-phenylamino)-6-(2-methoxy-ethoxy)-quinazolin-7-yloxy]-ethanol; (6,7-dipropoxy-quinazolin-4-yl)-(3-ethynyl-phenyl)-amine; (6,7-diethoxy-quinazolin-4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine, (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; and (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine.

In a particular embodiment, the EGFR antagonist of formula I is N-(3-ethynylphenyl)-6,7-bis(2-methoxyethoxy)-4-quinazolinamine. In a particular embodiment, the EGFR antagonist N-(3-ethynylphenyl)-6,7-bis(2-methoxyethoxy)-4-quinazolinamine is in HCl salt form. In another particular embodiment, the EGFR antagonist N-(3-ethynylphenyl)-6,7-bis(2-methoxyethoxy)-4-quinazolinamine is in a substantially homogeneous crystalline polymorph form (described as polymorph B in WO 01/34,574) that exhibits an X-ray powder diffraction pattern having characteristic peaks expressed in degrees 2-theta at approximately 6.26, 12.48, 13.39, 16.96, 20.20, 21.10, 22.98, 24.46, 25.14 and 26.91. Such polymorph form of N-(3-ethynylphenyl)-6,7-bis(2-methoxyethoxy)-4-quinazolinamine is referred to as Tarceva™ as well as OSI-774, CP-358774 and erlotinib.

The compounds of formula I, pharmaceutically acceptable salts and prodrugs thereof (hereafter the active compounds) may be prepared by any process known to be applicable to the preparation of chemically-related compounds. In general the active compounds may be made from the appropriately substituted quinazoline using the appropriately substituted amine as shown in the general scheme I disclosed in U.S. Pat. No. 5,747,498:

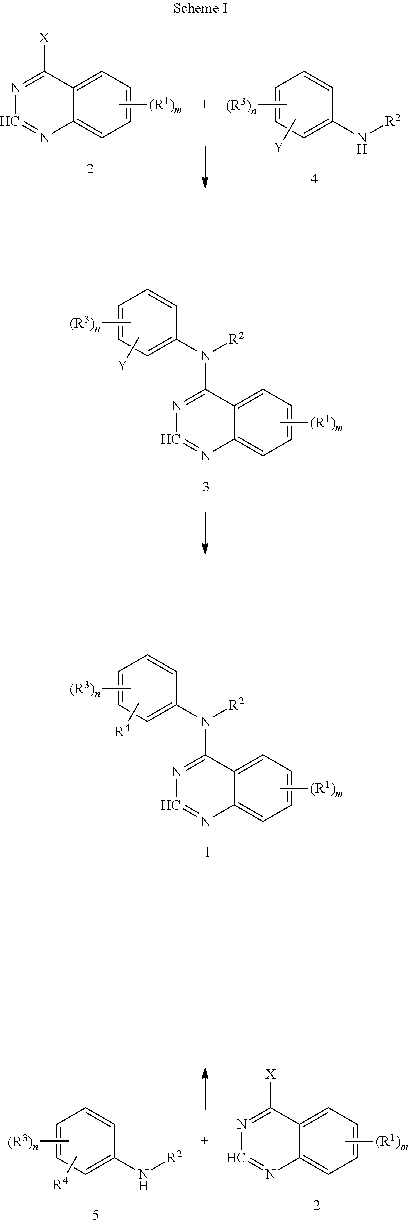

As shown in Scheme I the appropriate 4-substituted quinazoline 2 wherein X is a suitable displaceable leaving group such as halo, aryloxy, alkylsulfinyl, alkylsulfonyl such as trifluoromethanesulfonyloxy, arylsulfinyl, arylsulfonyl, siloxy, cyano, pyrazolo, triazolo or tetrazolo, preferably a 4-chloroquinazoline, is reacted with the appropriate amine or amine hydrochloride 4 or 5, wherein R⁴ is as described above and Y is Br, I, or trifluoromethane-sulfonyloxy in a solvent such as a (C₁-C₆)alcohol, dimethylformamide (DMF), N-methylpyrrolidin-2-one, chloroform, acetonitrile, tetrahydrofuran (THF), 1-4 dioxane, pyridine or other aprotic solvent. The reaction may be effected in the presence of a base, preferably an alkali or alkaline earth metal carbonate or hydroxide or a tertiary amine base, such as pyridine, 2,6-lutidine, collidine, N-methyl-morpholine, triethylamine, 4-dimethylamino-pyridine or N,N-dimethylaniline. These bases are hereinafter referred to as suitable bases. The reaction mixture is maintained at a temperature from about ambient to about the reflux temperature of the solvent, preferably from about 35° C. to about reflux, until substantially no remaining 4-haloquinazoline can be detected, typically about 2 to about 24 hours. Preferably, the reaction is performed under an inert atmosphere such as dry nitrogen.

Generally the reactants are combined stoichiometrically. When an amine base is used for is those compounds where a salt (typically the HCl salt) of an amine 4 or 5 is used, it is preferable to use excess amine base, generally an extra equivalent of amine base. (Alternatively, if an amine base is not used an excess of the amine 4 or 5 may be used).

For those compounds where a sterically hindered amine 4 (such as a 2-alkyl-3-ethynylaniline) or very reactive 4-haloquinazoline is used it is preferable to use t-butyl alcohol or a polar aprotic solvent such as DMF or N-methylpyrrolidin-2-one as the solvent.

Alternatively, a 4-substituted quinazoline 2 wherein X is hydroxyl or oxo (and the 2-nitrogen is hydrogenated) is reacted with carbon tetrachloride and an optionally substituted triarylphosphine which is optionally supported on an inert polymer (e.g. triphenylphosphine, polymer supported, Aldrich Cat. No. 36,645-5, which is a 2% divinylbenzene cross-linked polystyrene containing 3 mmol phosphorous per gram resin) in a solvent such as carbon tetrachloride, chloroform, dichloroethane, tetrahydrofuran, acetonitrile or other aprotic solvent or mixtures thereof. The reaction mixture is maintained at a temperature from about ambient to reflux, preferably from about 35° C. to reflux, for 2 to 24 hours. This mixture is reacted with the appropriate amine or amine hydrochloride 4 or 5 either directly or after removal of solvent, for example by vacuum evaporation, and addition of a suitable alternative solvent such as a (C₁-C₆) alcohol, DMF, N-methylpyrrolidin-2-one, pyridine or 1-4 dioxane. Then, the reaction mixture is maintained at a temperature from about ambient to the reflux temperature of the solvent preferably from about 35° C. to about reflux, until substantially complete formation of product is achieved, typically from about 2 to about 24 hours. Preferably the reaction is performed under an inert atmosphere such as dry nitrogen.

When compound 4, wherein Y is Br, I, or trifluoromethanesulfonyloxy, is used as starting material in the reaction with quinazoline 2, a compound of formula 3 is formed wherein R¹, R², R³, and Y are as described above. Compound 3 is converted to compounds of formula I wherein R⁴ is R¹¹ ethynyl, and R¹¹ is as defined above, by reaction with a suitable palladium reagent such as tetrakis(triphenylphosphine)palladium or bis(triphenylphosphine)palladium dichloride in the presence of a suitable Lewis acid such as cuprous chloride and a suitable alkyne such as trimethylsilylacetylene, propargyl alcohol or 3-(N,N-dimethylamino)-propyne in a solvent such as diethylamine or triethylamine. Compounds 3, wherein Y is NH₂, may be converted to compounds I wherein R⁴ is azide by treatment of compound 3 with a diazotizing agent, such as an acid and a nitrite (e.g., acetic acid and NaNO₂) followed by treatment of the resulting product with an azide, such as NaN₃.

For the production of those compounds of Formula I wherein an R¹ is an amino or hydroxyamino group the reduction of the corresponding Formula I compound wherein R¹ is nitro is employed.

The reduction may conveniently be carried out by any of the many procedures known for such transformations. The reduction may be carried out, for example, by hydrogenation of the nitro compound in a reaction-inert solvent in the presence of a suitable metal catalyst such as palladium, platinum or nickel. A further suitable reducing agent is, for example, an activated metal such as activated iron (produced by washing iron powder with a dilute solution of an acid such as hydrochloric acid). Thus, for example, the reduction may be carried out by heating a mixture of the nitro compound and the activated metal with concentrated hydrochloric acid in a solvent such as a mixture of water and an alcohol, for example, methanol or ethanol, to a temperature in the range, for example, 50° to 150° C., conveniently at or near 70° C. Another suitable class of reducing agents are the alkali metal dithionites, such as sodium dithionite, which may be used in (C₁-C₄)alkanoic acids, (C₁-C₆)alkanols, water or mixtures thereof.

For the production of those compounds of Formula I wherein R² or R³ incorporates a primary or secondary amino moiety (other than the amino group intended to react with the quinazoline), such free amino group is preferably protected prior to the above described reaction followed by deprotection, subsequent to the above described reaction with 4-(substituted)quinazoline 2.

Several well known nitrogen protecting groups can be used. Such groups include (C₁-C₆)alkoxycarbonyl, optionally substituted benzyloxycarbonyl, aryloxycarbonyl, trityl, vinyloxycarbonyl, O-nitrophenylsulfonyl, diphenylphosphinyl, p-toluenesulfonyl, and benzyl. The addition of the nitrogen protecting group may be carried out in a chlorinated hydrocarbon solvent such as methylene chloride or 1,2-dichloroethane, or an ethereal solvent such as glyme, diglyme or THF, in the presence or absence of a tertiary amine base such as triethylamine, diisopropylethylamine or pyridine, preferably triethylamine, at a temperature from about 0° C. to about 50° C., preferably about ambient temperature. Alternatively, the protecting groups are conveniently attached using Schotten-Baumann conditions.

Subsequent to the above described coupling reaction, of compounds 2 and 5, the protecting group may be removed by deprotecting methods known to those skilled in the art such as treatment with trifluoroacetic acid in methylene chloride for the tert-butoxycarbonyl protected products.

For a description of protecting groups and their use, see T. W. Greene and P. G. M. Wuts, “Protective Groups in Organic Synthesis” Second Ed., John Wiley & Sons, New York, 1991.

For the production of compounds of Formula I wherein R¹ or R² is hydroxy, cleavage of a Formula I compound wherein R¹ or R² is (C₁-C₄)alkoxy is preferred.

The cleavage reaction may conveniently be carried out by any of the many procedures known for such a transformation. Treatment of the protected formula I derivative with molten pyridine hydrochloride (20-30 eq.) at 150° to 175° C. may be employed for O-dealkylations. Alternatively, the cleavage reaction may be carried out, for example, by treatment of the protected quinazoline derivative with an alkali metal (C₁-C₄)alkylsulphide, such as sodium ethanethiolate or by treatment with an alkali metal diarylphosphide such as lithium diphenylphosphide. The cleavage reaction may also, conveniently, be carried out by treatment of the protected quinazoline derivative with a boron or aluminum trihalide such as boron tribromide. Such reactions are preferably carried out in the presence of a reaction-inert solvent at a suitable temperature.

Compounds of formula I, wherein R¹ or R² is a (C₁-C₄)alkylsulphinyl or (C₁-C₄)alkylsulphonyl group are preferably prepared by oxidation of a formula I compound wherein R¹ or R² is a (C₁-C₄)alkylsulfanyl group. Suitable oxidizing agents are known in the art for the oxidation of sulfanyl to sulphinyl and/or sulphonyl, e.g., hydrogen peroxide, a peracid (such as 3-chloroperoxybenzoic or peroxyacetic acid), an alkali metal peroxysulphate (such as potassium peroxymonosulphate), chromium trioxide or gaseous oxygen in the presence of platinum. The oxidation is generally carried out under as mild conditions as possible using the stoichiometric amount of oxidizing agent in order to reduce the risk of over oxidation and damage to other functional groups. In general, the reaction is carried out in a suitable solvent such as methylene chloride, chloroform, acetone, tetrahydrofuran or tert-butyl methyl ether and at a temperature from about −25° to 50° C., preferably at or near ambient temperature, i.e., in the range of 15° to 35° C. When a compound carrying a sulphinyl group is desired a milder oxidizing agents should be used such as sodium or potassium metaperiodate, conveniently in a polar solvent such as acetic acid or ethanol. The compounds of formula I containing a (C₁-C₄)alkylsulphonyl group may be obtained by oxidation of the corresponding (C₁-C₄)alkylsulphinyl compound as well as of the corresponding (C₁-C₄)alkylsulfanyl compound.

Compounds of formula I wherein R¹ is optionally substituted (C₂-C₄)alkanoylamino, ureido, 3-phenylureido, benzamido or sulfonamido can be prepared by acylation or sulfonylation of a corresponding compound wherein R¹ is amino. Suitable acylating agents are any agents known in the art for the acylation of amino to acylamino, for example, acyl halides, e.g., a (C₂-C₄)alkanoyl chloride or bromide or a benzoyl chloride or bromide, alkanoic acid anhydrides or mixed anhydrides (e.g., acetic anhydride or the mixed anhydride formed by the reaction of an alkanoic acid and a (C₁-C₄)alkoxycarbonyl halide, for example (C₁-C₄)alkoxycarbonyl chloride, in the presence of a suitable base. For the production of those compounds of Formula I wherein R¹ is ureido or 3-phenylureido, a suitable acylating agent is, for example, a cyanate, e.g., an alkali metal cyanate such as sodium cyanate, or an isocyanate such as phenyl isocyanate. N-sulfonylations may be carried out with suitable sulfonyl halides or sulfonylanhydrides in the presence of a tertiary amine base. In general the acylation or sulfonylation is carried out in a reaction-inert solvent and at a temperature in the range of about −30° to 120° C., conveniently at or near ambient temperature.

Compounds of Formula I wherein R¹ is (C₁-C₄)alkoxy or substituted (C₁-C₄)alkoxy or R¹ is (C₁-C₄)alkylamino or substituted mono-N- or di-N,N—(C₁-C₄)alkylamino, are prepared by the alkylation, preferably in the presence of a suitable base, of a corresponding compound wherein R¹ is hydroxy or amino, respectively. Suitable alkylating agents include alkyl or substituted alkyl halides, for example, an optionally substituted (C₁-C₄)alkyl chloride, bromide or iodide, in the presence of a suitable base in a reaction-inert solvent and at a temperature in the range of about 10° to 140° C., conveniently at or near ambient temperature.

For the production of those compounds of Formula I wherein R¹ is an amino-, oxy- or cyano-substituted (C₁-C₄)alkyl substituent, a corresponding compound wherein R¹ is a (C₁-C₄)alkyl substituent bearing a group which is displaceable by an amino-, alkoxy-, or cyano group is reacted with an appropriate amine, alcohol or cyanide, preferably in the presence of a suitable base. The reaction is preferably carried out in a reaction-inert solvent or diluent and at a temperature in the range of about 10° to 100° C., preferably at or near ambient temperature.

Compounds of Formula I, wherein R¹ is a carboxy substituent or a substituent which includes a carboxy group are prepared by hydrolysis of a corresponding compound wherein R¹ is a (C₁-C₄)alkoxycarbonyl substituent or a substituent which includes a (C₁-C₄)alkoxycarbonyl group. The hydrolysis may conveniently be performed, for example, under basic conditions, e.g., in the presence of alkali metal hydroxide.

Compounds of Formula I wherein R¹ is amino, (C₁-C₄)alkylamino, di-[(C₁-C₄)alkyl]amino, pyrrolidin-1-yl, piperidino, morpholino, piperazin-1-yl, 4-(C₁-C₄)alkylpiperazin-1-yl or (C₁-C₄)alkysulfanyl, may be prepared by the reaction, in the presence of a suitable base, of a corresponding compound wherein R¹ is an amine or thiol displaceable group with an appropriate amine or thiol. The reaction is preferably carried out in a reaction-inert solvent or diluent and at a temperature in the range of about 10° to 180° C., conveniently in the range 100° to 150° C.

Compounds of Formula I wherein R¹ is 2-oxopyrrolidin-1-yl or 2-oxopiperidin-1-yl are prepared by the cyclisation, in the presence of a suitable base, of a corresponding compound wherein R¹ is a halo-(C₂-C₄)alkanoylamino group. The reaction is preferably carried out in a reaction-inert solvent or diluent and at a temperature in the range of about 10° to 100° C., conveniently at or near ambient temperature.

For the production of compounds of Formula I in which R¹ is carbamoyl, substituted carbamoyl, alkanoyloxy or substituted alkanoyloxy, the carbamoylation or acylation of a corresponding compound wherein R¹ is hydroxy is convenient.

Suitable acylating agents known in the art for acylation of hydroxyaryl moieties to alkanoyloxyaryl groups include, for example, (C₂-C₄)alkanoyl halides, (C₂-C₄)alkanoyl anhydrides and mixed anhydrides as described above, and suitable substituted derivatives thereof may be employed, typically in the presence of a suitable base. Alternatively, (C₂-C₄)alkanoic acids or suitably substituted derivatives thereof may be coupled with a Formula I compound wherein R¹ is hydroxy with the aid of a condensing agent such as a carbodiimide. For the production of those compounds of Formula I in which R¹ is carbamoyl or substituted carbamoyl, suitable carbamoylating agents are, for example, cyanates or alkyl or arylisocyanates, typically in the presence of a suitable base. Alternatively, suitable intermediates such as the chloroformate or carbonylimidazolyl derivative of a compound of Formula I in which R¹ is hydroxy may be generated, for example, by treatment of said derivative with phosgene (or a phosgene equivalent) or carbonyidiimidazole. The resulting intermediate may then be reacted with an appropriate amine or substituted amine to produce the desired carbamoyl derivatives.

Compounds of formula I wherein R¹ is aminocarbonyl or a substituted aminocarbonyl can be prepared by the aminolysis of a suitable intermediate in which R¹ is carboxy.

The activation and coupling of formula I compounds wherein R¹ is carboxy may be performed by a variety of methods known to those skilled in the art. Suitable methods include activation of the carboxyl as an acid halide, azide, symmetric or mixed anhydride, or active ester of appropriate reactivity for coupling with the desired amine. Examples of such types of intermediates and their production and use in couplings with amines may be found extensively in the literature; for example M. Bodansky and A. Bodansky, “The Practice of Peptide Synthesis”, Springer-Verlag, New York, 1984. The resulting formula I compounds may be isolated and purified by standard methods, such as solvent removal and recrystallization or chromatography.

The starting materials for the described reaction scheme I (e.g., amines, quinazolines and amine protecting groups) are readily available or can be easily synthesized by those skilled in the art using conventional methods of organic synthesis. For example, the preparation of 2,3-dihydro-1,4-benzoxazine derivatives are described in R. C. Elderfield, W. H. Todd, S. Gerber, Ch. 12 in “Heterocyclic Compounds”, Vol. 6, R. C. Elderfield ed., John Wiley and Sons, Inc., N.Y., 1957. Substituted 2,3-dihydrobenzothiazinyl compounds are described by R. C. Elderfield and E. E. Harris in Ch. 13 of Volume 6 of the Elderfield “Heterocyclic Compounds” book.

In another particular embodiment, the EGFR antagonist has a general formula II as described in U.S. Pat. No. 5,457,105, incorporated herein by reference:

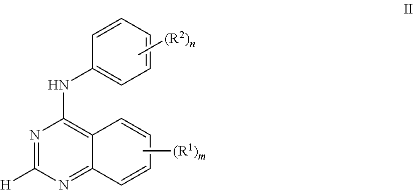

wherein:

m is 1, 2 or 3 and

each R¹ is independently 6-hydroxy, 7-hydroxy, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkoxyamino, (2-4C)alkanoyloxyamino, trifluoromethoxy, (1-4C)alkyl, 6-(1-4C)alkoxy, 7-(1-4C)alkoxy, (1-3C)alkylenedioxy, (1-4C)alkylamino, di-1[(1-4C)alkyl]amino, pyrrolidin-1-yl, piperidino, morpholino, piperazin-1-yl, 4-(1-4C)alkylpiperazin-1-yl, (1-4C)alkylthio, (1-4C)alkylsulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, carboxy-(1-4C)alkyl, (1-4C)alkoxycarbonyl-(1-4C)alkyl, carbamoyl-(1-4C)alkyl, N-(1-4C)alkylcarbamoyl-(1-4C)alkyl, N, N-di-[(1-4C)alkyl]carbamoyl-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C) alkyl, 4-(1-4C)alkylpiperazin-1-yl-(1-4C) alkyl, hydroxy-(2-4C)alkoxy-(1-4C) alkyl, (1-4C)alkoxy-(2-4C)alkoxy-(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, phenoxy-(1-4C)alkyl, anilino-(1-4C)alkyl, phenylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C) alkylcarbamoyl-(1-4C)alkoxy, N, N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, (2-4C)alkanoyloxy, hydroxy-(2-4C)alkanoyloxy, (1-4C)alkoxy-(2-4C)alkanoyloxy, phenyl-(1-4C)alkoxy, phenoxy-(2-4C)alkoxy, anilino-(2-4C)alkoxy, phenylthio-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)akylamino, carboxy-(1-4C)alkylamino, (1-4C)alkoxycarbonyl-(1-4C)alkylamino, carbamoyl-(1-4C)alkylamino, N-(1-4C)alkylcarbamoyl-(1-4C)alkylamino, N,N-di-[(1-4C)alkyl]carbamoyl-(1-4C)alkylamino, amino-(2-4C)alkylamino, (1-4C)alkylamino-(2-4C)alkylamino, di-1(1-4C)alkyl]amino-(2-4C)alkylamino, phenyl-(1-4C)alkylamino, phenoxy-(2-4C)alkylamino, anilino-(2-4C)alkylamino, phenylthio-(2-4C)alkylamino, (2-4C)alkanoylamino, (1-4C)alkoxycarbonylamino, (1-4C)alkylsulphonylamino, benzamido, benzenesulphonamido, 3-phenylureido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, carbamoyl-(2-4C)alkanoylamino, N-(1-4C)alkylcarbamoyl-(2-4C)alkanoylamino, N,N-di-[(1-4C)alkyl]carbamoyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent or any anilino, phenoxy or phenyl group in a R¹ substituent may optionally bear one or two halogeno, (1-4C)alkyl or (1-4C)alkoxy substituents;

n is 1 or 2 and

each R² is independently hydrogen, hydroxy, halogeno, trifluoromethyl, amino, nitro, cyano, (1-4C)alkyl, (1-4C)alkoxy, (1-4C)alkylamino, di-[(1-4C)alkyl]amino, (1-4C)alkylthio, (1-4C)alkylsulphinyl or(1-4C)alkylsulphonyl; or a pharmaceutically-acceptable salt thereof; except that 4-(4′-hydroxyanilino)-6-methoxyquinazoline, 4-(4,-hydroxyanilino)-6,7-methylenedioxyquinazoline, 6-amino-4-(4′-aminoanilino)quinazoline, 4-anilino-6-methylquinazoline or the hydrochloride salt thereof and 4-anilino-6,7-dimethoxyquinazoline or the hydrochloride salt thereof are excluded.

In a particular embodiment, the EGFR antagonist is a compound according to formula II selected from the group consisting of: 4-(3′-chloro-4′-fluoroanilino)-6,7-dimethoxyquinazoline; 4-(3′,4′-dichloroanilino)-6,7-dimethoxyquinazoline; 6,7-dimethoxy-4-(3′-nitroanilino)-quinazoline; 6,7-diethoxy-4-(3′-methylanilino)-quinazoline; 6-methoxy-4-(3′-methylanilino)-quinazoline; 4-(3′-chloroanilino)-6-methoxyquinazoline; 6,7-ethylenedioxy-4-(3′-methylanilino)-quinazoline; 6-amino-7-methoxy-4-(3′-methylanilino)-quinazoline; 4-(3′-methylanilino)-6-ureidoquinazoline; 6-(2-methoxyethoxymethyl)-4-(3′-methylanilino)-quinazoline; 6,7-di-(2-methoxyethoxy)-4-(3′-methylanilino)-quinazoline; 6-dimethylamino-4-(3′-methylanilino)quinazoline; 6-benzamido-4-(3′-methylanilino)quinazoline; 6,7-dimethoxy-4-(3′-trifluoromethylanilino)-quinazoline; 6-hydroxy-7-methoxy-4-(3′-methylanilino)-quinazoline; 7-hydroxy-6-methoxy-4-(3′-methylanilino)-quinazoline; 7-amino-4-(3′-methylanilino)-quinazoline; 6-amino-4-(3′-methylanilino)quinazoline; 6-amino-4-(3′-chloroanilino)-quinazoline; 6-acetamido-4-(3′-methylanilino)-quinazoline; 6-(2-methoxyethylamino)-4-(3′-methylanilino)-quinazoline; 7-(2-methoxyacetamido)-4-(3′-methylanilino)-quinazoline; 7-(2-hydroxyethoxy)-6-methoxy-4-(3′-methylanilino)-quinazoline; 7-(2-methoxyethoxy)-6-methoxy-4-(3′-methylanilino)-quinazoline; 6-amino-4-(3′-methylanilino)-quinazoline.

A quinazoline derivative of the formula II, or a pharmaceutically-acceptable salt thereof, may be prepared by any process known to be applicable to the preparation of chemically-related compounds. A suitable process is, for example, illustrated by that used in U.S. Pat. No. 4,322,420. Necessary starting materials may be commercially available or obtained by standard procedures of organic chemistry.

(a) The reaction, conveniently in the presence of a suitable base, of a quinazoline (i), wherein Z is a displaceable group, with an aniline (ii).

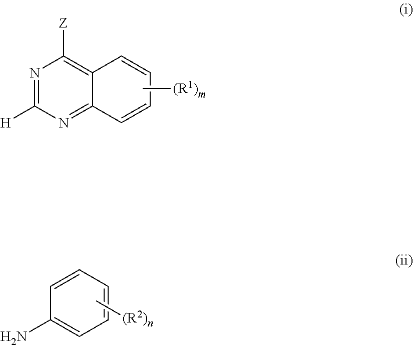

A suitable displaceable group Z is, for example, a halogeno, alkoxy, aryloxy or sulphonyloxy group, for example a chloro, bromo, methoxy, phenoxy, methanesulphonyloxy or toluene-p-sulphonyloxy group.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or for example, an alkali or alkaline earth metal carbonate or hydroxide, for example sodium carbonate, potassium carbonate, calcium carbonate, sodium hydroxide or potassium hydroxide.

The reaction is preferably carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 10° to 150° C., preferably in the range 20° to 80° C.

The quinazoline derivative of the formula II may be obtained from this process in the form of the free base or alternatively it may be obtained in the form of a salt with the acid of the formula H—Z wherein Z has the meaning defined hereinbefore. When it is desired to obtain the free base from the salt, the salt may be treated with a suitable base as defined hereinbefore using a conventional procedure.

(b) For the production of those compounds of the formula II wherein R¹ or R² is hydroxy, the cleavage of a quinazoline derivative of the formula II wherein R¹ or R² is (1-4C)alkoxy.

The cleavage reaction may conveniently be carried out by any of the many procedures known for such a transformation. The reaction may be carried out, for example, by treatment of the quinazoline derivative with an alkali metal (1-4C)alkylsulphide such as sodium ethanethiolate or, for example, by treatment with an alkali metal diarylphosphide such as lithium diphenylphosphide. Alternatively the cleavage reaction may conveniently be carried out, for example, by treatment of the quinazoline derivative with a boron or aluminium trihalide such as boron tribromide. Such reactions are preferably carried out in the presence of a suitable inert solvent or diluent as defined hereinbefore and at a suitable temperature.

(c) For the production of those compounds of the formula II wherein R¹ or R² is a (1-4C)alkylsulphinyl or (1-4C)alkylsulphonyl group, the oxidation of a quinazoline derivative of the formula II wherein R¹ or R² is a (1-4C)alkylthio group.

A suitable oxidising agent is, for example, any agent known in the art for the oxidation of thio to sulphinyl and/or sulphonyl, for example, hydrogen peroxide, a peracid (such as 3-chloroperoxybenzoic or peroxyacetic acid), an alkali metal peroxysulphate (such as potassium peroxymonosulphate), chromium trioxide or gaseous oxygen in the presence of platinium. The oxidation is generally carried out under as mild conditions as possible and with the required stoichiometric amount of oxidising agent in order to reduce the risk of over oxidation and damage to other functional groups. In general the reaction is carried out in a suitable solvent or diluent such as methylene chloride, chloroform, acetone, tetrahydrofuran or tert-butyl methyl ether and at a temperature, for example, −25° to 50° C., conveniently at or near ambient temperature, that is in the range 15° to 35° C. When a compound carrying a sulphinyl group is required a milder oxidising agent may also be used, for example sodium or potassium metaperiodate, conveniently in a polar solvent such as acetic acid or ethanol. It will be appreciated that when a compound of the formula II containing a (1-4C)alkylsulphonyl group is required, it may be obtained by oxidation of the corresponding (1-4C)alkylsulphinyl compound as well as of the corresponding (1-4C)alkylthio compound.

(d) For the production of those compounds of the formula II wherein R¹ is amino, the reduction of a quinazoline derivative of the formula I wherein R¹ is nitro.

The reduction may conveniently be carried out by any of the many procedures known for such a transformation. The reduction may be carried out, for example, by the hydrogenation of a solution of the nitro compound in an inert solvent or diluent as defined hereinbefore in the presence of a suitable metal catalyst such as palladium or platinum. A further suitable reducing agent is, for example, an activated metal such as activated iron (produced by washing iron powder with a dilute solution of an acid such as hydrochloric acid). Thus, for example, the reduction may be carried out by heating a mixture of the nitro compound and the activated metal in a suitable solvent or diluent such as a mixture of water and an alcohol, for example, methanol or ethanol, to a temperature in the range, for example, 50° to 150° C., conveniently at or near 70° C.

(e) For the production of those compounds of the formula II wherein R¹ is (2-4C)alkanoylamino or substituted (2-4C)alkanoylamino, ureido, 3-phenylureido or benzamido, or R² is acetamido or benzamido, the acylation of a quinazoline derivative of the formula II wherein R¹ or R² is amino.

A suitable acylating agent is, for example, any agent known in the art for the acylation of amino to acylamino, for example an acyl halide, for example a (2-4C)alkanoyl chloride or bromide or a benzoyl chloride or bromide, conveniently in the presence of a suitable base, as defined hereinbefore, an alkanoic acid anhydride or mixed anhydride, for example a (2-4C)alkanoic acid anhydride such as acetic anhydride or the mixed anhydride formed by the reaction of an alkanoic acid and a (1-4C)alkoxycarbonyl halide, for example a (1-4C)alkoxycarbonyl chloride, in the presence of a suitable base as defined hereinbefore. For the production of those compounds of the formula II wherein R¹ is ureido or 3-phenylureido, a suitable acylating agent is, for example, a cyanate, for example an alkali metal cyanate such as sodium cyanate or, for example, an isocyanate such as phenyl isocyanate. In general the acylation is carried out in a suitable inert solvent or diluent as defined hereinbefore and at a temperature, in the range, for example, −30° to 120° C., conveniently at or near ambient temperature.

(f) For the production of those compounds of the formula II wherein R¹ is (1-4C)alkoxy or substituted (1-4C)alkoxy or R¹ is (1-4C)alkylamino or substituted (1-4C)alkylamino, the alkylation, preferably in the presence of a suitable base as defined hereinbefore, of a quinazoline derivative of the formula II wherein R¹ is hydroxy or amino as appropriate.

A suitable alkylating agent is, for example, any agent known in the art for the alkylation of hydroxy to alkoxy or substituted alkoxy, or for the alkylation of amino to alkylamino or substituted alkylamino, for example an alkyl or substituted alkyl halide, for example a (1-4C)alkyl chloride, bromide or iodide or a substituted (1-4C)alkyl chloride, bromide or iodide, in the presence of a suitable base as defined hereinbefore, in a suitable inert solvent or diluent as defined hereinbefore and at a temperature in the range, for example, 10° to 140° C., conveniently at or near ambient temperature.

(g) For the production of those compounds of the formula II wherein R¹ is a carboxy substituent or a substituent which includes a carboxy group, the hydrolysis of a quinazoline derivative of the formula II wherein R¹ is a (1-4C)alkoxycarbonyl substituent or a substituent which includes a (1-4C)alkoxycarbonyl group.

The hydrolysis may conveniently be performed, for example, under basic conditions.

(h) For the production of those compounds of the formula II wherein R¹ is an amino-, oxy-, thio- or cyano-substituted (1-4C)alkyl substituent, the reaction, preferably in the presence of a suitable base as defined hereinbefore, of a quinazoline derivative of the formula II wherein R¹ is a (1-4C)alkyl substituent bearing a displaceable group as defined hereinbefore with an appropriate amine, alcohol, thiol or cyanide.

The reaction is preferably carried out in a suitable inert solvent or diluent as defined hereinbefore and at a temperature in the range, for example, 10° to 100° C., conveniently at or near ambient temperature.

When a pharmaceutically-acceptable salt of a quinazoline derivative of the formula II is required, it may be obtained, for example, by reaction of said compound with, for example, a suitable acid using a conventional procedure.

In a particular embodiment, the EGFR antagonist is a compound according to formula II′ as disclosed in U.S. Pat. No. 5,770,599, incorporated herein by reference:

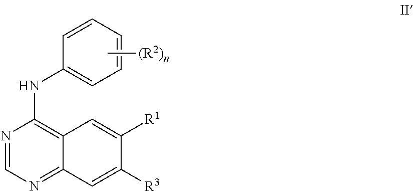

wherein:

n is 1, 2 or 3;

each R² is independently halogeno or trifluoromethyl

R³ is (1-4C)alkoxy; and

R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy, and wherein any of the above mentioned R¹ substituents comprising a CH₂ (methylene) group which is not attached to a N or O atom optionally bears on said CH₂ group a hydroxy substituent;

or a pharmaceutically-acceptable salt thereof.

In a particular embodiment, the EGFR antagonist is a compound according to formula II′ selected from the group consisting of: 4-(3′-chloro-4′-fluoroanilino)-7-methoxy-6-(2-pyrrolidin-1-ylethoxy)-quinazoline; 4-(3′-chloro-4′-fluoroanilino)-7-methoxy-6-(2-morpholinoethoxy)-quinazoline; 4-(3′-chloro-4′-fluoroanilino)-6-(3-diethylaminopropoxy)-7-methoxyquinazoline; 4-(3′-chloro-4′-fluoroanilino)-7-methoxy-6-(3-pyrrolidin-1-ylpropoxy)-quinazoline; 4-(3′-chloro-4′-fluoroanilino)-6-(3-dimethylaminopropoxy)-7-methoxyquinazoline; 4-(3′,4′-difluroanilino)-7-methoxy-6-(3-morpholinopropoxy)-quinazoline; 4-(3′-chloro-4′-fluoroanilino)-7-methoxy-6-(3-piperidinopropoxy)-quinazoline; 4-(3′-chloro-4′-fluoroanilino)-7-methoxy-6-(3-morpholinopropoxy)-quinazoline; 4-(3′-chloro-4′-fluoroanilino)-6-(2-dimethylaminoethoxy)-7-methoxyquinazoline; 4-(2′,4′-difluoroanilino)-6-(3-dimethylaminopropoxy)-7-methoxyquinazoline; 4-(2′,4′-difluoroanilino)-7-methoxy-6-(3-morpholinopropoxy)-quinazoline, 4-(3′-chloro-4′-fluoroanilino)-6-(2-imidazol-1-ylethoxy)-7-methoxyquinazoline; 4-(3′-chloro-4′-fluoroanilino)-6-(3-imidazol-1-ylpropoxy)-7-methoxyquinazoline; 4-(3′-chloro-4′-fluoroanilino)-6-(2-dimethylaminoethoxy)-7-methoxyquinazoline; 4-(2′,4′-difluoroanilino)-6-(3-dimethylaminopropoxy)-7-methoxyquinazoline; 4-(2′,4′-difluoroanilino)-7-methoxy-6-(3-morpholinopropoxy)-quinazoline; 4-(3′-chloro-4′-fluoroanilino)-6-(2-imidazol-1-ylethoxy)-7-methoxyquinazoline; and 4-(3′-chloro-4′-fluoroanilino)-6-(3-imidazol-1-ylpropoxy)-7-methoxyquinazoline.

In a particular embodiment, the EGFR antagonist is a compound according to formula II′ that is 4-(3′-chloro-4′-fluoroanilino)-7-methoxy-6-(3-morpholinopropoxy)-quinazoline, alternatively referred to as ZD 1839, gefitinib and Iressa®.

A quinazoline derivative of the formula II′, or a pharmaceutically-acceptable salt thereof, may be prepared by any process known to be applicable to the preparation of chemically-related compounds. Suitable processes include, for example, those illustrated in U.S. Pat. No. 5,616,582, U.S. Pat. No. 5,580,870, U.S. Pat. No. 5,475,001 and U.S. Pat. No. 5,569,658. Unless otherwise stated, n, R², R³ and R¹ have any of the meanings defined hereinbefore for a quinazoline derivative of the formula II′. Necessary starting materials may be commercially available or obtained by standard procedures of organic chemistry.

(a) The reaction, conveniently in the presence of a suitable base, of a quinazoline (iii) wherein Z is a displaceable group, with an aniline (iv)

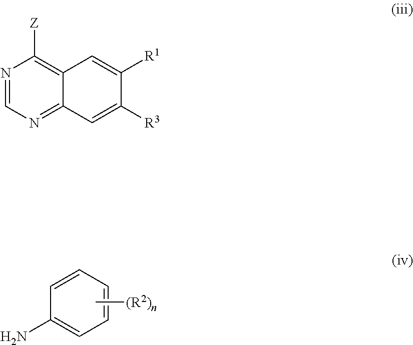

A suitable displaceable group Z is, for example, a halogeno, alkoxy, aryloxy or sulphonyloxy group, for example a chloro, bromo, methoxy, phenoxy, methanesulphonyloxy or toluene-4-sulphonyloxy group.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or for example, an alkali or alkaline earth metal carbonate or hydroxide, for example sodium carbonate, potassium carbonate, calcium carbonate, sodium hydroxide or potassium hydroxide. Alternatively a suitable base is, for example, an alkali metal or alkaline earth metal amide, for example sodium amide or sodium bis(trimethylsilyl)amide.

The reaction is preferably carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 10° to 150° C., preferably in the range 20° to 80° C.

The quinazoline derivative of the formula II′ may be obtained from this process in the form of the free base or alternatively it may be obtained in the form of a salt with the acid of the formula H—Z wherein Z has the meaning defined hereinbefore. When it is desired to obtain the free base from the salt, the salt may be treated with a suitable base as defined hereinbefore using a conventional procedure.

(b) For the production of those compounds of the formula II′ wherein R¹ is an amino-substituted (2-4C)alkoxy group, the alkylation, conveniently in the presence of a suitable base as defined hereinbefore, of a quinazoline derivative of the formula II′ wherein R¹ is a hydroxy group.

A suitable alkylating agent is, for example, any agent known in the art for the alkylation of hydroxy to amino-substituted alkoxy, for example an amino-substituted alkyl halide, for example an amino-substituted (2-4C)alkyl chloride, bromide or iodide, in the presence of a suitable base as defined hereinbefore, in a suitable inert solvent or diluent as defined hereinbefore and at a temperature in the range, for example, 10° to 140° C., conveniently at or near 80° C.

(c) For the production of those compounds of the formula II′ wherein R¹ is an amino-substituted (2-4C)alkoxy group, the reaction, conveniently in the presence of a suitable base as defined hereinbefore, of a compound of the formula II′ wherein R¹ is a hydroxy-(2-4C)alkoxy group, or a reactive derivative thereof, with an appropriate amine.

A suitable reactive derivative of a compound of the formula II′ wherein R¹ is a hydroxy-(2-4C)alkoxy group is, for example, a halogeno- or sulphonyloxy-(2-4C)alkoxy group such as a bromo- or methanesulphonyloxy-(2-4C)alkoxy group.

The reaction is preferably carried out in the presence of a suitable inert solvent or diluent as defined hereinbefore and at a temperature in the range, for example, 10° to 150° C., conveniently at or near 50° C.

(d) For the production of those compounds of the formula II′ wherein R¹ is a hydroxy-amino-(2-4C)alkoxy group, the reaction of a compound of the formula II′ wherein R¹ is a 2,3-epoxypropoxy or 3,4-epoxybutoxy group with an appropriate amine.

The reaction is preferably carried out in the presence of a suitable inert solvent or diluent as defined hereinbefore and at a temperature in the range, for example, 10° to 150° C., conveniently at or near 70° C.

When a pharmaceutically-acceptable salt of a quinazoline derivative of the formula II′ is required, for example a mono- or di-acid-addition salt of a quinazoline derivative of the formula II′, it may be obtained, for example, by reaction of said compound with, for example, a suitable acid using a conventional procedure.

In a particular embodiment, the EGFR antagonist is a compound according to formula III as disclosed in WO9935146, incorporated herein by reference:

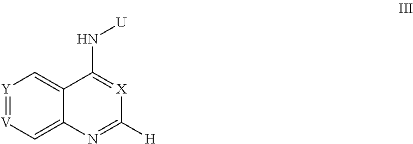

or a salt or solvate thereof; wherein

X is N or CH;

Y is CR¹ and V is N;

or Y is N and V is CR¹;

or Y is CR¹ and V is CR²;

or Y is CR² and V is CR¹;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄alkyl or C₁₋₄alkoxy groups;

R² is selected from the group comprising hydrogen, halo, hydroxy, C₁₋₄alkyl, C₁₋₄alkoxy, C₁₋₄alkylamino and di[C₁₋₄alkyl]amino;

U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;

R³ is selected from a group comprising benzyl, halo-, dihalo- and trihalobenzyl, benzoyl, pyridyimethyl, pyridylmethoxy, phenoxy, benzyloxy, halo-, dihalo- and trihaoobenzyloxy and benzenesulphonyl; or R³ represents trihalomethylbenzyl or trihalomethylbenzyloxy;

or R³ represents a group of formula

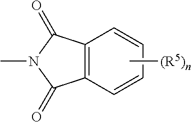

wherein each R⁵ is independently selected from halogen, C₁₋₄alkyl and C₁₋₄alkoxy; and n is O to 3; and

each R⁴ is independently hydroxy, halogen, C₁₋₄alkyl, C₂₋₄alkenyl, C2-4alkynyl, C₁₋₄alkoxy, amino, C₁₋₄alkylamino, di[C₁₋₄alkyl]amino, C1-4alkylthio, C1-4alkylsulphinyl, C₁₋₄alkylsulphonyl, C₁₋₄alkylcarbonyl, carboxy, carbamoyl, C₁₋₄alkoxycarbonyl, C₁₋₄ alkanoylamino, N—(C₁₋₄alkyl)carbamoyl, N,N-di(C₁₋₄alkyl)carbamoyl, cyano, nitro and trifluoromethyl.

In a particular embodiment, EGFR antagonists of formula III exclude: (1-Benzyl-1H-indazol-5-yl)-(6-(5-((2-methanesulphonyl-ethylamino)-methyl)-furan-2-yl)-pyrido[3,4-d]pyrimidin-4-yl-amine; (4-Benzyloxy-phenyl)-(6-(5-((2-methanesulphonyl-ethylamino)-methyl)-furan-2-yl)-pyrido[3,4-d]pyrimidin-4-yl-amine; (1-Benzyl-1H-indazol-5-yl)-(6-(5-((2-methanesulphonyl-ethylamino)-methyl)-furan-2-yl)-quinazolin-4-yl-amine; (1-Benzyl H-indazol-5-yl)-(7-(5-((2-methanesulphonyl-ethylamino)-methyl)-furan-2-yl)-quinazolin-4-yl-amine; and (1-Benzyl-1H-indazol-5-yl)-(6-(5-((2-methanesulphonyl-ethylamino)-methyl)-1-methyl-pyrrol-2-yl)-quinazolin-4-yl-amine.

In a particular embodiment, the EGFR antagonist of formula III are selected from the group consisting of: 4-(4-Fluorobenzyloxy)-phenyl)-(6-(5-((2-methanesulphonyl-ethylamino)methyl)-furan-2-yl)-pyrido[3,4-d]pyrimidin-4-yl)-amine; (4-(3-Fluorobenzyloxy)-phenyl)-(6-(5-((2-methanesulphonyl-ethylamino)methyl)furan-2-yl)-pyrido[3,4-d]pyrimidin-4-yl)-amine; (4-Benzenesulphonyl-phenyl)-(6-(5-((2-methanesulphonyl-ethylamino)-methyl)-furan-2-yl)-pyrido[3,4-d] pyrimidin-4-yl)-amine; (4-Benzyloxy-phenyl)-(6-(3-((2-methanesulphonyl-ethylamino)-methyl)-phenyl)-pyrido[3,4-d]pyrimidin-4-yl)-amine; (4-Benzyloxy-phenyl)-(6-(5-((2-methanesulphonyl-ethylamino)-methyl)-furan-2-yl)quinazolin-4-yl)-amine; (4-(3-Fluorobenzyloxy-phenyl)-(6-(4-((2-methanesulphonyl-ethylamino)-methyl)-furan-2-yl)-pyrido[3,4-d]pyrimidin-4-yl)-amine; (4-Benzyloxy-phenyl)-(6-(2-((2-methanesulphonylethylamino)-methyl)-thiazol-4-yl)quinazolin-4-yl)-amine; N-{4-[(3-Fluorobenzyl)oxy]phenyl}-6-[5-({[2-(methanesulphonyl)ethyl]amino}methyl)-2-furyl]-4-quinazolinamine; N-{4-[(3-Fluorobenzyl)oxy]-3-methoxyphenyl}-6-[5-({[2-(methanesulphonyl)ethyl]amino}methyl)-2-furyl]-4-quinazolinamine; N-[4-(Benzyloxy)phenyl]-7-methoxy-6-[5-({[2-(methanesulphonyl)ethyl]amino}methyl)-2-furyl]-4-quinazolinamine; N-[4-(Benzyloxy)phenyl]-6-[4-({[2-(methanesulphonyl)ethyl]amino}methyl)-2-furyl]-4-quinazolinamine; N-{4-[(3-Fluorobenzyl)oxy]-3-methoxyphenyl}-6-[2-({[2-(methanesulphonyl)ethyl]amino}methyl)-1,3-thiazol-4-yl]-4-quinazolinamine; N-{4-[(3-Bromobenzyl)oxy]phenyl}-6-[2-({[2-(methanesulphonyl)ethyl]amino}methyl)-1,3-thiazol-4-yl]-4-quinazolinamine; N-{4-[(3-Fluorobenzyl)oxy]phenyl)-6-[2-({[2-(methanesulphonyl)ethyl]amino}methyl)1,3-thiazol-4-yl]-4-quinazolinamine; N-[4-(Benzyloxy)-3-fluoropheny-1]-6-[2-({[2-(methanesulphonyl)ethyl]amino)methyl)-1,3-thiazol-4-yl]-4-quinazolinamine; N-(1-Benzyl-1H-indazol-5-yl)-7-methoxy-6-[5-({[2-(methanesulphonyl)ethyl]amino)methyl)-2-furyl]-4-quinazolinamine; 6-[5-({[2-(Methanesulphonyl)ethyl]amino)methyl)-2-furyl]-N-(4-{[3-(trifluoromethyl)benzyl]oxy)phenyl)-4-quinazolinamine; N-{3-Fluoro-4-[(3-fluorobenzyl)oxy]phenyl}-6-[5-({[2-(methanesulphonyl)ethyl]amino)methyl)-2-furyl]-4-quinazolinamine; N-{4-[(3-Bromobenzyl)oxy]phenyl)-6-[5-({[2-(methanesulphonyl)ethyl]amino)methyl)-2-furyl]-4-quinazolinamine; N-[4-(Benzyloxy)phenyl]-[3-({[2-(methanesulphonyl)ethyl]amino}methyl)-2-furyl]-4-quinazolinamine; N-[1-(3-Fluorobenzyl)-1H-indazol-5-yl]-6-[2-({[2-(methanesulphonyl)ethyl]amino}methyl)-1,3-thiazol-4-yl]-4-quinazolinamine; 6-[5-({[2-(Methanesulphonyl)ethyl]amino)methyl)-2-furyl]-N-[4-(benzenesulphonyl)phenyl]-4-quinazolinamine; 6-[2-({[2-(Methanesulphonyl)ethyl]amino)methyl)-1,3-thiazol-4-yl]-N-[4-(benzenesulphonyl)phenyl)-4-quinazolinamine; 6-[2-({[2-(Methanesulphonyl)ethyl]amino}methyl)-1,3-thiazol-4-yl]-N-(4-{[3-(trifluoromethyl)benzyl]oxy)phenyl)-4-quinazolinamine; N-{3-fluoro-4-[(3-fluorobenzyl)oxy]phenyl)-6-[2-({[2-(methanesulphonyl)ethyl]amino}methyl)-1,3-thiazol-4-yl]-4-quinazolinamine; N-(1-Benzyl-1H-indazol-5-yl)-6-[2-({[2-(methanesulphonyl)ethyl]amino)methyl)-1,3-thiazol-4-yl]-4-quinazolinamine; N-(3-Fluoro-4-benzyloxyphenyl)-6-[2-({[2-(methanesulphonyl)ethyl]amino)methyl)-1,3-thiazol-4-yl]-4-quinazolinamine; N-(3-Chloro-4-benzyloxyphenyl)-6-[2-({[2-(methanesulphonyl)ethyl]amino)methyl)-1,3-thiazol-4-yl]-4-quinazolinamine; N-{3-Chloro-4-[(3-fluorobenzyl)oxy]phenyl}-6-[5-({[2-(methanesulphonyl)ethyl]amino)methyl)-2-furyl]-4-quinazolinamine; 6-[5-({[2-(Methanesulphonyl)ethyl]amino)methyl)-2-furyl]-7-methoxy-N-(4-benzenesulphonyl)phenyl-4-quinazolinamine; N-[4-(Benzyloxy)phenyl]-7-fluoro-6-[5-({[2-(methanesulphonyl)ethyl]amino)methyl)-2-furyl]-4-quinazolinamine; N-(1-Benzyl-1H-indazol-5-yl)-7-fluoro-6-[5-({[2-(methanesulphonyl)ethyl]amino}methyl)-2-furyl]-4-quinazolinamine; N-[4-(Benzenesulphonyl)phenyl]-7-fluoro-6-[5-({[2-(methanesulphonyl)ethyl]amino}methyl)-2-furyl]-4-quinazolinamine; N-(3-Trifluoromethyl-4-benzyloxyphenyl)-6-[5-({[2-(methanesulphonyl)ethyl]amino)methyl)-4-furyl]-4-quinazolinamine; and salts and solvates thereof.

In a particular embodiment, the EGFR antagonist is: N-[3-chloro-4-[(3-fluorophenyl)methoxy]phenyl]-6-[5-[[[2-(methylsulfonyl)ethyl]amino]methyl]-2-furanyl]-4-quinazolinamine ditosylate salt (lapatinib).

In a particular embodiment, the EGFR antagonist is a compound according to formula IV as disclosed in WO0132651, incorporated herein by reference:

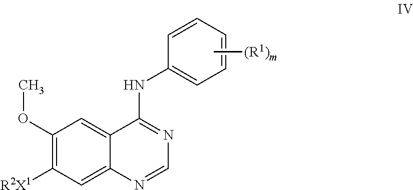

wherein:

m is an integer from 1 to 3;

R¹ represents halogeno or C₁₋₃alkyl;

X¹ represents -0-;

R² is selected from one of the following three groups:

1) C₁₋₅alkylR³ (wherein R³ is piperidin-4-yl which may bear one or two substituents selected from hydroxy, halogeno, C₁₋₄alkyl, C₁₋₄hydroxyalkyl and C₁₋₄alkoxy;

2) C₂₋₅alkenylR³ (wherein R³ is as defined herein);

3) C₂₋₅alkynylR³ (wherein R³ is as defined herein),

and wherein any alkyl, alkenyl or alkynyl group may bear one or more substituents selected from hydroxy, halogeno and amino; or a salt thereof.

In a particular embodiment, the EGFR antagonist is selected from the group consisting of: 4-(4-chloro-2-fluoroanilino)-6-methoxy-7-(1-methylpiperidin-4-ylmethoxy)quinazoline; 4-(2-fluoro-4-methylanilino)-6-methoxy-7-(1-methylpiperidin-4-ylmethoxy)quinazoline; 4-(4-bromo-2-fluoroanilino)-6-methoxy-7-(1-methylpiperidin-4-ylmethoxy)quinazoline, 4-(4-chloro-2,6-difluoroanilino)-6-methoxy-7-(1-methylpiperidin-4-ylmethoxy)quinazoline; 4-(4-bromo-2,6-difluoroanilino)-6-methoxy-7-(1-methylpiperidin-4-ylmethoxy)quinazoline; 4-(4-chloro-2-fluoroanilino)-6-methoxy-7-(piperidin-4-ylmethoxy)quinazoline; 4-(2-fluoro-4-methylanilino)-6-methoxy-7-(piperidin-4-ylmethoxy)quinazoline; 4-(4-bromo-2-fluoroanilino)-6-methoxy-7-(piperidin-4-ylmethoxy)quinazoline; 4-(4-chloro-2,6-difluoroanilino)-6-methoxy-7-(piperidin-4-ylmethoxy)quinazoline; 4-(4-bromo-2,6-difluoroanilino)-6-methoxy-7-(piperidin-4-ylmethoxy)quinazoline; and pharmaceutically acceptable salts and solvates thereof.

In a particular embodiment, the EGFR antagonist is 4-(4-bromo-2-fluoroanilino)-6-methoxy-7-(1-methylpiperidin-4-ylmethoxy)quinazoline (Zactima) and salts thereof.

### Combination Therapies

The present invention features the combination use of a c-met antagonist and an EGFR antagonist as part of a specific treatment regimen intended to provide a beneficial effect from the combined activity of these therapeutic agents. The beneficial effect of the combination includes, but is not limited to, pharmacokinetic or pharmacodynamic co-action resulting from the combination of therapeutic agents. The present invention is particularly useful in treating cancers of various types at various stages.

The term cancer embraces a collection of proliferative disorders, including but not limited to pre-cancerous growths, benign tumors, and malignant tumors. Benign tumors remain localized at the site of origin and do not have the capacity to infiltrate, invade, or metastasize to distant sites. Malignant tumors will invade and damage other tissues around them. They can also gain the ability to break off from the original site and spread to other parts of the body (metastasize), usually through the bloodstream or through the lymphatic system where the lymph nodes are located. Primary tumors are classified by the type of tissue from which they arise; metastatic tumors are classified by the tissue type from which the cancer cells are derived. Over time, the cells of a malignant tumor become more abnormal and appear less like normal cells. This change in the appearance of cancer cells is called the tumor grade, and cancer cells are described as being well-differentiated (low grade), moderately-differentiated, poorly-differentiated, or undifferentiated (high grade). Well-differentiated cells are quite normal appearing and resemble the normal cells from which they originated. Undifferentiated cells are cells that have become so abnormal that it is no longer possible to determine the origin of the cells.

Cancer staging systems describe how far the cancer has spread anatomically and attempt to put patients with similar prognosis and treatment in the same staging group. Several tests may be performed to help stage cancer including biopsy and certain imaging tests such as a chest x-ray, mammogram, bone scan, CT scan, and MRI scan. Blood tests and a clinical evaluation are also used to evaluate a patient's overall health and detect whether the cancer has spread to certain organs.

To stage cancer, the American Joint Committee on Cancer first places the cancer, particularly solid tumors, in a letter category using the TNM classification system. Cancers are designated the letter T (tumor size), N (palpable nodes), and/or M (metastases). T1, T2, T3, and T4 describe the increasing size of the primary lesion; N0, N1, N2, N3 indicates progressively advancing node involvement; and M0 and M1 reflect the absence or presence of distant metastases.

In the second staging method, also known as the Overall Stage Grouping or Roman Numeral Staging, cancers are divided into stages 0 to IV, incorporating the size of primary lesions as well as the presence of nodal spread and of distant metastases. In this system, cases are grouped into four stages denoted by Roman numerals I through IV, or are classified as “recurrent.” For some cancers, stage 0 is referred to as “in situ” or “Tis,” such as ductal carcinoma in situ or lobular carcinoma in situ for breast cancers. High grade adenomas can also be classified as stage 0. In general, stage I cancers are small localized cancers that are usually curable, while stage IV usually represents inoperable or metastatic cancer. Stage II and III cancers are usually locally advanced and/or exhibit involvement of local lymph nodes. In general, the higher stage numbers indicate more extensive disease, including greater tumor size and/or spread of the cancer to nearby lymph nodes and/or organs adjacent to the primary tumor. These stages are defined precisely, but the definition is different for each kind of cancer and is known to the skilled artisan.

Many cancer registries, such as the NCI's Surveillance, Epidemiology, and End Results Program (SEER), use summary staging. This system is used for all types of cancer. It groups cancer cases into five main categories:

In situ is early cancer that is present only in the layer of cells in which it began.

Localized is cancer that is limited to the organ in which it began, without evidence of spread.

Regional is cancer that has spread beyond the original (primary) site to nearby lymph nodes or organs and tissues.

Distant is cancer that has spread from the primary site to distant organs or distant lymph nodes.

Unknown is used to describe cases for which there is not enough information to indicate a stage.

In addition, it is common for cancer to return months or years after the primary tumor has been removed. Cancer that recurs after all visible tumor has been eradicated, is called recurrent disease. Disease that recurs in the area of the primary tumor is locally recurrent, and disease that recurs as metastases is referred to as a distant recurrence.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors. Other examples of cancers are provided in the Definitions.

In some embodiments, the patient herein is subjected to a diagnostic test e.g., prior to and/or during and/or after therapy. Generally, if a diagnostic test is performed, a sample may be obtained from a patient in need of therapy. Where the subject has cancer, the sample may be a tumor sample, or other biological sample, such as a biological fluid, including, without limitation, blood, urine, saliva, ascites fluid, or derivatives such as blood serum and blood plasma, and the like.

The biological sample herein may be a fixed sample, e.g. a formalin fixed, paraffin-embedded (FFPE) sample, or a frozen sample.

Various methods for determining expression of mRNA or protein include, but are not limited to, gene expression profiling, polymerase chain reaction (PCR) including quantitative real time PCR (qRT-PCR), microarray analysis, serial analysis of gene expression (SAGE), MassARRAY, Gene Expression Analysis by Massively Parallel Signature Sequencing (MPSS), proteomics, immunohistochemistry (IHC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR). In one embodiment, expression of one or more of the above noted genes is deemed positive expression if it is at the median or above, e.g. compared to other samples of the same tumor-type. The median expression level can be determined essentially contemporaneously with measuring gene expression, or may have been determined previously.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR. Finally, the data are analyzed to identify the best treatment option(s) available to the patient on the basis of the characteristic gene expression pattern identified in the tumor sample examined.

Detection of gene or protein expression may be determined directly or indirectly.

One may determine expression or amplification of c-met and/or EGFR in the cancer (directly or indirectly). Various diagnostic/prognostic assays are available for this. In one embodiment, c-met and/or EGFR overexpression may be analyzed by IHC. Parafin embedded tissue sections from a tumor biopsy may be subjected to the IHC assay and accorded a c-met and/or EGFR protein staining intensity criteria as follows:

Score 0 no staining is observed or membrane staining is observed in less than 10% of tumor cells.

Score 1+ a faint/barely perceptible membrane staining is detected in more than 10% of the tumor cells. The cells are only stained in part of their membrane.

Score 2+ a weak to moderate complete membrane staining is observed in more than 10% of the tumor cells.

Score 3+ a moderate to strong complete membrane staining is observed in more than 10% of the tumor cells.

In some embodiments, those tumors with 0 or 1+ scores for c-met and/or EGFR overexpression assessment may be characterized as not overexpressing c-met and/or EGFR, whereas those tumors with 2+ or 3+ scores may be characterized as overexpressing c-met and/or EGFR.

In some embodiments, tumors overexpressing c-met and/or EGFR may be rated by immunohistochemical scores corresponding to the number of copies of c-met and/or EGFR molecules expressed per cell, and can been determined biochemically:

0=0-10,000 copies/cell,

1+=at least about 200,000 copies/cell,

2+=at least about 500,000 copies/cell,

3+=at least about 2,000,000 copies/cell.

Alternatively, or additionally, FISH assays may be carried out on formalin-fixed, paraffin-embedded tumor tissue to determine the extent (if any) of c-met and/or EGFR amplification in the tumor.

C-met or EGFR activation may be determined directly (e.g., by phospho-ELISA testing, or other means of detecting phosphorylated receptor) or indirectly (e.g., by detection of activated downstream signaling pathway components, detection of receptor dimmers (e.g., homodimers, heterodimers), detection of gene expression profiles and the like.

Similarly, c-met or EGFR constitutive activation or presence of ligand-independent EGFR or c-met may be detected directly or indirectly (e.g., by detection of receptor mutations correlated with constitutive activity, by detection of receptor amplification correlated with constitutive activity and the like).

Methods for detection of nucleic acid mutations are well known in the art. Often, though not necessarily, a target nucleic acid in a sample is amplified to provide the desired amount of material for determination of whether a mutation is present. Amplification techniques are well known in the art. For example, the amplified product may or may not encompass all of the nucleic acid sequence o encoding the protein of interest, so long as the amplified product comprises the particular amino acid/nucleic acid sequence position where the mutation is suspected to be.

In one example, presence of a mutation can be determined by contacting nucleic acid from a sample with a nucleic acid probe that is capable of specifically hybridizing to nucleic acid encoding a mutated nucleic acid, and detecting said hybridization. In one embodiment, the probe is detectably labeled, for example with a radioisotope (³H, ³²P, ³³P etc), a fluorescent agent (rhodamine, fluorescene etc.) or a chromogenic agent. In some embodiments, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, nucleic acid probes of the invention are provided in a kit for identifying c-met mutations in a sample, said kit comprising an oligonucleotide that specifically hybridizes to or adjacent to a site of mutation in the nucleic acid encoding c-met. The kit may further comprise instructions for treating patients having tumors that contain c-met mutations with a c-met antagonist based on the result of a hybridization test using the kit.

Mutations can also be detected by comparing the electrophoretic mobility of an amplified nucleic acid to the electrophoretic mobility of corresponding nucleic acid encoding wild-type c-met. A difference in the mobility indicates the presence of a mutation in the amplified nucleic acid sequence. Electrophoretic mobility may be determined by any appropriate molecular separation technique, for example on a polyacrylamide gel.

Nucleic acids may also be analyzed for detection of mutations using Enzymatic Mutation Detection (EMD) (Del Tito et al, Clinical Chemistry 44:731-739, 1998). EMD uses the bacteriophage resolvase T₄ endonuclease VII, which scans along double-stranded DNA until it detects and cleaves structural distortions caused by base pair mismatches resulting from nucleic acid alterations such as point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from amplification reactions, eliminating the need for sample purification, shortening the hybridization time, and increasing the signal-to-noise ratio. Mixed samples containing up to a 20-fold excess of normal nucleic acids and fragments up to 4 kb in size can been assayed. However, EMD scanning does not identify particular base changes that occur in mutation positive samples, therefore often requiring additional sequencing procedures to identify the specific mutation if necessary. CEL I enzyme can be used similarly to resolvase T₄ endonuclease VII, as demonstrated in U.S. Pat. No. 5,869,245.

Another simple kit for detecting mutations is a reverse hybridization test strip similar to Haemochromatosis StripAssay™ (Viennalabs http://www.bamburghmarrsh.com/pdf/4220.pdf) for detection of multiple mutations in HFE, TFR2 and FPN1 genes causing Haemochromatosis. Such an assay is based on sequence specific hybridization following amplification by PCR. For single mutation assays, a microplate-based detection system may be applied, whereas for multi-mutation assays, test strips may be used as “macro-arrays”. Kits may include ready-to use reagents for sample prep, amplification and mutation detection. Multiplex amplification protocols provide convenience and allow testing of samples with very limited volumes. Using the straightforward StripAssay format, testing for twenty and more mutations may be completed in less than five hours without costly equipment. DNA is isolated from a sample and the target nucleic acid is amplified in vitro (e.g., by PCR) and biotin-labelled, generally in a single (“multiplex”) amplification reaction. The amplification products are then selectively hybridized to oligonucleotide probes (wild-type and mutant specific) immobilized on a solid support such as a test strip in which the probes are immobilized as parallel lines or bands. Bound biotinylated amplicons are detected using streptavidin-alkaline phosphatase and color substrates. Such an assay can detect all or any subset of the mutations of the invention. With respect to a particular mutant probe band, one of three signaling patterns are possible: (i) a band only for wild-type probe which indicates normal nucleic acid sequence, (ii) bands for both wild-type and a mutant probe which indicates heterozygous genotype, and (iii) band only for the mutant probe which indicates homozygous mutant genotype. Accordingly, in one aspect, the invention provides a method of detecting mutations of the invention comprising isolating and/or amplifying a target c-met nucleic acid sequence from a sample, such that the amplification product comprises a ligand, contacting the amplification product with a probe which comprises a detectable binding partner to the ligand and the probe is capable of specifically hydribizing to a mutation of the invention, and then detecting the hybridization of said probe to said amplification product. In one embodiment, the ligand is biotin and the binding partner comprises avidin or streptavidin. In one embodiment, the binding partner comprises steptavidin-alkaline which is detectable with color substrates. In one embodiment, the probes are immobilized for example on a test strip wherein probes complementary to different mutations are separated from one another. Alternatively, the amplified nucleic acid is labelled with a radioisotope in which case the probe need not comprise a detectable label.

Alterations of a wild-type gene encompass all forms of mutations such as insertions, inversions, deletions, and/or point mutations. In one embodiment, the mutations are somatic. Somatic mutations are those which occur only in certain tissues, e.g., in the tumor tissue, and are not inherited in the germ line. Germ line mutations can be found in any of a body's tissues.

A sample comprising a target nucleic acid can be obtained by methods well known in the art, and that are appropriate for the particular type and location of the tumor. Tissue biopsy is often used to obtain a representative piece of tumor tissue. Alternatively, tumor cells can be obtained indirectly in the form of tissues/fluids that are known or thought to contain the tumor cells of interest. For instance, samples of lung cancer lesions may be obtained by resection, bronchoscopy, fine needle aspiration, bronchial brushings, or from sputum, pleural fluid or blood. Mutant genes or gene products can be detected from tumor or from other body samples such as urine, sputum or serum. The same techniques discussed above for detection of mutant target genes or gene products in tumor samples can be applied to other body samples. Cancer cells are sloughed off from tumors and appear in such body samples. By screening such body samples, a simple early diagnosis can be achieved for diseases such as cancer. In addition, the prowess of therapy can be monitored more easily by testing such body samples for mutant target genes or gene products.

Means for enriching a tissue preparation for tumor cells are known in the art. For example, the tissue may be isolated from paraffin or cryostat sections. Cancer cells may also be separated from normal cells by flow cytometry or laser capture microdissection. These, as well as other techniques for separating tumor from normal cells, are well known in the art. If the tumor tissue is highly contaminated with normal cells, detection of mutations may be more difficult, although techniques for minimizing contamination and/or false positive/negative results are known, some of which are described hereinbelow. For example, a sample may also be assessed for the presence of a biomarker (including a mutation) known to be associated with a tumor cell of interest but not a corresponding normal cell, or vice versa.

Detection of point mutations in target nucleic acids may be accomplished by molecular cloning of the target nucleic acids and sequencing the nucleic acids using techniques well known in the art. Alternatively, amplification techniques such as the polymerase chain reaction (PCR) can be used to amplify target nucleic acid sequences directly from a genomic DNA preparation from the tumor tissue. The nucleic acid sequence of the amplified sequences can then be determined and mutations identified therefrom. Amplification techniques are well known in the art, e.g., polymerase chain reaction as described in Saiki et al., Science 239:487, 1988; U.S. Pat. Nos. 4,683,203 and 4,683,195.

It should be noted that design and selection of appropriate primers are well established techniques in the art.

The ligase chain reaction, which is known in the art, can also be used to amplify target nucleic acid sequences. See, e.g., Wu et al., Genomics, Vol. 4, pp. 560-569 (1989). In addition, a technique known as allele specific PCR can also be used. See, e.g., Ruano and Kidd, Nucleic Acids Research, Vol. 17, p. 8392, 1989. According to this technique, primers are used which hybridize at their 3′ends to a particular target nucleic acid mutation. If the particular mutation is not present, an amplification product is not observed. Amplification Refractory Mutation System (ARMS) can also be used, as disclosed in European Patent Application Publication No. 0332435, and in Newton et al., Nucleic Acids Research, Vol. 17, p. 7, 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Single stranded conformation polymorphism (SSCP) analysis can also be used to detect base change variants of an allele. See, e.g. Orita et al., Proc. Natl. Acad. Sci. USA Vol. 86, pp. 2766-2770, 1989, and Genomics, Vol. 5, pp. 874-879, 1989. Other techniques for detecting insertions and deletions as known in the art can also be used.

Alteration of wild-type genes can also be detected on the basis of the alteration of a wild-type expression product of the gene. Such expression products include both mRNA as well as the protein product. Point mutations may be detected by amplifying and sequencing the mRNA or via molecular cloning of cDNA made from the mRNA. The sequence of the cloned cDNA can be determined using DNA sequencing techniques which are well known in the art. The cDNA can also be sequenced via the polymerase chain reaction (PCR).

Mismatches are hybridized nucleic acid duplexes which are not 100% complementary. The lack of total complementarity may be due to deletions, insertions, inversions, substitutions or frameshift mutations. Mismatch detection can be used to detect point mutations in a target nucleic acid. While these techniques can be less sensitive than sequencing, they are simpler to perform on a large number of tissue samples. An example of a mismatch cleavage technique is the RNase protection method, which is described in detail in Winter et al., Proc. Natl. Acad. Sci. USA, Vol. 82, p. 7575, 1985, and Meyers et al., Science, Vol. 230, p. 1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid mRNA or gene, but can a portion of the target nucleic acid, provided it encompasses the position suspected of being mutated. If the riboprobe comprises only a segment of the target nucleic acid mRNA or gene, it may be desirable to use a number of these probes to screen the whole target nucleic acid sequence for mismatches if desired.

In a similar manner, DNA probes can be used to detect mismatches, for example through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the target nucleic acid mRNA or DNA which might contain a mutation can be amplified before hybridization. Changes in target nucleic acid DNA can also be detected using Southern hybridization, especially if the changes are gross rearrangements, such as deletions and insertions,

Target nucleic acid DNA sequences which have been amplified may also be screened using allele-specific probes. These probes are nucleic acid oligomers, each of which contains a region of the target nucleic acid gene harboring a known mutation. For example, one oligomer may be about 30 nucleotides in length, corresponding to a portion of the target gene sequence. By use of a battery of such allele-specific probes, target nucleic acid amplification products can be screened to identify the presence of a previously identified mutation in the target gene. Hybridization of allele-specific probes with amplified target nucleic acid sequences can be performed, for example, on a nylon filter. Hybridization to a particular probe under stringent hybridization conditions indicates the presence of the same mutation in the tumor tissue as in the allele-specific probe.

Alteration of wild-type target genes can also be detected by screening for alteration of the corresponding wild-type protein. For example, monoclonal antibodies immunoreactive with a target gene product can be used to screen a tissue, for example an antibody that is known to bind to a particular mutated position of the gene product (protein). For example, an antibody that is used may be one that binds to a deleted exon (e.g., exon 14) or that binds to a conformational epitope comprising a deleted portion of the target protein. Lack of cognate antigen would indicate a mutation. Antibodies specific for products of mutant alleles could also be used to detect mutant gene product. Antibodies may be identified from phage display libraries. Such immunological assays can be done in any convenient format known in the art. These include Western blots, immunohistochemical assays and ELISA assays. Any means for detecting an altered protein can be used to detect alteration of wild-type target genes.

Primer pairs are useful for determination of the nucleotide sequence of a target nucleic acid using nucleic acid amplification techniques such as the polymerase chain reaction. The pairs of single stranded DNA primers can be annealed to sequences within or surrounding the target nucleic acid sequence in order to prime amplification of the target sequence. Allele-specific primers can also be used. Such primers anneal only to particular mutant target sequence, and thus will only amplify a product in the presence of the mutant target sequence as a template. In order to facilitate subsequent cloning of amplified sequences, primers may have restriction enzyme site sequences appended to their ends. Such enzymes and sites are well known in the art. The primers themselves can be synthesized using techniques which are well known in the art. Generally, the primers can be made using oligonucleotide synthesizing machines which are commercially available. Design of particular primers is well within the skill of the art.

Nucleic acid probes are useful for a number of purposes. They can be used in Southern hybridization to genomic DNA and in the RNase protection method for detecting point mutations already discussed above. The probes can be used to detect target nucleic acid amplification products. They may also be used to detect mismatches with the wild type gene or mRNA using other techniques. Mismatches can be detected using either enzymes (e.g., S1 nuclease), chemicals (e.g., hydroxylamine or osmium tetroxide and piperidine), or changes in electrophoretic mobility of mismatched hybrids as compared to totally matched hybrids. These techniques are known in the art. See Novack et al., Proc. Natl. Acad. Sci. USA, Vol. 83 p. 586, 1986. Generally, the probes are complementary to sequences outside of the kinase domain. An entire battery of nucleic acid probes may be used to compose a kit for detecting mutations in target nucleic acids. The kit allows for hybridization to a large region of a target sequence of interest. The probes may overlap with each other or be contiguous.

If a riboprobe is used to detect mismatches with mRNA, it is generally complementary to the mRNA of the target gene. The riboprobe thus is an antisense probe in that it does not code for the corresponding gene product because it is complementary to the sense strand. The riboprobe generally will be labeled with a radioactive, colorimetric, or fluorometric material, which can be accomplished by any means known in the art. If the riboprobe is used to detect mismatches with DNA it can be of either polarity, sense or anti-sense. Similarly, DNA probes also may be used to detect mismatches.

In some instances, the cancer does or does not overexpress c-met receptor and/or EGFR. Receptor overexpression may be determined in a diagnostic or prognostic assay by evaluating increased levels of the receptorprotein present on the surface of a cell (e.g. via an immunohistochemistry assay; IHC). Alternatively, or additionally, one may measure levels of receptor-encoding nucleic acid in the cell, e.g. via fluorescent in situ hybridization (FISH; see WO98/45479 published October, 1998), southern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

In some instances, the invention provides methods for reducing ErbB (e.g., EGFR) and/or c-met activity. Various methods for determining ErbB (e.g., EGFR) and/or c-met activity are known in the art and some are described and exemplified herein. Exemplary methods for measuring ErbB and/or c-met activity include, for example, examining on or more of the following: ErbB and/or c-met phorphorylation, ErbB and/or c-met kinase activity, and ErbB and/or c-met downstream signaling.

In some instances, the invention provides methods for reducing growth and/or proliferation of a cancer cell, or increasing apoptosis of a cancer cell. Methods for examining growth and/or proliferation of a cancer cell, or increasing apoptosis of a cancer cell are well known in the art and some are described and exemplified herein. Exemplary methods for determining cell grown and/or proliferation and/or apoptosis include, for example, BrdU incorporation assay, MTT, [³H]-thymidine incorporation (e.g., TopCount assay (PerkinElmer)), cell viability assays (e.g., CellTiter-Glo (Promega)), DNA fragmentation assays, caspase activation assays, tryptan blue exclusion, chromatin morphology assays and the like.
In some instances, the invention provides methods for restoring the sensitivity of a cancer cell to an ErbB antagonist (e.g., an EGFR antagonist), reducing resistance of a cancer cell to an ErbB antagonist (such as an EGFR antagonist), and/or treating acquired ErbB antagonist (such as an EGFR antagonist) resistance in a cancer cell. Methods for examining cell sensitivity and/or resistance to an ErbB antagonist (e.g., an EGFR antagonist) and/or resistance to an ErbB antagonist are known in the art and some are described herein. For example, the amount of cell growth and/or proliferation and/or amount of apoptosis may be determined, for example, in the presence of the ErbB antagonist. In other embodiments, the amount of cell growth and/or proliferation and/or amount of apoptosis may be determined in the presence of ErbB/c-met antagonist combination treatment as compared to the ErbB antagonist treatment alone.

In some instances, the invention provides methods for reducing PI3K (phosphoinositide-3 kinase) mediated signaling in a cancer cell. Methods for examining PI3K mediated signaling are known in the art and some methods are disclosed and exemplified herein. In some embodiments, the presence or absence of phosphorylated forms of proteins that are phosphorylated in response to PI3K activation (e.g., Akt) can be assayed using immunoassays.

### Chemotherapeutic Agents

The combination therapy of the invention can further comprise one or more chemotherapeutic agent(s). The combined administration includes coadministration or concurrent administration, using separate formulations or a single pharmaceutical formulation, and consecutive administration in either order, wherein preferably there is a time period while both (or all) active agents simultaneously exert their biological activities.

The chemotherapeutic agent, if administered, is usually administered at dosages known therefor, or optionally lowered due to combined action of the drugs or negative side effects attributable to administration of the antimetabolite chemotherapeutic agent. Preparation and dosing schedules for such chemotherapeutic agents may be used according to manufacturers' instructions or as determined empirically by the skilled practitioner.

Various chemotherapeutic agents that can be combined are disclosed above. Preferred chemotherapeutic agents to be combined are selected from the group consisting of a taxoid (including docetaxel and paclitaxel), vinca (such as vinorelbine or vinblastine), platinum compound (such as carboplatin or cisplatin), aromatase inhibitor (such as letrozole, anastrazole, or exemestane), anti-estrogen (e.g. fulvestrant or tamoxifen), etoposide, thiotepa, cyclophosphamide, methotrexate, liposomal doxorubicin, pegylated liposomal doxorubicin, capecitabine, gemcitabine, COX-2 inhibitor (for instance, celecoxib), or proteosome inhibitor (e.g. PS342).

### Formulations, Dosages and Administrations

The therapeutic agents used in the invention will be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, the drug-drug interaction of the agents to be combined, and other factors known to medical practitioners.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The therapeutic agents of the invention are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In the case of VEGF antagonists, local administration is particularly desired if extensive side effects or toxicity is associated with VEGF antagonism. An ex vivo strategy can also be used for therapeutic applications. Ex vivo strategies involve transfecting or transducing cells obtained from the subject with a polynucleotide encoding a c-met or EGFR antagonist. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

For example, if the c-met or EGFR antagonist is an antibody, the antibody is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Preferably the dosing is given by injections, most preferably intravenous or subcutaneous injections, depending in part on whether the administration is brief or chronic.

In another example, the c-met or EGFR antagonist compound is administered locally, e.g., by direct injections, when the disorder or location of the tumor permits, and the injections can be repeated periodically. The c-met or EGFR antagonist can also be delivered systemically to the subject or directly to the tumor cells, e.g., to a tumor or a tumor bed following surgical excision of the tumor, in order to prevent or reduce local recurrence or metastasis.

Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, the EGFR or c-met antagonist in the combination may be administered by intravenous injection while the protein kinase inhibitor in the combination may be administered orally. Alternatively, for example, both of the therapeutic agents may be administered orally, or both therapeutic agents may be administered by intravenous injection, depending on the specific therapeutic agents. The sequence in which the therapeutic agents are administered also varies depending on the specific agents.

Depending on the type and severity of the disease, about 1 μg/kg to 100 mg/kg (e.g., 0.1-20 mg/kg) of each therapeutic agent is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the cancer is treated, as measured by the methods described above. However, other dosage regimens may be useful. In one example, if the c-met or EGFR antagonist is an antibody, the antibody of the invention is administered every two to three weeks, at a dose ranging from about 5 mg/kg to about 15 mg/kg. If the c-met or EGFR antagonist is an oral small molecule compound, the drug may be administered daily at a dose ranging from about 25 mg/kg to about 50 mg/kg. Moreover, the oral compound of the invention can be administered either under a traditional high-dose intermittent regimen, or using lower and more frequent doses without scheduled breaks (referred to as “metronomic therapy”). When an intermittent regimen is used, for example, the drug can be given daily for two to three weeks followed by a one week break; or daily for four weeks followed by a two week break, depending on the daily dose and particular indication. The progress of the therapy of the invention is easily monitored by conventional techniques and assays.

The present application contemplates administration of the c-met and/or EGFR antagonist by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in-vivo delivery the nucleic acid is injected directly into the patient, usually at the site where the antibody is required. For ex vivo treatment, the patient's cells are removed, the nucleic acid is introduced into these isolated cells and the modified cells are administered to the patient either, directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

The following are examples of the methods and compositions of the invention. It is understood that various other embodiments may be practiced, given the general description provided above.

### EXAMPLES

### Example 1

### Analysis of C-met and EGFR Expression in NSCLC Cell Lines and Tumor Samples

### Materials and Methods

Microarray studies. Basal gene expression analysis of NSCLC cell lines and primary tumors was carried out using RNA extracted from sub-confluent cell cultures or frozen tumor lysates on the Affymetrix (Santa Clara, Calif.) microarray platform (HGU133Plus_2.0 chips). Preparation of complementary RNA, array hybridizations, and subsequent data analysis were carried out using manufacturer protocols, essentially as described in Hoffman E P et al., Nat Rev Genet 5:229-37 (2004).

To evaluate correlation of c-met expression with expression of other receptor tyrosine kinases (RTKs) expressed in NSCLC specimens, a variation filter was used to exclude genes with minimal variation across the samples being analyzed. Genes with minimal expression (those for which the absolute variation (max-min) across samples was <1000) were excluded from further analysis. In addition, a single probe was selected to represent a gene. Spearman rank correlation coefficients (ρ) were determined for each gene against MET mRNA (probe ID, 203510_at) or c-met protein (IHC).

Quantitative PCR. EGFR and MET mRNA expression levels were assessed by quantitative RT-PCR using standard Taqman techniques. Transcript levels were normalized to the housekeeping gene ribosomal protein L19 (RPL19) and results were expressed as either normalized expression values (=2^{-ΔCt}) or normalized expression relative to a pooled tissue source (=2^{-ΔΔCt}). The following primer/probe sets were utilized:

RPL19:
forward primer,
(SEQ ID NO: 26)
5′-ACCCCAATGAGACCAATGAAATC-3′,
reverse primer,
(SEQ ID NO: 27)
5′-ATCTTTGATGAGCTTCCGGATCT-3′,
probe,
(SEQ ID NO: 28)
5′(VIC)-AATGCCAACTCCCGTCAG-(MGBNFQ)-3′;
MET:
forward primer,
(SEQ ID NO: 29)
5′-CATTAAAGGAGACCTCACCATAGCTAAT-3′,
reverse primer,
(SEQ ID NO: 30)
5′-CCTGATCGAGAAACCACAACCT-3′,
probe,
(SEQ ID NO: 31)
5′(FAM)-CATGAAGCGACCCTCTGATGTCCCA-(BHQ-1)-3′,

Primer/probe sets for EGFR were purchased from Applied Biosystems (cat # 4331182, Hs00193306; Foster City, Calif.).

Immunohistochemistry (IHC). Formalin fixed and paraffin-embedded specimens were sectioned at 5 micron thickness onto slides. After deparaffinization and rehydration, sections were processed for c-Met and EGFR IHC analysis. EGFR IHC was performed with the EGFR pharmDx™ Kit (Dako, Glostrup, Denmark) according to the Manufacturer's instructions. For c-met immunohistochemistry (IHC), antigen retrieval was performed using preheated Target Retrieval buffer (Dako, Glostrup, Denmark) at 99° C. for 40 minutes for the c-met IHC. Endogenous peroxidase activity was quenched with KPL Blocking Solution (KPL, Gaithersburg, Md.) at room temperature for 4 minutes. Endogenous avidin/biotin was blocked with Vector Avidin Biotin Blocking Kit (Vector Laboratories, Burlingame, Calif.). Subsequently, sections were incubated with 10 μg/ml mouse anti-c-met (clone DL-21) monoclonal antibody (Upstate Biotechnology Inc. Lake Placid, N.Y.) in blocking serum for 60 minutes at room temperature, and followed by incubation with biotinylated secondary horse anti-mouse antibody for 30 minutes. Vectastain ABC Elite Reagent (Vector Laboratory, Burlingame, Calif.) with Metal Enhanced DAB (Pierce Biotechnology, Inc. Rockford, Ill.) was used to develop the slides. The levels of expression were defined as negative (−), weak (+), moderate (++) or strong (+++). Cell lines or tumor specimen that contain more than 10% tumor cells with weak, moderate, or strong staining were considered positive.

Cell Culture and tumor samples. Cell lines were obtained from the American Type Culture Collection, the NCI Division of Cancer Treatment and Diagnosis, and the Japanese Health Sciences Resources depositories as shown in Table 1. All cell lines were maintained in RPMI 1640 supplemented with 10% FBS (Sigma, St. Louis, Mo.), and 2 mM L-glutamine. Tumor samples were obtained from University of Michigan, Cybrdio, Cooperative Human Tissue Network and Integrated Laboratory services.

TABLE 1
Cell lines used in Examples.

| cell line | Source | Taqman | IHC |
|---|---|---|---|
| A427 | ATCC* | X | |
| A549 | ATCC | X | X |
| ABC-1 | Japan | | X |
| | Health Sci** | | |
| Calu-1 | ATCC | X | X |
| EBC-1 | Japan | | X |
| | Health Sci | | |
| EKVX | NCI- | X | X |
| | DCTD*** | | |
| H1155 | ATCC | X | |
| H1299 | ATCC | X | X |
| H1435 | ATCC | X | X |
| H1568 | ATCC | X | X |
| H1650 | ATCC | X | X |
| H1651 | ATCC | X | X |
| H1666 | ATCC | X | X |
| H1703 | ATCC | X | X |
| H1781 | ATCC | X | X |
| H1793 | ATCC | X | X |
| H1838 | ATCC | X | X |
| H1975 | ATCC | X | X |
| H2009 | ATCC | X | X |
| H2030 | ATCC | X | X |
| H2122 | ATCC | X | X |
| H2126 | ATCC | X | X |
| H226 | ATCC | X | X |
| H23 | ATCC | X | X |
| H2405 | ATCC | X | X |
| H292 | ATCC | X | X |
| H322T | ATCC | X | X |
| H358 | ATCC | X | X |
| H441 | ATCC | X | X |
| H460 | ATCC | X | X |
| H520 | ATCC | X | X |
| H522 | ATCC | X | X |
| H596 | ATCC | X | X |
| H647 | ATCC | X | X |
| H650 | ATCC | X | X |
| H661 | ATCC | X | X |
| H838 | ATCC | X | X |
| HLFα | ATCC | X | |
| HOP 18 | NCI- | X | X |
| | DCTD | | |
| HOP 62 | NC- | X | X |
| | DCTD | | |
| HOP 92 | NCI- | X | X |
| | DCTD | | |
| KNS-62 | Japan | | X |
| | Health Sci | | |
| LXFL | NCI- | X | X |
| 529 | DCTD | | |
| RERF- | Japan | | X |
| LC-Ad1 | Health Sci | | |
| RERF- | Japan | | X |
| LC-KJ | Health Sci | | |
| RERF- | Japan | | X |
| LC-MS | Health Sci | | |
| RERF- | Japan | | X |
| LC-OK | Health Sci | | |
| SK- | ATCC | X | X |
| MES-1 | | | |
| SW1573 | ATCC | X | X |
| VMRC- | Japan | | X |
| LCD | Health Sci | | |

*American Culture Type Collection
**Japanese Health Sciences Resources
***National Cancer Institute Division of Cancer Treatment and Diagnosis

### Results

MET mRNA Expression Correlates with EGFR mRNA Expression in NSCLC Cell Lines.

To evaluate whether the expression of c-met is correlated with the expression of EGFR and other receptor tyrosine kinases (RTKs) in NSCLC cell lines, spearman rank correlation coefficients were determined from microarray-based gene expression data generated from the 50 NSCLC cell lines shown in Table 1. EGFR and MET mRNA levels were positively correlated in cell lines (ρ=0.54, p<0.0001) and EGFR expression was highly correlated with MET expression (Table 2).

TABLE 2
Correlation of RTK mRNA expression with
MET mRNA expression in NSCLC cell lines.

| | Spearman | p-value |
|---|---|---|
| Gene | ρ | (two-tailed) |
| EPHA2 | 0.5516 | P < 0.0001 |
| EGFR | 0.5412 | P < 0.0001 |
| EPHB2 | 0.5169 | 0.0001 |
| ROR1 | 0.5115 | 0.0001 |
| MST1R | 0.4719 | 0.0005 |
| EPHA1 | 0.4219 | 0.0023 |
| EPHA4 | 0.4217 | 0.0023 |
| ERBB3 | 0.3736 | 0.0075 |
| DDR1 | 0.2985 | 0.0352 |
| EPHB4 | 0.2751 | 0.0532 |
| ERBB2 | 0.2533 | 0.0759 |
| AXL | 0.2396 | 0.0938 |
| STYK1 | 0.1389 | 0.336 |
| EPHB6 | 0.1219 | 0.3989 |
| KIT | 0.08365 | 0.5636 |
| PDGFRB | 0.0557 | 0.7008 |
| TEK | −0.009277 | 0.949 |
| PDGFRA | −0.03757 | 0.7956 |
| EPHA3 | −0.04528 | 0.7548 |
| TYRO3 | −0.05786 | 0.6898 |
| MERTK | −0.07213 | 0.6187 |
| INSR | −0.1031 | 0.476 |
| FGFR4 | −0.1037 | 0.4737 |
| RYK | −0.1587 | 0.271 |
| FGFR2 | −0.1653 | 0.2513 |
| PTK7 | −0.1683 | 0.2427 |
| EPHA5 | −0.1693 | 0.2399 |
| EPHA7 | −0.1712 | 0.2346 |
| IGF1R | −0.1782 | 0.2157 |
| DDR2 | −0.2249 | 0.1164 |
| FGFR3 | −0.2382 | 0.0957 |
| FGFR1 | −0.4131 | 0.0029 |

cMET Protein Expression Correlates with EGFR mRNA Expression in NSCLC Cell Lines

To evaluate whether c-met protein expression, determined by immunohistochemistry (IHC), is correlated with expression of EGFR and other receptor tyrosine kinases (RTKs) in NSCLC cell lines, spearman rank correlation coefficients were determined from microarray-based gene expression data generated in the 50 NSCLC cell lines shown in Table 1. EGFR mRNA and c-met protein levels were positively correlated in the cell lines (ρ=0.50, p=0.002) and EGFR expression was highly correlated with expression of o-met protein (Table 3).

TABLE 3
Correlation of RTK mRNA expression with c-MET
protein expression (IHC) in NSCLC cell lines.

| | Spearman | p-value |
|---|---|---|
| Parameter | ρ | (two-tailed) |
| MET | 0.789 | P < 0.0001 |
| EPHB2 | 0.5651 | P < 0.0001 |
| EPHA2 | 0.5154 | 0.0002 |
| EGFR | 0.5005 | 0.0002 |
| ROR1 | 0.4653 | 0.0008 |
| MST1R | 0.4386 | 0.0016 |
| EPHA1 | 0.4316 | 0.002 |
| ERBB2 | 0.3246 | 0.0229 |
| AXL | 0.3165 | 0.0267 |
| EPHA4 | 0.2748 | 0.0561 |
| ERBB3 | 0.2628 | 0.0681 |
| EPHB4 | 0.2362 | 0.1023 |
| DDR1 | 0.2354 | 0.1034 |
| STYK1 | 0.1163 | 0.4263 |
| TYRO3 | 0.09579 | 0.5126 |
| KIT | 0.04308 | 0.7688 |
| PDGFRB | 0.04063 | 0.7816 |
| IGF1R | −0.000919 | 0.995 |
| EPHB6 | −0.002974 | 0.9838 |
| MERTK | −0.02735 | 0.852 |
| FGFR2 | −0.06236 | 0.6703 |
| TEK | −0.07868 | 0.591 |
| PDGFRA | −0.1085 | 0.4579 |
| PTK7 | −0.1471 | 0.3132 |
| EPHA3 | −0.1693 | 0.245 |
| DDR2 | −0.1699 | 0.2432 |
| RYK | −0.1741 | 0.2316 |
| FGFR4 | −0.1801 | 0.2155 |
| INSR | −0.1891 | 0.1932 |
| EPHA5 | −0.2246 | 0.1208 |
| EPHA7 | −0.2925 | 0.0414 |
| FGFR3 | −0.3264 | 0.0221 |
| FGFR1 | −0.5078 | 0.0002 |

C-MET mRNA Expression Correlates with EGFR mRNA Expression in NSCLC Tumor Samples.

To evaluate whether c-met mRNA expression is correlated with expression of EGFR and other receptor tyrosine kinases (RTKs) in the NSCLC cell lines shown in Table 1, spearman rank correlation coefficients were determined from microarray-based gene expression data generated from 78 NSCLC tumors. Expression of EGFR mRNA and C-MET mRNA was positively correlated in NSCLC tumors (ρ=0.26, p=0.02) (Table 4).

TABLE 4
Correlation of RTK mRNA expression with
MET mRNA expression in NSCLC tumors.

| | Spearman | p-value |
|---|---|---|
| Parameter | ρ | (two-tailed) |
| MST1R | 0.5856 | P < 0.0001 |
| EPHA2 | 0.4247 | 0.0001 |
| CSF1R | 0.3249 | 0.0037 |
| EPHA1 | 0.3104 | 0.0057 |
| ERBB2 | 0.2952 | 0.0087 |
| AXL | 0.2912 | 0.0097 |
| EPHB2 | 0.2572 | 0.023 |
| EGFR | 0.2564 | 0.0235 |
| KDR | 0.1973 | 0.0833 |
| DDR2 | 0.1856 | 0.1037 |
| PDGFRB | 0.1827 | 0.1094 |
| EPHB4 | 0.1763 | 0.1227 |
| ERBB3 | 0.1749 | 0.1257 |
| TEK | 0.1514 | 0.1858 |
| EPHA4 | 0.1311 | 0.2526 |
| DDR1 | 0.07695 | 0.5031 |
| ALK | 0.03423 | 0.7661 |
| INSR | −0.07637 | 0.5063 |
| PTK7 | −0.07702 | 0.5027 |
| MERTK | −0.0794 | 0.4895 |
| EPHA3 | −0.1008 | 0.38 |
| PDGFRA | −0.1296 | 0.2581 |
| FGFR1 | −0.142 | 0.2149 |
| FGFR2 | −0.1688 | 0.1397 |
| FGFR3 | −0.1812 | 0.1123 |
| EPHB3 | −0.2269 | 0.0457 |
| IGF1R | −0.2673 | 0.018 |
| EPHB1 | −0.3318 | 0.003 |
| KIT | −0.3878 | 0.0005 |
| RYK | −0.3959 | 0.0003 |
| EPHA7 | −0.5231 | P < 0.0001 |

### Coexpression of EGFR and C-MET in NSCLC Cell Lines and Primary Tumors.

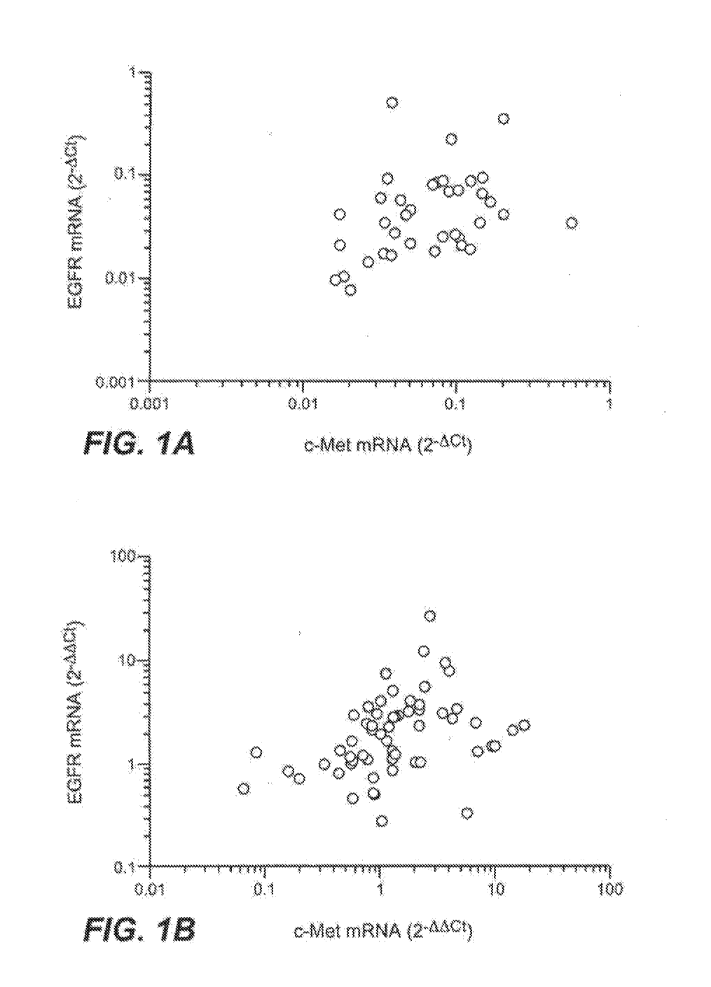

To evaluate whether c-met and EGFR are coexpressed in NSCLC cell lines and primary tumor samples, expression of EGFR and C-MET mRNA was determined by quantitative RT-PCR in a panel of NSCLC cell lines (as indicated in Table 1) or frozen primary NSCLC tumor lysates. EGFR and C-MET mRNA levels were positively correlated in cell lines (ρ=0.59, p<0.0001) (FIG. 1, left panel) and primary NSCLC specimens (ρ=0.48, p=0.0003) (FIG. 1, right panel). These data demonstrate that there is an overlap in the expression of C-MET and EGFR in NSCLC cell lines and primary tumor samples.

### Confirmation of EGFR and C-MET Coexpression by IHC NSCLC Cell Lines and Primary Tumors.

Forty-seven non-small cell lung cancer (NSCLC) cell lines (as indicated in Table 1) and one hundred thirty eight primary NSCLC samples (Genentech collection) were examined for their c-met and EGFR IHC expression by IHC. The levels of expression were scored as negative (−), weak (+), moderate (++) or strong (+++), and a cell line or tumor specimen that contained more than 10% tumor cells with weak, moderate, or strong staining was scored as positive.

79% (37/47) of cell lines and 68% (94/138) of NSCLC tumors stained positive for EGFR (Table 5). The EGFR positive samples (79% of cell lines and 68% of primary tumors) were further stratified based on their c-met expression levels (Table 5). The EGFR positive cell lines exhibited weak (22%), moderate (57%) and strong (19%) c-met expression, and the EGFR-positive primary tumor samples were only weakly or moderately positive. The adenocarcinoma subtype were more commonly positive for c-met staining than the squamous cell subtype (70% versus 40%), with more cases of moderate staining (30% versus 7%). These data demonstrate a significant overlap between c-met and EGFR expression in NSCLC cell lines and tumor samples, particularly in the adenocarcinoma tumor subtype.

TABLE 5
EGFR and C-MET protein coexpression in
NSCLC cell lines and primary tumors.

| Tissue | Histopathological | c-Met IHC score in EGFR+ specimens | | | |
|---|---|---|---|---|---|
| Source | Subtype | − | + | ++ | +++ |
| Cell lines* | | 3% | 22% | 57% | 19% |
| | | (n = 1) | (n = 8) | (n = 21) | (n = 7) |
| Tumors** | Adenocarcinoma | 30% | 40% | 30% | 0% |
| | | (n = 14) | (n = 19) | (n = 14) | |
| | Squamous cell | 61% | 33% | 7% | 0% |
| | | (n = 28) | (n = 15) | (n = 3) | |

*79% (37/47) NSCLC cell lines stained positive for EGFR
**68% (94/138) NSCLC tumors stained positive for EGFR

### Example 2

### Reduction of C-met Protein Expression in NSCLC Cells Increases Ligand-Induced Activation of EGFR, Her2 and Her3

Materials and Methods

Retroviral shRNA constructs. Oligonucleotides coding shRNA sequences against c-met (5′-GATCCCCGAACAGAATCACTGACATATTCAAGAGATATGTCAGTGATTCTGTTCTTTT TTGGAAA-3′ (SEQ ID NO: 32) (shMet 3) and

5′GATCCCCGAAACTGTATGCTGGATGATTCAAGAGATCATCCAGCATACAGTT TCTTTTTTGGAAA (SEQ ID NO: 33) (shMet 4)) were cloned into BglII/HindIII sites of the pShuttle-H1 vector downstream of the H1 promoter (David Davis, GNE). BOLD text signifies the target hybridizing sequence. These constructs were recombined with the retroviral pHUSH-GW vector (Gray D et al BMC Biotechnology. 2007; 7:61) using Clonase II enzyme (Invitrogen), generating a construct in which shRNA expression is tinder control of an inducible promoter. Treatment with the tetracycline analog doxycycline results in shRNA expression. The shGFP2 control retroviral construct containing a shRNA directed against GFP (Hoeflich et al. Cancer Res. (2006) 66(2):999-1006) was provided by David Davis, Genentech, Inc. shGFP2 contains the following oligonucleotide:

(EGFP) shRNA

(sense) 5′-GATCCCCAGATCCGCCACAACATCGATTCAAGAGATCGATGTTGTGGCGGATCTTGTTTT TTGGAAA-3 (SEQ ID NO:34).

Cell Culture. GP-293 packaging cells (Clontech) were maintained in HGDMEM (GNE) supplemented with 10% Tet-Free FBS (Clontech), 2 mM L-Glutamine (GNE), and 100 U/ml penicillin and 100 U/ml streptomycin (Gibco). H441 cells (ATCC No. HTB-174) were maintained in 50:50 media (DMEM:F12, MediaTech) supplemented with 10% Tet-Free FBS (Clontech), 2 mM L-Glutamine (GNE), and 100 U/ml penicillin and 100 U/ml streptomycin (Gibco). EBC-1 cells (Japanese Health Sciences Resources; see Cancer Res. (2005) 65(16):7276-82) were maintained in RPMI 1640 (GNE) supplemented with 10% Tet-Free FBS (Clontech), 2 mM L-Glutamine (GNE), and 100 U/ml penicillin and 100 U/ml streptomycin (Gibco). Cells were maintained at 37° C. with 5% CO2.

Development of recombinant retrovirus and stable lines. GP-293 packaging cells were cotransfected using FuGene 6 (Roche) and CalPhos Mammalian Transfection kit (Clontech) with pVSV-G (Clontech) and the above recombinant retroviral constructs. Media containing the recombinant virus was then added to EBC-1 and H441 cells and cells were selected in Puromycin (Clontech). Cells stably expressing retroviral constructs were then autocloned via FACS into 96 well plates.

Western blot. To resolve proteins, 20 ug of whole cell lysate was run on 4-12% Bis-Tris NuPAGE gel with MOPS buffer (Invitrogen). Gels were equilibrated in 2× NUPAGE transfer buffer with anti-oxidant buffer then transferred to 0.2 um PVDF membrane by iBlot. Membranes were blocked in TBST (10 mM TRIS, pH 8.0, 150 mM NaCl, 0.1% Tween 20) containing 5% BSA for one hour at room temperature then incubated overnight in primary antibody diluted in blocking buffer at 4° C. Membranes were washed with TBST then incubated with the HRP-conjugated secondary antibody (GE Healthcare) in TBST with 5% nonfat milk for one hour at room temperature. Antibodies were detected by chemiluminescence (GE Healthcare, ECL Plus).

Screening of stable cell lines. Clones stably transduced with retroviral constructs were grown in the appropriate media +/−1 μug/ml doxycycline (Clontech) to induce expression of the shRNA, and screened via western blots for c-met knockdown using anti-c-met C-12 antibody (Santa Cruz Biotech). Phospho-c-met was blotted for using anti-Phospho-c-met Y1003 (Biosource) and anti-Phospho-c-met Y1234/1234 (Cell Signaling) antibodies. As a control, actin was blotted for using anti-Actin I-19 antibody (Santa Cruz Biotech). EBC Clone 3.15 and EBC clone 4.12 showed strong reduction of met expression and phospho c-met levels, H441 Clone 3.11 and H441 Clone 3.1 showed intermediate reduction of c-met expression and phospho-met expression, and EBC clone 4.5 showed a smaller reduction of c-met and phospho-c-met expression.

Cell lines EBC clone 4.5, EBC clone 4.12 contained construct shMet4 and cell lines H441 Clone 3.1, H441 Clone 3.11, and EBC Clone 3.15 contained construct shMet 3.

Ligand response experiments. Cells passaged with/without doxycyline for 48 hours (EBC shMet) or 6 days (H441 shMet) were plated at 1×10⁶ cells/well in a 6-well dish with/without Dox (0.1 ug/ml) in 10% FBS-RPMI then incubated overnight at 37 C. Cells were rinsed with PBS, and media was changed to 0.5% BSA-RPMI (with/without doxycyline) to serum starve cells for 2 hours at 37 C. Media containing ligand (20 nM TGFa or 2 nM HRG) was added to wells and incubated for 20 minutes at 37 C. Wells were rinsed with cold TBS then lysed with TBS, 1% NP-40, Complete protease inhibitor cocktail (Roche) and phosphatase inhibitor cocktails 1 and 2 (Sigma). The monolayer and supernatant was scraped from the well and transferred to microfuge tubes where the lysate was incubated on ice for 10-30 minutes. Cell debris was pelleted by microfuge, and the supernatent was transferred to a fresh tube. Protein concentration was quantified by BCA assay (Pierce), and lysates were stored at −20 C until thawed for electrophoresis. 20 ug (EBC1) or 15 ug (H441) of whole cell lysate were run on gels and blotted for phospho-c-met (YY1234/35, 3126 from Cell Signaling Technology), total c-met (C12, sc-10 from San Cruz Biotechnology), b-actin (I-19, sc-1616 from Santa Cruz Biotechnology), phospho-EGFR (Y1173, 04-341 from Upstate), total EGFR (MI-12-1, from MBL), phospho-Her2 (YY1121/22, 2243 from Cell Signaling Technology), total Her3 (C18, sc-284, from San Cruz Biotechnology), phospho-Her3 (Y1289, 4791, from Cell Signaling Technology), or total Her3 (C17, sc-285, from Santa Cruz Biotechnology) as described above.

### Results

Retroviruses carrying tetracycline-inducible short-hairpin RNA (shRNA) that target c-met were used to generate stable NSCLC cell line clones that could be induced to express shRNAs to knockdown c-met expression. To examine the effect of c-met knockdown on expression and phosphorylation of EGFR family members in NSCLC cell line EBC1, EBC1 shMet 4.12 cells containing an inducible shRNA directed against met or control shRNA directed against GFP were grown in control media or media containing 0.1 ug/ml Dox for 48 hours. After serum-starvation for two hours, cells were untreated or treated with TGFα or Heregulin b1 for 20 minutes. Whole cell lysates were evaluated for expression of total and phospho-proteins as indicated.

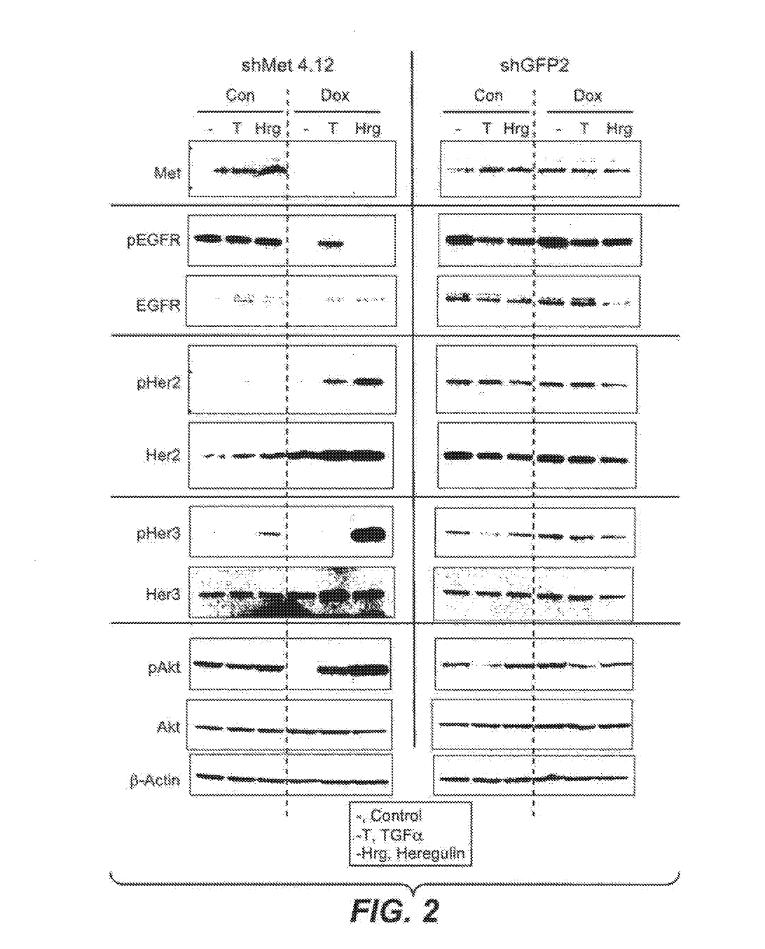

Dox-treated EBC1 cells in which c-met protein expression was knocked-down using shRNA (FIG. 2; EBCshMet 4.12, Dox, left panel), but not Dox-treated control EBC1 cells (FIG. 2; shGFP2, right panel) showed increased pEGFR and pHer2 in response to TGFa treatment and increased pHer3 in response to Heregulin treatment, as well as increased pAKT with either TGFa or Heregulin treatment. The Dox-treated EBC shMet 4.12 cells (no ligand stimulation) showed increased total Her2 and total Her3, and decreased pEGFR and pHer3. EBC1 cells did not show robust induction of pEGFR, pHer2, pHer3, or pAKT in response to TGFa or Heregulin treatment in the absence of c-met knock-down.

To examine the effect of c-met knockdown on expression and phosphorylation of EGFR family members in another NSCLC cell line, NSCLC H441 cells containing an inducible shRNA directed against met or control shRNA directed against GFP were grown in control media or media containing 0.1 ug/ml Dox for 48 hours. After serum-starvation for 2 hours, cells were untreated or treated with TGFα or Heregulin b1 for 20 minutes. Whole cell lysates were evaluated for expression of total and phospho-proteins as indicated.

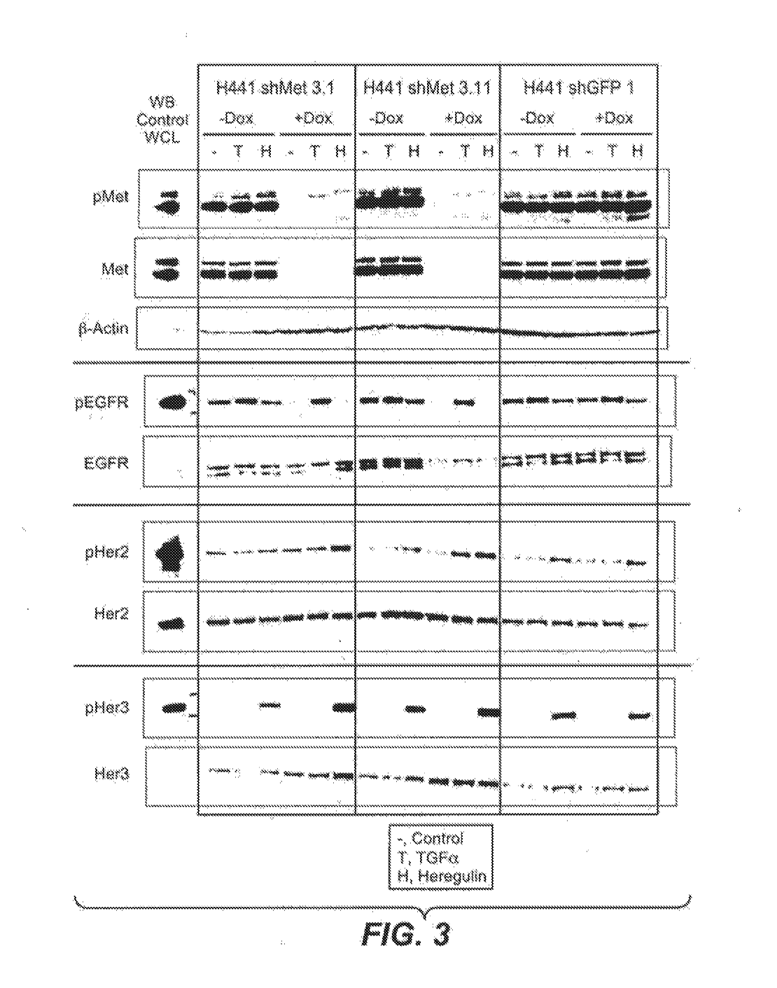

H441 cells in which c-met was knocked-down using shRNA (FIG. 3; Dox-treated shMet 3.1, left panel and Dox-treated shMet 3.11 middle panel), but not Dox-treated control H441 cells (FIG. 3; shGFP1, right panel) showed enhanced pHer2 and pHer3 in response to Heregulin treatment. The Dox treated shMet 3.1 and shMet 3.11 cells also show increased total Her3 and decreased pEGFR. Unlike EBC1 cells, H441 cells have a slight response to TGFα (pEGFR) and Heregulin (pHer2 and pHer3) without c-met knock-down. EBC1 cells have higher c-met levels than H441 cells.

These experiments demonstrated that reduction of c-met expression in NSCLC cell lines leads to decreased basal activation of EGFR (pEGFR) and increased ligand-induced activation of Her 2 and Her3, suggesting that c-met inhibition increases sensitivity to ligands of the EGF family.

### Example 3

### The Combination of C-met Knockdown and Treatment with EGFR Inhibitor Erlotinib Significantly Inhibited Tumor Growth in a Xenograft Model.

To test whether EGFR plays a role in maintaining tumor survival in cell in which c-met function is partially inhibited, EBC-1 shMet-4.5 tumor bearing animals were treated with combinations of erlotinib (Tarceva™) and Dox.

### Materials and Methods

Test material. Erlotinib (Tarceva™) was provided by OSI Pharmaceuticals to the Formulations Department at Genentech and was weighed out along with a sufficient amount of vehicle (methylcellulose tween (MCT)). Materials were stored in a refrigerator set to maintain a temperature range of 4° C. to 8° C. Anti-c-met monovalent monoclonal antibody MetMAb (rhuOA5D5v2) (WO2007/063816) was provided by the Antibody Engineering Department at Genentech, Inc., in a clear liquid form. The EBC-1 cell line was obtained from Japanese Collection of Research Bioresources (JCRB).

Species. Forty nude mice (nu/nu) were obtained from Charles River Laboratories (CRL) and were acclimatized for at least one week prior to being put on study. Animals were housed in ventilated caging systems in rooms with filters supplying High-Efficiency Particulate Air (HEPA). Only animals that appeared to be healthy and were free of obvious abnormalities were used for the study.

Study design. EBC-1 cells were cultured in growth media that consisted of RPMI 1640 media (Invitrogen), 2 mM L-glutamine, and 10% fetal bovine serum. To prepare cells for inoculation into mice, cells were trypsinized, washed with ten milliliters of sterile 1× phosphate buffered saline (PBS). A subset of cells was counted by trypan blue exclusion and the remainder of cells was resuspended in 100 μl of sterile 1× PBS to a concentration of 5×10⁷ cells per milliliter. Mice were inoculated subcutaneously in the right sub-scapular region with 5×10⁶ EBC-1 cells. Tumors were monitored until they reached a mean volume of 300 mm³.

Mice implanted with tumor cells were randomized into four groups of ten mice each treatment was initiated (summarized in Table 6). Mice in Group 1 (control group) were treated with 100 μL vehicle control, methylcellulose tween (MCT), every day (QD) via oral gavage (PO) and were switched to drinking water containing 5% sucrose. Mice in Group 2 (c-met knockdown group) were treated with 100 μL MCT, QD, PO, but were switched to drinking water containing 0.5 mg/mL of doxycycline (Dox) in 5% sucrose. Mice in Group 3 (erlotinib treated group) were treated with 100 mg/kg of erlotinib in a volume of 100 μL formulated in MCT, QD, PO and were switched to drinking water containing 5% sucrose. Mice in Group 4 (c-met knockdown plus erlotinib treated group) were treated with 100 mg/kg of erlotinib in a volume of 100 μL formulated in MCT, QD, PO and were switched to drinking water containing 1 mg/mL of doxycycline (Dox) in 5% sucrose. Dox and sucrose water was changed every 2-3 days. Erlotinib and MCT were dosed for 14 days, stopped for 6 days and then resumed for the remainder of the study (20 days). Animals were taken off study if tumors reached greater than 1000 mm³ or tumors showed signs of necrotic lesions. If more than 3 animals had to be taken off study from any given group, treatment in that group was halted and all animals were taken off study. All studies and handling of mice complied with the Institutional Animal Care and Use Committee (IACUC) guidelines.

TABLE 6
Study Design

| | | | | | | | Dose |
|---|---|---|---|---|---|---|---|
| | | | | | Dose. | Dose Conc. | Volume |
| Group | No./Sex | Test Material | route | Dose Frequency | (mg/kg) | (mg/ml) | (μl) |
| 1 | 10/F | MCT, | PO; | Every day (QD) | 0 | 0 | 100 |
| | | 5% sucrose | drinking | for 2 weeks, halted | | | |
| | | water | water | for 6 days and | | | |
| | | | | then restarted until | | | |
| | | | | end of study; via | | | |
| | | | | drinking water | | | |
| 2 | 10/F | MCT, | PO; | Every day (QD) | 0.5 mg/mL | 0.5 (Dox)* | 100 |
| | | 1 mg/mL Dox | drinking | for 2 weeks, halted | in | | |
| | | in 5% sucrose | water | for 6 days and | drinking | | |
| | | water | | then restarted until | water | | |
| | | | | end of study; via | | | |
| | | | | drinking water | | | |
| 3 | 10/F | Erlotinib, | PO; | Every day (QD) | 100 | 25 (erlotinib) | 100 |
| | | 5% sucrose | drinking | for 2 weeks, halted | | | |
| | | water | water | for 6 days and | | | |
| | | | | then restarted until | | | |
| | | | | end of study; via | | | |
| | | | | drinking water | | | |
| 4 | 10/F | Erlotinib, | PO; | Every day (QD) | 100; 1 mg/mL | 25 | 100 |
| | | 1 mg/mL Dox | drinking | for 2 weeks, halted | in | (erlotinib); 1 | |
| | | in 5% sucrose | water | for 6 days and | drinking | (Dox) | |
| | | water | | then restarted until | water | | |
| | | | | end of study; via | | | |
| | | | | drinking water | | | |

*in US patent application No. 61/034,446, Dox dosage was incorrectly stated to be 1 mg/ml. The correct dose is 0.5 mg/ml, as indicated above.

Tumor and Body Weight Measurement. Tumor volumes were measure in two dimensions (length and width) using UltraCal-IV calipers (Model 54-10-111, Fred V. Fowler Company, Inc.; Newton, Mass.). The following formula was used with Excel v11.2 (Microsoft Corporation; Redmond, Wash.) to calculate tumor volume:

Tumor Volume (mm³)=(length·width²)·0.5

Efficacy Data Analysis. Tumor inhibition was plotted using KaleidaGraph 3.6 (Synergy Software; Reading, Pa.). Percent growth inhibition (% Inh) at Day 17 was calculated as follows:

% Ihn=100× [Tumor Size (Vehicle)−{Tumor Size (MetMAb)/Tumor Size (Vehicle)}]

Tumor incidence (TI) was determined by the number of measurable tumors in each group at Day 17. Partial regression (PR) is defined as tumor regression of >50% but <100% of starting tumor volume at any day during the study. Complete regression (CR) is defined as tumor regression of 100% from initial starting tumor volume at any day during the study.

Mean tumor volume and standard error of the mean (SEM) were calculated using JMP software, version 5.1.2 (SAS Institute; Cary, N.C.). Data analysis and generation of p-values using either Student's t-test or the Dunnett's t-test was also done using JMP software, version 5.1.2.

### Results

The Combination of c-met Knockdown and Erlotinib Treatment Significantly Inhibited Tumor Growth in a Xenograft Model.

To investigate the role of c-met in driving tumor growth in the EBC-1 model, stable EBC-1 clones that could be induced to express shRNAs to knockdown c-met expression were generated using retroviruses carrying a tetracycline-inducible short-hairpin RNA (shRNA) targeting c-met. The EBC-1 non-small cell lung cancer (NSCLC) cell line is highly amplified for c-met and expresses high amounts of the c-met receptor which acts in a ligand-independent manner to drive cell and tumor growth. The EGFR gene is wildtype in the EBC-1 cell line.

Following induction of shRNA expression with the tetracycline analog doxycycline, clone EBC-1 shMet-3.15 showed efficient, largely complete knock-down of c-met expression. Induction of shRNA also blocked proliferation of these cells, as analyzed in Cell Titer Glo or Alamar Blue cell viability assays. Growth arrest followed by apoptosis was observed in EBC1 shMet3.15 cells 24-72 hours after shRNA induction. The same cell line clone was implanted into an animal model essentially as described above (except that animals were not treated with erlotinib) and permitted to form tumors. Induction of shRNA expression after tumor formation in these animals resulted in tumor regression in vivo. These results demonstrated that c-met expression is essential for the growth and survival of EBC-1 cells in vitro and in vivo.

The EBC-1 shMet-4.5 clone displayed partial knocked-down of c-met expression following induction of shRNA expression with Dox. Reduction in c-met expression also resulted in effects upon cell growth and survival in this clone: induction of shRNA expression decreased cell number when assayed in in vitro cell viability assays, and induction of shRNA expression after tumor formation in a xenograft model inhibited tumor growth but did not cause tumor regression.

Clone shMet-4.5 was selected for use in experiments evaluating the effect of combining knock-down of c-met expression with erlotinib treatment, as described below.

The EBC-1 shMet-4.5 NSCLC cell line was inoculated into nude mice and then animals were monitored for tumor growth until the engrafted cells had formed tumors of about 300 mm³. Mice were then grouped into four treatment arms; Group 1: Vehicles, Group 2: Doxycycline (Dox), Group 3: erlotinib (100 mg/kg), and Group 4: erlotinib+Dox (See Table 6).

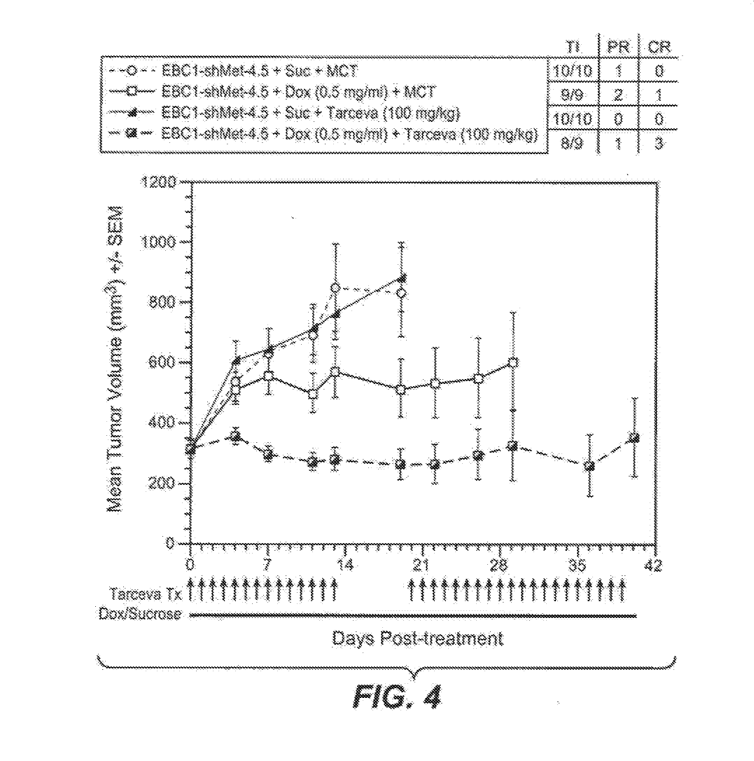

Treatment of mice with erlotinib had no effect upon tumor growth (−6% tumor inhibition; FIG. 4), whereas treatment with Dox (inhibiting met expression) resulted in 38% reduction in tumor growth compared to the vehicle control at day 19 (FIG. 4; Student's t-test, p=0.084), falling just shy of statistical significance. However, the reduction of tumor growth was statistically significant when compared with the erlotinib only group (Student's t-test, p=0.004; FIG. 4). Combination of erlotinib and Dox resulted in a dramatic improvement in efficacy, resulting in a 68% reduction in tumor growth compared to vehicle control at day 19 (Student's West, p=0.001; FIG. 4). Treatment with the combination of erlotinib and Dox also resulted in statistically significant reduction in tumor growth when compared with treatment with Dox alone (Student's t-test, p=0.03) or treatment with erlotinib alone (Student's t-test, p<0.0001).

Treatment with Dox and erlotinib resulted in a higher number of partial responses (PR; defined as tumor regression of >50% but <100% of starting tumor volume at any day during the study) and complete responses (CR; defined as tumor regression of 100% front initial starting tumor volume at any day during the study). Specifically, combination of erlotinib plus Dox resulted in 1 PR and 3 CRs, whereas treatment with erlotinib resulted in no PRs or CRs and treatment with Dox (c-Met knockdown) resulted in 2 PRs and 1 CR. These data demonstrate that the combination of met inhibition (Dox treatment) and EGFR inhibition (erlotinib treatment) is more likely to induce complete tumor regressions than inhibition of c-met or EGFR alone, even though analysis of the individual animal tumor data revealed that not all tumors responded strongly to the combination of c-met inhibition and erlotinib.

These results show that inhibition of c-met and EGFR in the EBC-1 shMet-4.5 xenograft model resulted in a significant reduction in tumor growth. Thus, tumors in which c-met expression and activity are partially inhibited utilize the EGFR pathway to ensure tumor growth and survival. This indicates that EGFR plays a role in tumor survival and growth in tumors in which c-met is inhibited.

### Example 4

### Treatment with an Anti-C-met Antibody and the EGFR Inhibitor Erlotinib Showed a Dramatic Improvement in Efficacy Verses Treatment with Anti-C-met Antibody or Erlotinib Alone

### Materials and Methods

Test Material. Anti-c-met monovalent monoclonal antibody MetMAb (rhuOA5D5v2) was provided by the Antibody Engineering Department at Genentech, Inc., in a clear liquid form at 10.6 mg/ml. The vehicle was 10 mM histidine succinate, 4% trehalose dihydrate, 0.02% polysorbate 20, pH 5.7. Erlotinib (TARCEVA™) was provided by OSI Pharmaceuticals to the Pharmaceutics Department at Genentech and was weighed out along with a sufficient amount of vehicle (methylcellulose tween (MCT)). All material was shipped from Genentech, Inc. to the Van Andel Research Institute (VARI; Grand Rapids, Mich.) and was formulated prior to animal treatments. Materials were stored in a refrigerator set to maintain a temperature range of 4° C. to 8° C. The NCI-H596 cell line was obtained from American Type Culture collection (Manassas, Va.).

Species. Forty human HGF transgenic C3H-SCID mice (hu-HGF-Tg-C3H-SCID) were obtained from the in-house colony at the Van Andel Research Institute (VARI; Grand Rapids, Mich.). Five C3H-SCID mice were obtained from Jackson Laboratories. Animals were 4-6 weeks old and weighed 21-22 grams each. Mice were acclimated to study conditions for at least three days prior to tumor cell inoculations. Mice were housed in a shower-in barrier facility. Animals were housed in ventilated caging systems in rooms with filters supplying High-Efficiency Particulate Air (HEPA). Only animals that appeared to be healthy and were free of obvious abnormalities were used for the study.

Study design. As most HGF responsive tumors are driven in a paracrine fashion, a xenograft model that models paracrine driven growth was selected. Mouse HGF is a poor ligand for human c-met leading to a low biological response of human c-met expressing cells lines to mouse HGF (Bhargava, M., et al., 1992; Rong, S., et al., 1992). Therefore, to model paracrine HGF-driven human tumors, transgenic mice (hu-HGF-Tg-SCID) that express human HGF in a ubiquitous fashion from the metallotheionein promoter were generated (Zhang, Y., et al., 2005). Serum HGF levels in the hu-HGF-Tg-SCID mice are ˜5-10-fold higher than physiological levels (1-5 ng/mL vs. 0.2-0.5 ng/mL) and cells lines that respond to HGF by proliferating in vitro show a potent enhancement of tumor growth when grown as xenograft tumors in hu-HGF-Tg-SCID mice.

The NCI-H596 non-small cell lung cancer (NSCLC) cell line was selected as for in vivo efficacy studies in hu-HGF-Tg-SCID mice because the cell line is highly HGF responsive and an anti-c-met antibody, MetMAb, blocks HGF-driven proliferation of this cell line in vitro (Kong-Beltran, M., et al., 2006). The NCI-H596 cell line bears a mutated form of the c-met gene lacking exon 14 that encodes a binding site for the E3 ubiquitin ligase Cbl (Kong-Beltran, M., 2006). The Cbl-binding site is phosphorylated at tyrosine 1003 (Y1003) following HGF binding, allowing for Cbl to bind and ubiquitinate c-met, thus targeting it for proteosomal degradation (Peschard, P., et al., 2001). The responsiveness of NCI-H596 can also be seen in vivo, as the cell line readily form tumors in HGF-Tg-SCID mice (expressing human HGF, as noted above), but will not form tumors in immunocompromised mice lacking human HGF (nu/nu nude mice or SCID mice). NCI-H596 cells are considered to form c-met-driven tumors. NCI-H596 cells possess a wild-type EGFR gene and are sensitive to EGFR inhibitor erlotinib (TARCEVA™) when grown in the presence of TGFα, as demonstrated by reduced cell viability when grown in the presence of erlotinib and TGFα.

NCI-H596 cells were cultured in growth media that consisted of RPMI 1640 media (Invitrogen), 2 mM L-glutamine, and 10% fetal bovine serum. To prepare cells for inoculation into mice, cells were trypsinized, washed with ten milliliters of sterile 1× phosphate buffered saline (PBS). A subset of cells was counted by trypan blue exclusion and the remainder of the cells was resuspended in 100 μl of sterile 1× PBS to a concentration of 5×10⁶ cells per milliliter.

Mice were prepared for inoculation by shaving the dorsal area with clippers. The following day each mouse was inoculated subcutaneously in the right sub-scapular region with 5×10⁵ NCI-H596 cells. Tumors were monitored until they reached a mean volume of 100 mm³.

HGF-Tg-C3H-SCID mice were randomized into two groups of eleven mice each and given an intraperitoneal injection of test material twice weekly for four weeks. Animals in Group 1 were given 100 μl of vehicle and animals in Group 2 were given 30 mg/kg of the anti-c-met monovalent monoclonal antibody MetMAb. The study design is presented in Table 7. Tumors were measured three times per week for five weeks, starting on the day of treatment. Mice were euthanized after five weeks, although some animals were euthanized earlier due to large tumor volumes (>1500 mm3). Control C3H-SCID mice were also inoculated to serve as a negative control for tumor growth and were monitored for tumor growth for five weeks.

All studies and handling of mice complied with the Institutional Animal Care and Use Committee (IACUC) guidelines.

TABLE 7
Study Design

| | | | | | | | Dose |
|---|---|---|---|---|---|---|---|
| | | | | | Dose. | Dose Conc. | Volume |
| Group | No./Sex | Test Material | route | Dose Frequency | (mg/kg) | (mg/ml) | (μl) |
| 1 | 10/F | Vehicles: | PO; IP | Every day (QD) | 0 | 0 | 100 (ea.) |
| | | Captisol; | | for 2 weeks; Once | | | |
| | | MetMAb | | | | | |
| | | buffer | | | | | |
| 2 | 10/F | Erlotinib | PO | Every day (QD) | 150 | 30 | 100 |
| | | | | for 2 weeks | | | |
| 3 | 10/F | MetMAb | IP | Once | 30 | 6 | 100 |
| 4 | 10/F | Erlotinib + | PO; IP | Every day (QD) | 150; 30 | 30; 6 | 100 (ea.) |
| | | MetMAb | | for 2 weeks; Once | | | |

Tumor and Body Weight Measurement. Tumor volumes were measure in two dimensions (length and width) using UltraCal-IV calipers (Model 54-10-111, Fred V. Fowler Company, Inc.; Newton, Mass.). The following formula was used with Excel v11.2 (Microsoft Corporation; Redmond, Wash.) to calculate tumor volume:

Tumor Volume (mm³)=(length·width²)·0.5

Efficacy Data Analysis. Tumor inhibition was plotted using KaleidaGraph 3.6 (Synergy Software; Reading, Pa.). Percent growth inhibition (% Inh) at Day 17 was calculated as follows:

% Ihn=100× [Tumor Size (Vehicle)−{Tumor Size (MetMAb)/Tumor Size (Vehicle)}]

Tumor incidence (TI) was determined by the number of measurable tumors in each group at Day 17. Partial regression (PR) is defined as tumor regression of >50% but <100% of starting tumor volume at any day during the study. Complete regression (CR) is defined as tumor regression of 100% from initial starting tumor volume at any day during the study.

Mean tumor volume and standard error of the mean (SEM) were calculated using JMP software, version 5.1.2 (SAS Institute; Cary, N.C.). Data analysis and generation of p-values using either Student's t-test or the Dunnett's t-test were performed using JMP software, version 5.1.2.

Kaplan-Meier survival curve estimates were drawn for time to tumor doubling for each group. Pairwise comparisons between groups were made. Statistical comparisons were made with the log-rank test. Data analysis was performed using JMP software.

### Results

The NCI-H596 NSCLC cell line was inoculated into hu-HGF-Tg-C3H-SCID animals and animals were monitored for tumor growth until the engrafted cells had formed tumors of about 100 mm³. Mice were then grouped into four treatment arms; group 1: Vehicle, group 2: Erlotinib, group 3: MetMAb, and group 4: Erlotinib+MetMAb (See Table 7). Groups treated with MetMAb were dosed only once whereas groups treated with erlotinib were dosed every day for two weeks and then treatment was stopped and tumor growth was monitored two to three times per week. C3H-SCID control mice were also inoculated and monitored for growth of NCI-H596 tumors not exposed to human HGF.

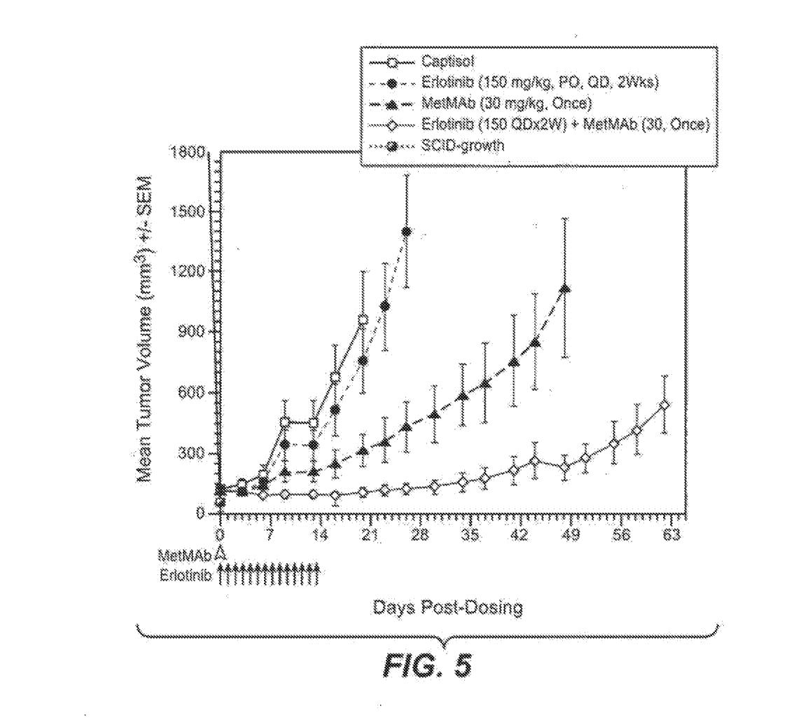

Growth of NCI-H596 tumors was vastly improved in the context of the hu-HGF-Tg-C3H-SCID mice compared to the C3H-SCID control mice (FIG. 5; compare vehicle control group to C3H-SCID). Treatment of mice with anti-e-met monovalent monoclonal antibody MetMAb resulted in a 67% reduction in tumor growth compared to the vehicle control at day 20 (FIG. 5; Student's t-test, p=0.0044), consistent with previous studies of MetMAb in the NCI-H596 models. Treatment of NCI-H596 tumor-bearing mice with erlotinib resulted in a statistically insignificant reduction in tumor growth compared to the vehicle control at day 20 (FIG. 5; Student's t-test, p=0.165). Treatment with the combination of MetMAb and erlotinib showed a dramatic improvement in efficacy, resulting in an 89% reduction in tumor growth compared to vehicle control at day 20 (FIG. 5; Student's t-test, p=0.0035).

Treatment of mice with MetMAb resulted in a 67% reduction in tumor growth compared to the vehicle control at day 20 (FIG. 5; Student's t-test, p=0.0044), consistent with previous studies of MetMAb in the NCI-H596 models. Treatment of NCI-H596 tumor-bearing mice with erlotinib resulted in a statistically insignificant reduction in tumor growth compared to the vehicle control at day 20 (FIG. 5; Student's t-test, p=0.165). Treatment with the combination of MetMAb and erlotinib showed a dramatic improvement in efficacy over either agent alone, resulting in an 89% reduction in tumor growth compared to vehicle control at day 20 (FIG. 5; Student's t-tests; MetMAb+erlotinib vs. vehicle, day 20—p=0.0035; MetMAb+erlotinib vs. erlotinib alone, day 26—p=0.0009; MetMAb+erlotinib vs. MetMAb alone, day 48 p=0.0149).

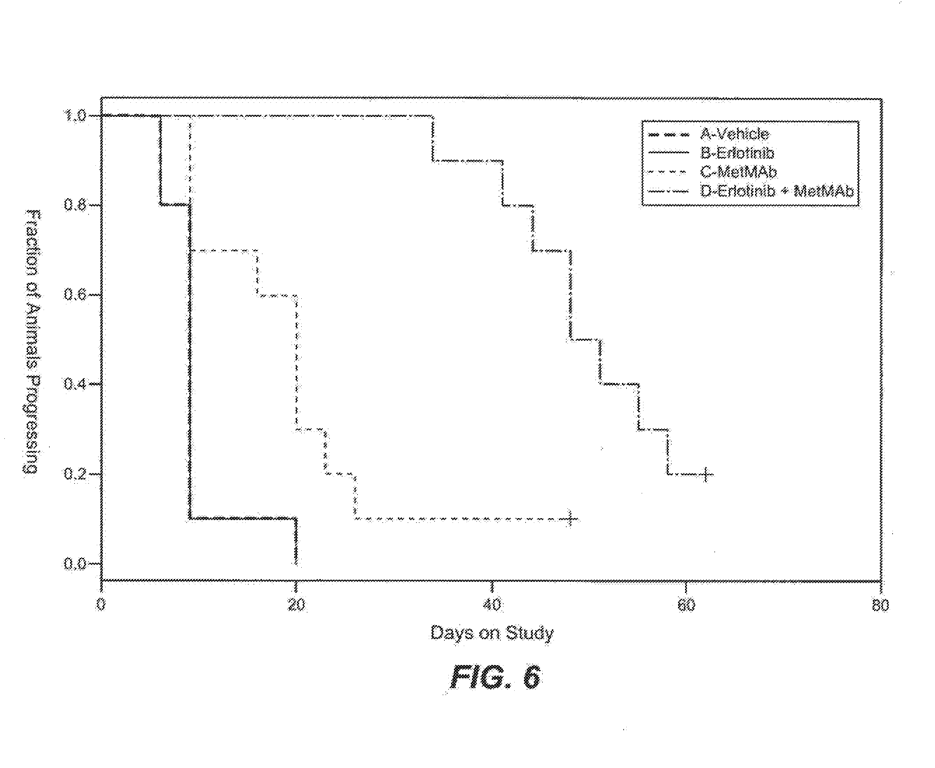

Tumor volume data were collected for nine weeks after the dosing ended to address whether the combination of MetMAb plus erlotinib resulted in improvements in time to tumor progression. To address this issue, time to tumor doubling (TTD) measurements, defined as the time it took for tumors to double in size, were calculated for each group and used to generate Kaplan-Meier survival curves. The combination of MetMAb plus erlotinib showed a dramatic improvement in time to tumor progression with a mean TTD of 49.5 (±2.6) days versus 17.8 (±2.2) days for the MetMAb-treated group, 9.5 (±1.2) days for the erlotinib-treated group, and 9.5 (±1.2) days for vehicle control group (FIG. 6). These data show that the combination of MetMAb plus erlotinib significantly improves the time to tumor progression versus either single agent alone (Log-rank test; vehicle vs. MetMAb—p<0.0001; vehicle vs. MetMAb+erlotinib—p<0.0001; erlotinib vs. MetMAb+erlotinib p<0.0001 and MetMAb vs. MetMAb+erlotinib—p=0.0009).

These data demonstrate that treatment with the combination of MetMAb and erlotinib results in highly significant improvements in tumor growth inhibition and tumor progression relative to treatment with MetMAb or erlotinib alone.

### Example 5

### C-met Signaling Regulates EGFR Signaling

### Materials and Methods

Microarray analyses: Three microarray experiments were performed using Affymetrix HGU133 Plus 2.0 arrays. In each case, preparation of complementary RNA, array hybridizations, and subsequent data analysis were carried out using manufacturer protocols, essentially as described in Hoffman E P et al., Nat Rev Genet 5:229-37 (2004). Raw expression data, in the form of Affymetrix CEL files, were normalized as a group to remove non-biological sources of variation between data for individual samples using the RMA method of normalization (Irizarry, Biostatistics, 2003, PubMed ID 12925520) as implemented in the Partek GS 6.3b software package (Partek, Saint Louis, Mo.). The resulting normalized, log 2 scale expression values were analyzed as follows and were transformed to the linear scale for plotting purposes.

In the first experiment, ligand-responsive NSCLC HOP92 and H596 cells were untreated or stimulated with 50 ng/ml HGF for 6 hrs before mRNA expression profiling. Briefly, cells were plated in 6-well plates at approximately 5×10⁵ cells/well. After a day, cells were washed, then transferred to RPMI media+0.1% BSA. On day 3, cells were stimulated for 6 h with HGF at 50 ng/ml in RPMI medium+0.1% BSA. Cells were washed once with cold PBS, lysed with RNAeasy lysis buffer, and RNA prepared according to the manufacturer's protocol. HOP92 and H596 samples were analyzed separately using a t-test to measure the significance (P-value) of the difference in expression levels for each gene in the +HGF and −HGF conditions. These P-values were converted to Q-values by correcting for multiple testing using the Benjamini and Hochberg method (Benjamini and Hochberg, 1995). Genes were then ranked on statistical significance (Q-value) of the expression level difference in each cell line.

In the second experiment, mRNA expression levels of clones EBCshMet3-15 and EBCshMet4-12 were assayed after 24 and 48 hrs of incubation with or without 50 ng/ml doxycycline. The expression pattern of each Affymetrix probe set (gene) was analyzed using a linear statistical model (ANOVA) that estimated the effect of clone (3-15 or 4-12), treatment (control or doxycycline), and time-point (24 or 48 hours) as well as the interaction of time-point and treatment effects. The ANOVA procedure produced measures of significance (P-values) for each of these four effects. These P-values were converted to Q-values by correcting for multiple testing using the Benjamini and Hochberg method. Genes were then ranked on statistical significance (Q-value) of the expression level difference between doxycycline and control samples.

In the third experiment, EBCMet shRNA 4-12 cell or control EBCGFP shRNA cells were incubated in media alone or media with 50 ng/ml doxycycline for 24 h. After further treatment (+/− HGF 100 ng/ml for 2 hours), mRNA expression was assayed by microarray. The expression pattern of each Affymetrix probe set (gene) was analyzed using a linear statistical model (ANOVA) that estimated the effect of the shRNA target (Met or GFP), shRNA induction (doxycycline or control), and HGF treatment as well as the interaction of these three variables. These P-values were then converted to Q-values of the expression level difference between plus-HGF and minus-HGF conditions in doxycycline-treated EBCMetshRNA4-12 samples. Using cutoff of a Q-value of 0.05 (5% False Discovery Rate) and a two-fold expression change for the comparison of the +/− HGF groups, 188 probesets were selected.

TGFα ELISA: EBC-1-shMet xenograft tumors were generated and Dox was dosed essentially as described in Example 3, except that Dox was used at 1 mg/ml in 5% sucrose and tumors were allowed to grow to 300-400 mm³ prior to initiation of treatment. Animals were dosed for 3 days, then sacrificed. Flash frozen EBC-1-shMet-4.12 xenograft tumor samples were placed into 2 mls of cold lysis buffer (PBS+1% TritonX-100+Phosphatase Cocktail 2 (Sigma cat# P5726)) and Complete Mini EDTA-Free protease inhibitor (Roche #11 836 170 001)(1 tablet per 10 mls of solution). Tumors were homogenized with a hand held homogenizer and lysates were incubated on ice for 1 hr with occasional swirling. Lysates were spun down at 10000×G for 10 minutes at 4° C., transferred to a new tube and Her 3 protein was quantified using a BCA assay (Pierce cat# 23225).

Anti-TGF-alpha polyclonal antibody (R&D Systems, Minneapolis, Minn.) was diluted to 1 μg/ml in phosphate buffered saline (PBS) and coated onto ELISA plates (25 μL/well plates with MaxiSorp surface, Nunc, Neptune, N.J.) during an overnight incubation at 4° C. After washing 6 times with wash buffer (PBS/0.05% Tween-20), the plates were blocked with PBS/0.5% bovine serum albumin (BSA) for 1 to 2 hr. This and all subsequent incubations were performed at room temperature on an orbital shaker. Samples were diluted using sample buffer (PBS/0.5% BSA/0.5% Tween-20/0.2% bovine gamma globulin/0.25% CHAPS/5 mM EDTA/10 ppm Proclin). Using the same buffer, serial dilutions were prepared of recombinant human TGF-alpha (R&D Systems), with a standard curve range of 400-12.5 pg/ml. Frozen control samples pre-diluted to quantitate at the high, mid, and low regions of the standard curve were thawed. Plates were washed six times, and the samples, standards, and controls were added (25 μL/well) and incubated for 2 hr. After washing the plates twelve times, biotinylated goat anti-TGFalpha polyclonal antibody (R&D Systems) diluted to 1 μg/ml in sample buffer was added (25 μL/well). Following a one hour incubation, the plates were washed twelve times. Streptavidin-horse radish peroxidase (GE Healthcare, Piscataway, N.J.) diluted 1/4,000 in sample buffer was then added (25 μL/well). After a final 30 min incubation, the plates were washed twelve times, and tetramethyl benzidine (TMB, Kirkegaard & Perry Laboratories, Gaithersburg, Md.) was added. Color was allowed to develop for 6 to 8 minutes at room temperature, and the reaction was stopped by the addition of 1 M phosphoric acid. Absorbance values were obtained using a microplate reader (450 nm, 620 reference), and the sample concentrations were calculated from 4-parameter fits of the standard curves.

### Results

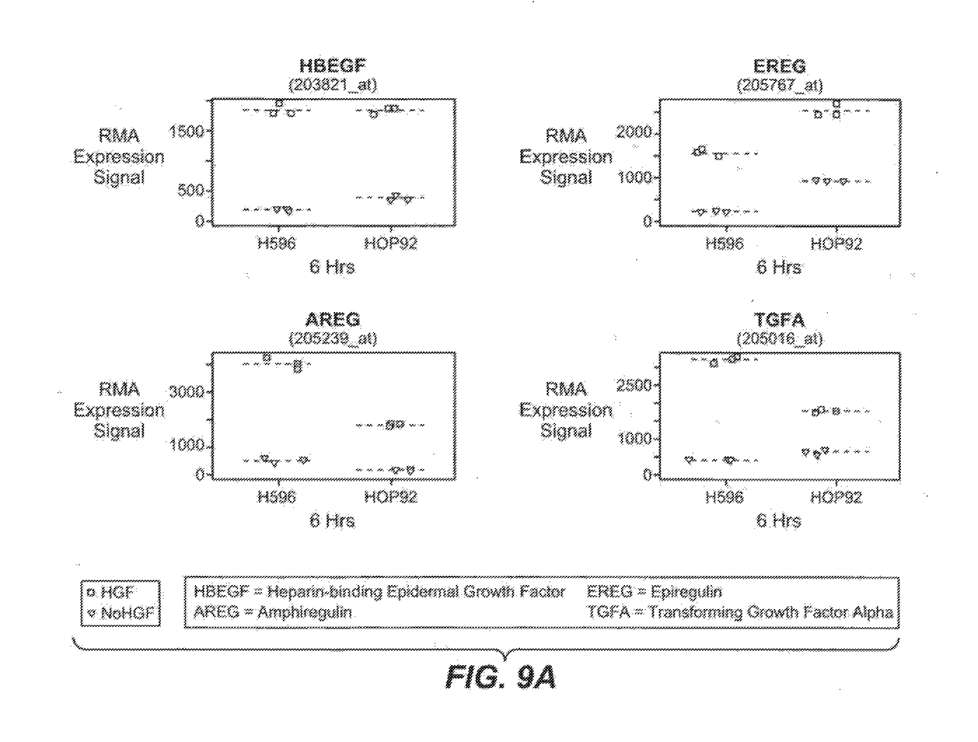

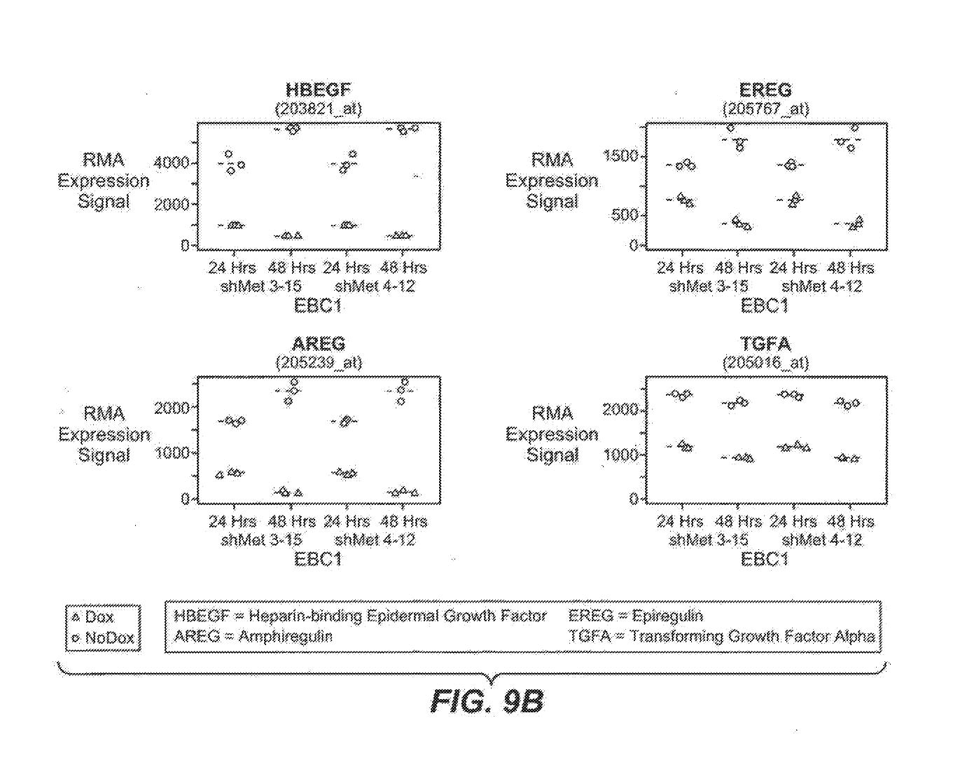

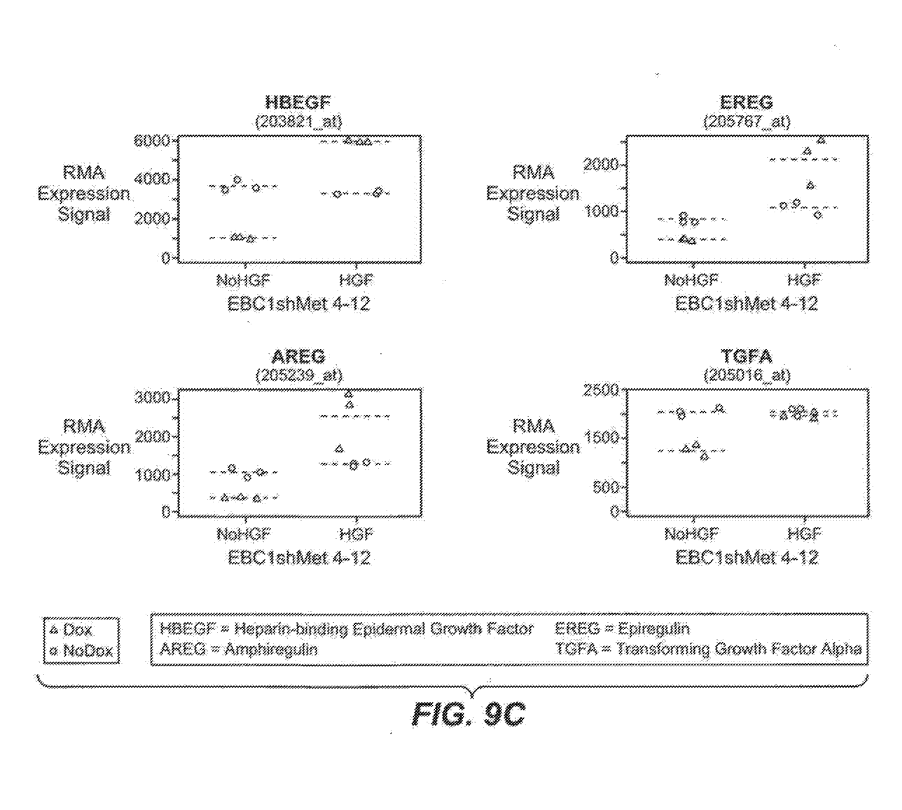

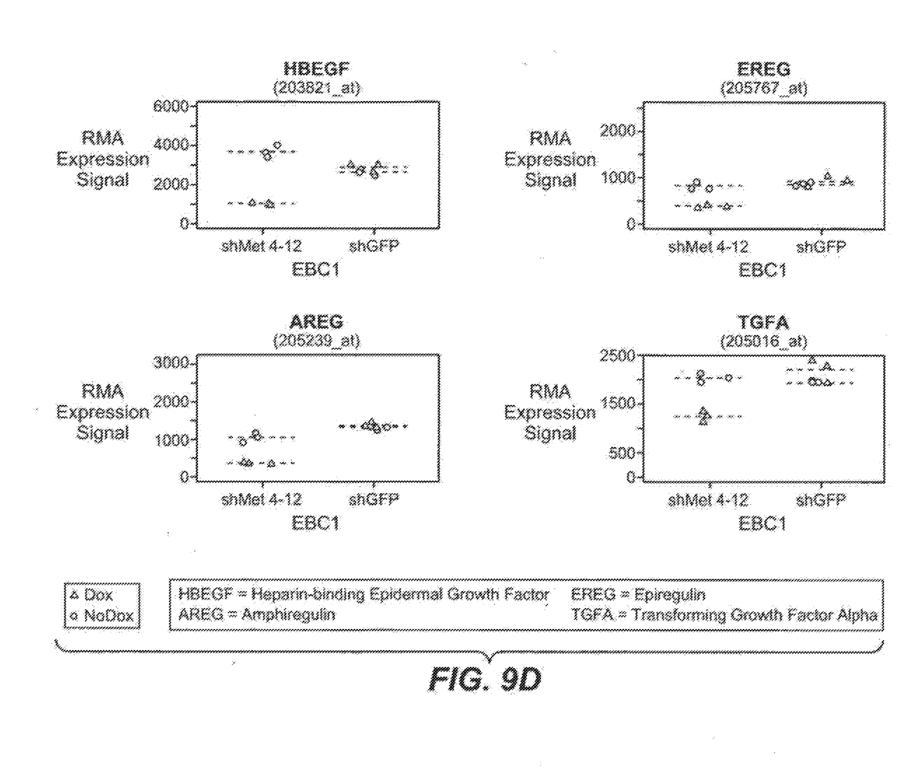

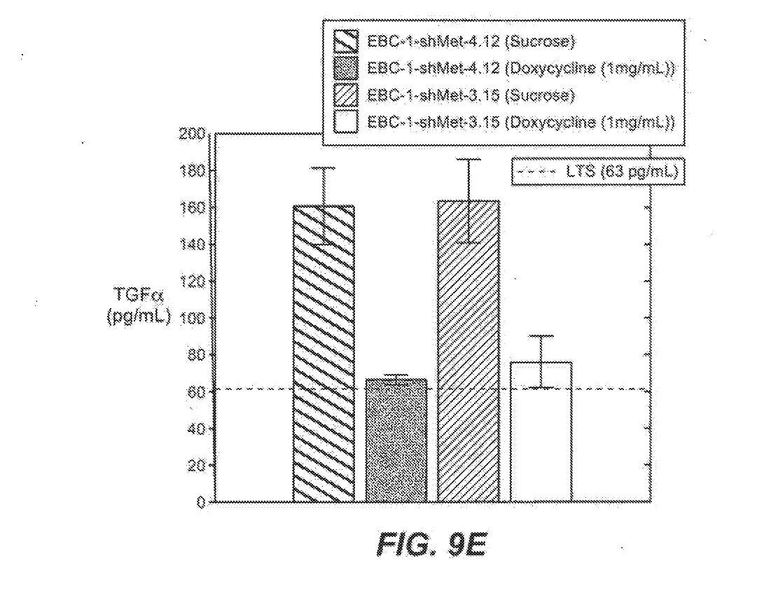

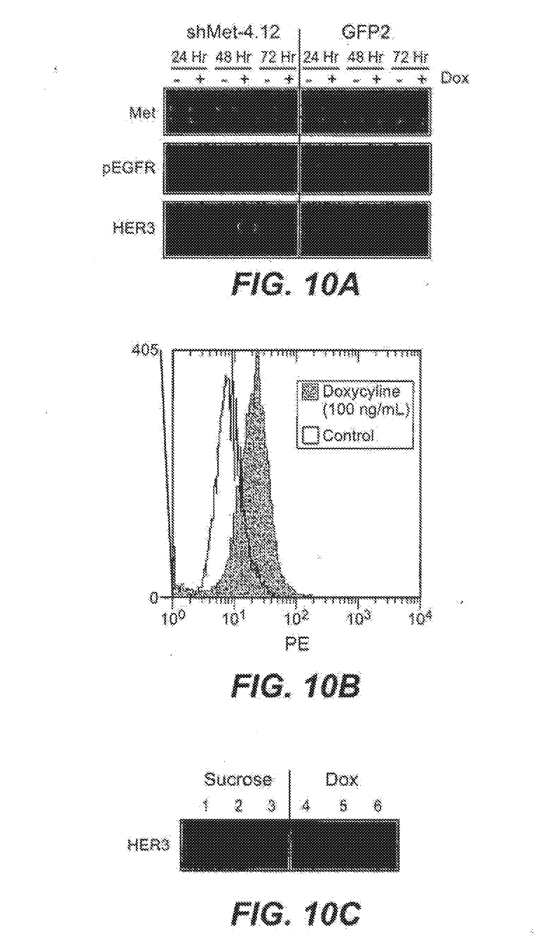

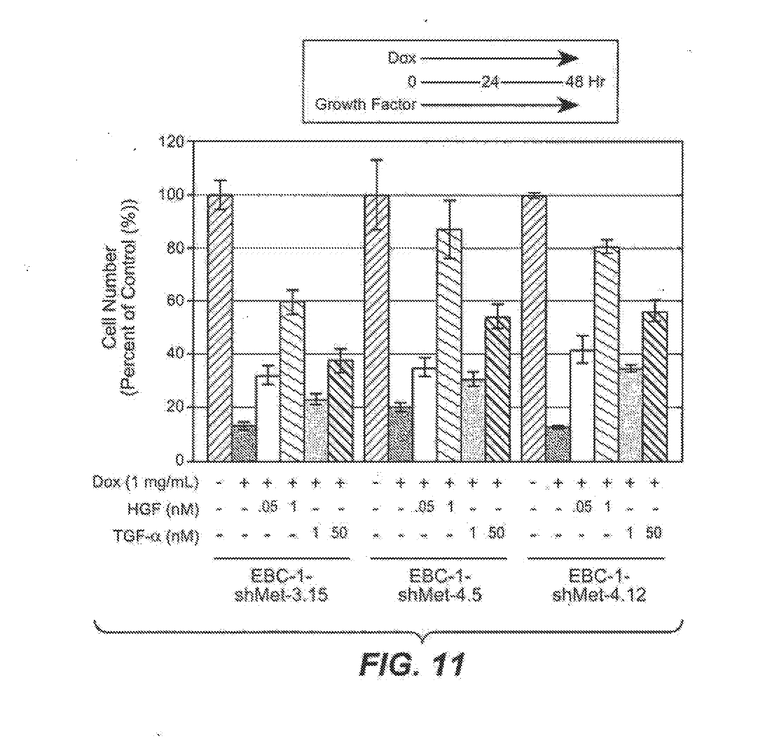

Activation of c-met by HGF treatment increased mRNA expression of EGFR ligands (HB-EGF, Epiregulin, Amphiregulin, TGFα) in ligand-responsive NSCLC cell lines Hop-92 and NCI-H596 (FIG. 9A). Conversely, inhibition of c-met expression using shRNA in ligand-independent NSCLC cell line EBC1 cells reduced mRNA expression of those EGFR ligands (FIG. 9B). HGF treatment of dox-treated EBC1shMet cell line 4-12 restored expression of EGFR ligands (FIG. 9C). Reduction of EGFR ligands did not occur in control EBC-1 cells that expressed a siRNA directed against GFP (FIG. 9D). Reduction of c-met expression in EBC-1-shMet xenograft tumors resulted in a decrease in tumor TGFα protein levels at day 3 post-treatment (FIG. 9E).
These data demonstrate that c-met activity can regulate EGFR signaling in c-met amplified HGF-independent cells (EBC1) as well as HGF-dependent cell lines (Hop92 and NCI-H596). More specifically, c-met signaling increased and maintained expression of EGFR family of ligands, which could then stimulate their own EGFR family of receptors in an autocrine manner. Conversely, inhibition of c-met signaling resulted in decreased expression of EGFR ligands. Interference with this autocrine loop is a likely cause of the decreased pEGFR observed in EBC1 cells following c-met knockdown (FIG. 10) and the increased sensitivity to ligand-induced activation of EGFR following c-met knockdown described in Example 2. These results suggest that EGFR activity can compensate for loss of c-met signaling activity in HGF-dependent and HGF-independent tumors, and are consistent with the dramatically increased xenograft tumor efficacy observed when tumors were treated with the combination of EGFR and c-met inhibitors (Example 4).

### Example 6

### C-met Activity Regulates HER3 Expression

### Materials and Methods

Western blot analysis of pEGFR and Her3 protein: Cells were plated at a density of 1×10⁶ and incubated 18 hours at 37 C in 10% Tet-approved FBS in RPMI 1640. The next day, media was removed and replaced with fresh normal media, with or without 0.1 ug/ml Dox. 24, 48 and 72 hours after changing media, proteins were extracted with 1% NP-40/TBS/Roche's Complete protease inhibitor cocktail/Sigma's phosphatase inhibitor cocktails 1 and 2 after a cold TBS rinse. 15 ug of total protein was loaded on Invitrogen's 4-12% Bis-Tris NUPADE gel with MOPS buffer and transferred to PVDF by Invitrogen's iBlot. Membranes were immunoblotted for phosphorylated proteins (pEGFR (Y1173) Upstate 04-341 at a dilution of 1:1000 in 5% BSA/TBST), stripped with Pierce's Restore stripping buffer, then reprobed for total proteins (c-met: SCBT sc-10 at 1:10,000 dilution; Her3: SCBT sc-285 at 1:2000 dilution in 5% nonfat dry milk and TBST). Proteins were detected with Amersham's HRP-conjugated secondary antibodies (Amersham anti-rabbit-HRP, #NA934V; Amersham anti-mouse-HRP) using Amersham's ECL Plus chemiluminescent kit according to the manufacturer's instructions.

Her3 FACS: EBC-1 shMet 4-12 cells were seeded at 10⁶ cells per 10 cm plate in RPMI 1640 (as above) and plates were incubated overnight. Dox was added to plates to a final concentration of 100 ng/ml. Plates were incubated for 48 hours. Following incubation, cells were trypsinized, centrifuged, then resuspended in cold 200 μL PBS+2% FBS (FACS Buffer) and transferred to 96 well plates. Cells were spun down and resuspended in FACS buffer plus 10 μg/ml of Her3:1638 (3E9.2G6) antibody from Genentech. Cells were incubated for 1 hour on ice, then washed with cold FACS Buffer and resuspended in FACS buffer+1:200 RPE conjugated F(ab′)₂ Goat anti-mouse IgG+IgM (H+L) (Jackson Immuno cat# 115-116-068). Cells were incubated on ice for 30 minutes, then washed once with cold FACS buffer and resuspended in FACS buffer plus 7AAD (BD Pharmingen cat#559925). FACS analysis was performed according to the manufacturer's instructions.

Tumor Lysates: EBC-1-shMet xenograft tumors were generated and Dox was dosed essentially as described in Example 3, except that Dox was used at 1 mg/ml in 5% sucrose and tumors were allowed to grow to 300-400 mm³ prior to initiation of treatment. Animals were dosed for three days, then sacrificed. Flash frozen EBC-1-shMet-4.12 xenograft tumor samples were placed into 2 mls of cold lysis buffer (PBS+1% TritonX-100+(3×) Phosphatase Cocktail 2 (Sigma cat# P5726)) and Complete Mini EDTA-Free protease inhibitor (Roche #11 836 170 001). Tumors were homogenized with a hand held homogenizer and lysates were incubated on ice for 1 hr with occasional swirling. Lysates were spun down at 10000×G for 10 minutes at 4° C., transferred to a new tube and Her 3 protein was quantified using a BCA assay (Pierce cat# 23225).

### Results

shRNA-mediated knock-down of c-met expression reduced pEGFR levels arid significantly increased HER3 protein levels (FIG. 10A). FACS analysis revealed increased surface HER3 levels after c-met knockdown (FIG. 10B). C-met knockdown in EBC-1shMet-4.12 xenograft tumors resulted in an increase in HER3 protein levels (FIG. 10C).

These data demonstrate that c-met activity can regulate HER3 expression level. Specifically, c-met inhibition resulted in increased HER3 protein levels and decreased pEGFR levels. The decrease in pEGFR after c-met inhibition is likely due to decreased autocrine signaling by EGFR ligands (see FIG. 9) and increased HER3 levels might increase erlotinib sensitivity, as has been demonstrated by others (e.g., Yauch et al. Clin Cancer Res (2005) 11:8686-98). These results suggest that HER3 activity (e.g. signaling through HER2) may increase following inhibition of c-met signaling, and further support the use of combination therapy with c-met and HER3 inhibitors for the treatment of cancer.

### Example 7

### EGFR Pathway Activation Can Restore Cell Proliferation and Viability of Cell in Which C-met Activity is Inhibited

### Materials and Methods

EBC-1 shMet cells were seeded at 5000/well in RPMI 1640 medium (containing 10% Tet-Free FBS from Clontech cat# 631107) in a black-walled 96 well plate, and plates were incubated overnight. Media was replaced with fresh media +/−100 ng/ml dox, and plates were incubated for 48 hours. EGFR ligands were then added to final concentrations described below, and plates were incubated for an additional 48 hours then cell number was determined using Cell TiterGlo (Promega #G7570) as described herein: Dox +100 ng/ml HGF; Dox +50 nM TGFα; Dox +5 ng/ml HGF; and Dox +1 nM TGFα.

### Results

Knockdown of c-met expression by shRNA resulted in a significant decrease in cell number, implying a decrease in cell viability and proliferation. EGFR ligands HGF and TGFα were capable of rescuing cell number in a dose-dependent manner, although HGF appeared to rescue cell number somewhat better than TGFα. These results demonstrated that EGFR pathway activation can restore cell proliferation and cell viability in cells in which c-met signaling activity is inhibited. Thus, EGFR (and/or other HER family members) signaling compensated for loss of c-met signaling activity. These results support the use of combination therapy with c-met and EGFR inhibitors, and are consistent with the dramatically increased xenograft tumor efficacy observed when tumors were treated with the combination of EGFR and c-met inhibitors (Example 4).

### Example 8

### Activation of C-met Results in Activation of EGFR, C-met Interacts with EGFR Independently of C-met or EGFR Pathway Activation, and Activation of C-met Attenuated Response to EGFR Inhibitor

### Materials and Methods

Cells: NCI-H596 cells were obtained from the American Type Culture Collection (ATCC) and were maintained in RPMI 1640 supplemented with 10% fetal bovine serum (FBS; Sigma, St. Louis, Mo.), and 2 mM L-glutamine. Cell assay media was changed as described below depending upon the experiment.

Therapeutics and Growth factors: Erlotinib and MetMAb were from Genentech, Inc., as described above. HGF and TGFα were generated at Genentech.

Immunoprecipitations and Immunoblotting: Cells were starved overnight in 0.1% BSA/RPMI prior to stimulation with ligand and/or dosing with compound, as described in the text. HGF and TGF-α ligands were generated in-house. At the time of harvesting, cells were immediately washed once in ice cold PBS followed by lysing in lysis buffer (CST #9803) supplemented with 1 mM of each of the following: Protease Inhibitors (Sigma Cat #P3840), Phosphatase Inhibitors (Sigma Cat # P2850 and P3726), NaF, Na₃V0₄ and PMSF. Samples were placed on a 180° rotator at 4° C., followed by clearing at 14,000 rpm, 20 min 4° C. Protein concentration was estimated using the Bradford Assay.

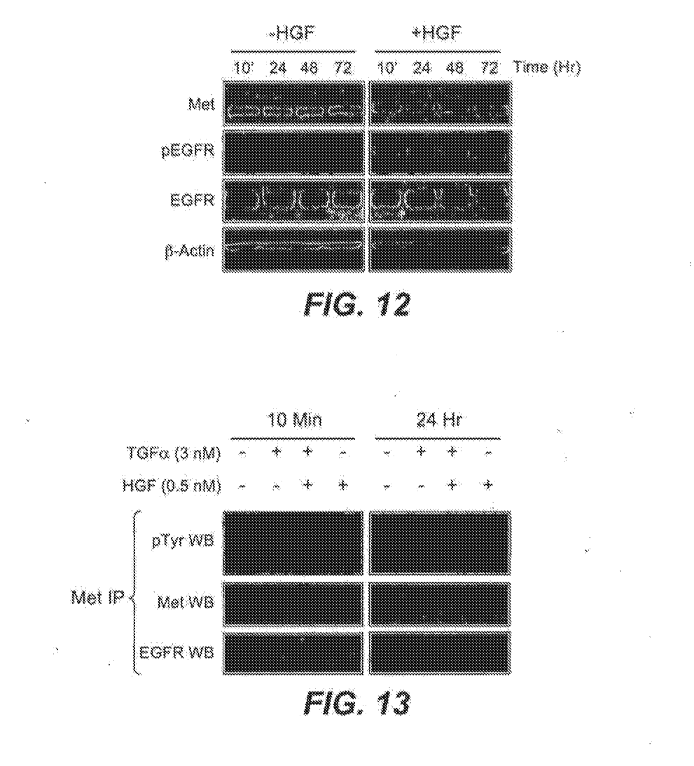

Cell lysates were either directly loaded onto gels (FIG. 12, equivalent lysate concentration of 40 μg/lane) or immunoprecipitated (FIG. 13, equivalent lysate concentration of 1.6 mg/sample). Immunoprecipitation was performed with each of the following antibodies; agarose conjugated cMET: (Santa Cruz Biotechnology Cat #SC-161AC), EGFR (Neomarkers MS-609-P)+Protein A Sepharose Fast Flow Beads. Samples were placed on a 180° rotator, 4° C. overnight, followed by three washes with lysis buffer and subsequently denatured in SDS sample buffer containing beta-mercaptoethanol. Samples were heated for 5 min at 95° C. followed by loading on 4-12% gradient gels and transferring onto nitrocellulose membranes using standard western blotting procedures. Membranes were blocked in 5% milk/TBST for 1 hr, RT and then probed with the following phopho-antibodies over night at 4° C., as indicated in the text: p-c-met: pTyr 4G10 (Upstate Cat# 05-777); pEGFR (Cell Signaling Technologies Cat # 2264). Membranes were stripped with Restore Stripping Buffer (Pierce Cat # 21059) and re-probed with antibodies to total protein: cMET DL-21 (Upstate Biotech Cat #05-238); EGFR (MBL Cat # MI-12-1); beta actin (Santa Cruz Biotechnologies Cat #SC-1616). Secondary antibodies were obtained from Jackson Laboratories. Immunoblots were detected using the ECL Method, as per manufacturer recommendations.

Cell Viability Assays: For cell viability assays, cells were plated in quadruplicate at 1×10³ cells per well in 384-well plates in RPMI containing 0.5% FBS (assay medium) overnight, prior to stimulation with assay medium containing 3 nM TGF-a+/−HGF. Erlotinib was added at multiple concentrations and 72 hours later, cell viability was measured using the Celltiter-Glo Luminescent Cell Viability Assay (Promega, Madison, Wis.).

### Results

Activation of C-met with HGF Results in Activation of EGFR.

Since activation of c-met resulted in the upregulation of numerous EGFR ligands in NCI-H596 cells, we hypothesized that c-met activation results in transactivation of the EGFR pathway. To test this hypothesis, NCI-H596 NSCLC cells were treated with or without HGF in vitro, and cell lysates were analyzed at ten minutes, 24, 48 and 72 hours to examine EGFR pathway activation. Activation of c-met signaling resulted in activation of EGFR signaling (FIG. 12). Induction of pEGFR level was observed as early as ten minutes following HGF stimulation, suggesting that c-met activation directly transactivates EGFR signaling (FIG. 12). Increased levels of pEGFR were observed at the later time points (24, 48, and 72 hours following HGF stimulation) (FIG. 12). The delayed pEGFR activation kinetics are consistent with data showing that c-met activity results in increased expression of EGFR ligands, which could be responsible for delayed (>24 hour) EGFR pathway activation. In this model, activation of EGFR would be predicted to increase at later time points and remain relatively high, consistent with the data shown here.

C-met Interacts with EGFR Independent of C-met or EGFR Pathway Activation Status.

Co-immunoprecipitation experiments (co-IPs) were performed to determine whether c-met and/or EGFR activity might result in physical association of c-met with EGFR. NCI-H596 cells were treated with no ligand, TGFα alone, HGF alone, or TGF-a plus HGF for 10 minutes or 24 hours. Following this treatment, c-met was immunoprecipitated followed by western blotting for either phospho-tyrosine (4G10), EGFR or c-met.

C-met immunoprecipitation pulled down EGFR in the absence of either ligand and at later time points when pc-met and pEGFR levels had dropped, indicating that c-met interacted with EGFR regardless of c-met or EGFR pathway activation status (FIG. 13). The c-met IPs blotted for phospho-tyrosine revealed that EGFR and c-met activation was ligand-dependent and attenuated after 24 hours. Activation of c-met by HGF resulted in co-immunoprecipitation of pEGFR; however pEGFR levels were much lower than pEGFR levels observed when cells were stimulated with TGFα alone or in combination with HGF. Activation of c-met or EGFR by their respective ligands showed that each pathway could be activated independently of one another.

Activation of C-met Attenuated the Response of NCI-H596 Cells to EGFR Inhibitor and Treatment with Anti-c-met Antibody MetMAb Rescued the Response to EGFR Inhibitor.

NCI-H596 cells are sensitive to EGFR inhibitor erlotinib (TARCEVA™) when grown in the presence of TGFα, as demonstrated by reduced cell viability when grown in the presence of erlotinib and TGFα. To determine whether activation of the c-met pathway could change the response of NCI-N596 cells to erlotinib, cells were stimulated with TGFα, treated with erlotinib and/or HGF, then cell viability was assayed.

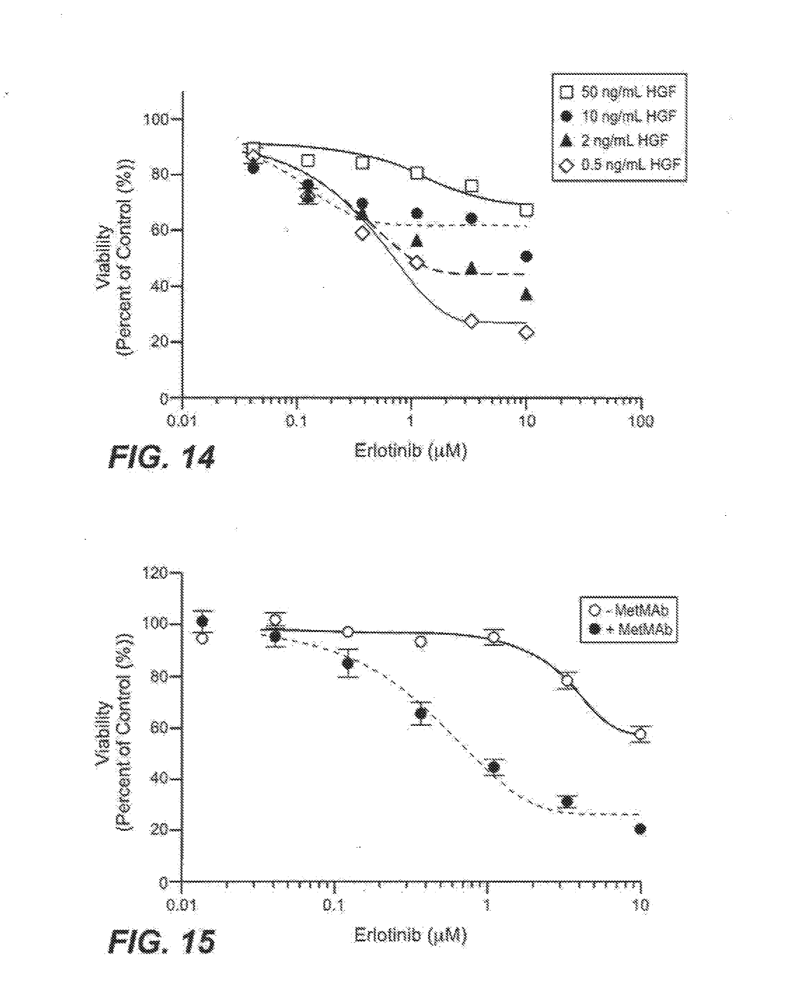

Low levels of HGF showed modest effects upon cell sensitivity to erlotinib; however sensitivity to erlotinib was dramatically reduced in a dose-dependent manner as HGF concentrations increased (FIG. 14), as revealed by increased cell viability under these conditions. These data indicate that HGF activation of the Met pathway is sufficient to attenuate the response of NCI-H596 cells to erlotinib.

To determine whether the combination of c-met inhibitors and EGFR inhibitors reduced cell viability of cell lines that are co-activated by HGF and TGFα, NCI-H596 cell viability assays were performed in the presence of HGF, TGFα, and varying doses of erlotinib and/or c-met antagonist antibody MetMAb (1 uM).

Presence of HGF attenuated response of NCI-H596 cells to erlotinib (FIG. 15). Inhibition of the c-met pathway by MetMAb dramatically restored erlotinib sensitivity (FIG. 15), thus suggesting that treatment with c-met and EGFR inhibitors can have combination effects impacting cell viability in the NCI-H596 cell line.

Taken together, these studies support the hypothesis that activation of the c-met pathway directly activated the EGFR pathway, both through induction of EGFR ligand expression as well as through direct interaction between c-met and EGFR. These results are consistent with dramatically increased xenograft tumor efficacy observed when tumors were treated with the combination of EGFR and c-met inhibitors (Example 4).

### Example 9

### Combination Treatment with C-met Antagonist and EGFR Antagonist Resulted in Better Inhibition of Proliferation and Survival Signaling Pathways in NCI-H596 Xenograft Tumors

### Materials and Methods

NCI-H596 hu-HGF-Tg-SCID xenograft tumors: NCI-H596 xenografts were established in hu-HGF-Tg-SCID mice as described in Example 4. Tumors were allowed to grow to 200-300 mm³ prior to treatment. Dosing was performed as described in Table 8. Briefly, MetMAb (30 mg/kg) or MetMAb buffer was dosed at time zero hours (0 hr) and methylcellulose tween vehicle (MCT) or erlotinib (150 mg/kg) was dosed at time 18 hours (18 hr). Mice were euthanized and tumors and plasma collected at time 24 hours (24 hr). Tumors were snap frozen in liquid nitrogen and then kept at −70° C. until they were processed for immunoprecipitation and immunoblotting.

TABLE 8
Study Design

| | | | | | | | Dose |
|---|---|---|---|---|---|---|---|
| | | Test | | | Dose. | Dose Conc. | Volume |
| Group | No./Sex | Material | Route | Dose Frequency | (mg/kg) | (mg/ml) | (μl) |
| 1 | 5/F | Vehicles: | PO; IP | Once (MCT at 6 hours | 0 | 0 | 100 (ea.) |
| | | MCT; | | prior to tumor harvest, | | | |
| | | MetMAb | | MetMAb buffer 24 | | | |
| | | buffer | | hours prior | | | |
| 2 | 5/F | MetMAb | PO; IP | Once (MCT at 6 hours | 30 | 6 | 100 (ea.) |
| | | | | prior to tumor harvest, | | | |
| | | | | MetMAb 24 hours | | | |
| | | | | prior | | | |
| 3 | 5/F | Erlotinib | PO; IP | Once (erlotinib at 6 | 150 | 37.5 | 100 (ea.) |
| | | | | hours prior to tumor | | | |
| | | | | harvest, MetMAb | | | |
| | | | | buffer 24 hours prior | | | |
| 4 | 5/F | Erlotinib + | PO; IP | Once (erlotinib at 6 | 150; 30 | 37.5; 6 | 100 (ea.) |
| | | MetMAb | | hours prior to tumor | | | |
| | | | | harvest, MetMAb 24 | | | |
| | | | | hours prior | | | |

Immunoprecipitations and Immunoblotting: To process tumors for protein analysis, tumors were first homogenized using a glass dounce with lysis buffer (Cell Signaling Technology, Inc., Danvers, Mass.), supplemented with 1 mM PMSF, additional protease inhibitor cocktail, and phosphatase inhibitor cocktail I and II (Sigma, Inc., St. Louis, Mo.). Lysates were incubated on ice for one hour and then centrifuged at 14,000×g for five minutes and supernatants collected. Protein concentrations were determined using the BCA™ Protein Assay Kit (Pierce, Inc., Rockford, Ill.) and samples were immunoblotted. For immunoprecipitations, 1.5 mg of tumor lysates was used to pull down Met, using the C-28 anti-human c-Met polyclonal antibody (Santa Cruz Biotechnology, Inc., Santa Cruz, Calif.) conjugated agarose beads, or EGFR, using the MI-12-1 antibody (MBL, Inc., Woburn, Mass.) at 4° C. overnight with rotation. The beads were washed three times with lysis buffer at 4° C. followed by resuspension in 1× Novex Tris-Glycine SDS Running Buffer (Invitrogen, Inc., Carlsbad, Calif.) containing 2.5% (w/v) beta-mercaptoethanol. For direct Western blots, 50 μg of tumor lysate was loaded per lane. Samples were then analyzed by SDS-PAGE and immunoblotting. Antibodies used include the mouse anti-human c-Met DL-21, mouse anti-phosphotyrosine mAb 4G10 (both from Upstate Biotechnology/Millepore, Inc., Charlottesville, Va.), anti-Akt, anti-p44/42 MAP kinase (ERK-1/2), anti-phospho-Akt (Ser473), anti-phospho-p44/42 MAP kinase (ERK-1/2) (Thr202/Tyr204), all used according to manufacturer's recommendations (all from Cell Signaling Technology, Inc. (Danvers, Mass.)). Goat-anti-mouse-IRdye800 (Rockland Immunochemicals, Inc., Gilbertsville, Pa.) and goat-anti-rabbit-AlexaFluor680 (Molecular Probes, Inc., Eugene, Oreg.) were used as secondary antibodies. Immunoblots were imaged and phospho-protein levels were quantified and normalized to total protein levels (e.g. pEGFR over total EGFR) using an Odyssey imager (LI-COR Biosciences, Lincoln, Nebr.).

### Results

C-met and EGFR pathway activation were examined in xenograft tumors generated in the NCI-H596 hu-HGF-Tg-SCID mouse xenograft model and treated with EGFR inhibitor, c-met inhibitor and the combination of EGFR and c-met inhibitors. Twenty hu-HGF-Tg-SCID mice were inoculated with NCI-H596 cells and tumors established, as previously described. Once tumors reached sizes between 200-300 mm³, mice were evenly grouped into four groups based upon tumor volume and dosing was begun (Table 8). MetMAb was dosed 24 hours prior to tumor harvest whereas erlotinib was dosed 6 hours prior to harvest. Dosing times were selected based on the relative half-life of each therapeutic agent. At 24 hours, mice were euthanized and tumors were collected and tumors were processed for immunoprecipitations (IPs) and/or immunoblots against phosphorylated and total Met, EGFR, Akt and ERK-1/2.

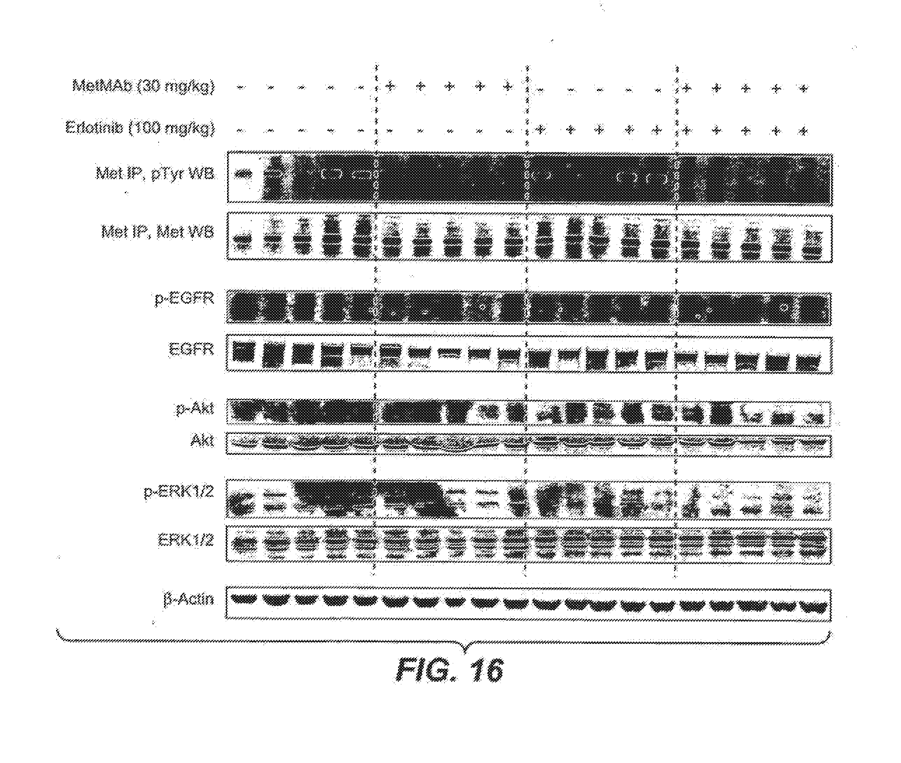

Treatment with MetMAb alone resulted in inhibition of c-met phosphorylation to 12% (+/−3.6%) of vehicle control (FIG. 16), and combined treatment with MetMAb and erlotinib resulted in inhibition of c-met phosphorylation to 6% (+/−3.5%) of vehicle control (FIG. 16) (p=0.039). Treatment with erlotinib alone (FIG. 16) did not reduce c-met phosphorylation. Treatment with erlotinib alone inhibited phosphorylation of EGFR to 16% (+/−7.9%) of vehicle control and combined treatment with erlotinib and MetMAb inhibited phosphorylation of EGFR to 19% (+/−15%) of vehicle control (FIG. 16). Treatment with MetMAb alone also modestly inhibited pEGFR to 62% (+/−21.6%) of vehicle control (p=0.006).

These results demonstrated that MetMAb and erlotinib each effectively inhibit activation of their respective targets and that blockade of c-met can inhibit pEGFR response in the NCI-H596 hu-HGF-Tg-SCID model.

Combined treatment with MetMAb and erlotinib also resulted in more effective inhibition of PI-3K/Akt and the Ras-RAF-MEK-ERK1/2 pathways which are activated downstream of activated Met and EGFR, where the pathways act to activate tumor cell survival and proliferation, respectively, and help drive oncogenesis. Phospho-Akt and phospho-ERK-1/2 was examined in xenograft tumors from animals treated with MetMAb, erlotinib or MetMAb plus erlotinib.

Treatment with MetMAb alone resulted in inhibition of pAkt to 72% (+/−27.9%) of vehicle control and inhibition of pERK-1/2 to 72% (+/−40.3%) of vehicle control (FIG. 15, Table 9). Erlotinib treatment resulted in a more robust inhibition of pAkt to 45% (+/−25.7%) and ERK-1/2 by 39% (+/−8.9%) of vehicle controls, respectively (FIG. 16, Table 9). Treatment with the combination of MetMAb and erlotinib showed improved inhibition of pAkt and pERK-1/2 to 24% (+/−13.8%) of vehicle control and 29% (+/−2.9%) of vehicle control, respectively (FIG. 15, Table 9). These results demonstrated that combined treatment with MetMAb and erlotinib inhibited downstream signaling pathways more effectively than treatment with MetMAb or erlotinib alone.

TABLE 9
Summary of the quantified levels of phospho-proteins*, as percent
of vehicle control, following treatment of NCI-H596 tumor bearing
mice with MetMAb, erlotinib or the combination of MetMAb and
erlotinib. Phospho-protein levels were determined by quantifying
signal intensity of bands by Li-Cor then normalizing to total
protein levels (minus background). Data are represented as a per-
cent of the vehicle control (values represent an average of tumors
from 5 treated different animals each as shown in FIG. 16).

| | Treatment | | | |
|---|---|---|---|---|
| | | | | MetMAb + |
| Protein | Vehicle | MetMAb | Erlotinib | Erlotinib |
| pMet/total Met | 100 (±50.8) | 12 (±3.5) | 157 (±103.4) | 6 (±3.5) |
| pEGFR/total | 100 (±26.6) | 62 (±21.6) | 16 (±7.9) | 19 (±15) |
| EGFR | | | | |
| pAkt/total Akt | 100 (±13.8) | 72 (±27.9) | 45 (±25.7) | 24 (±13.9) |
| pERK1/2/total | 100 (±25.3) | 72 (±40.3) | 39 (±8.9) | 29 (±2.4) |
| ERK1/2 | | | | |

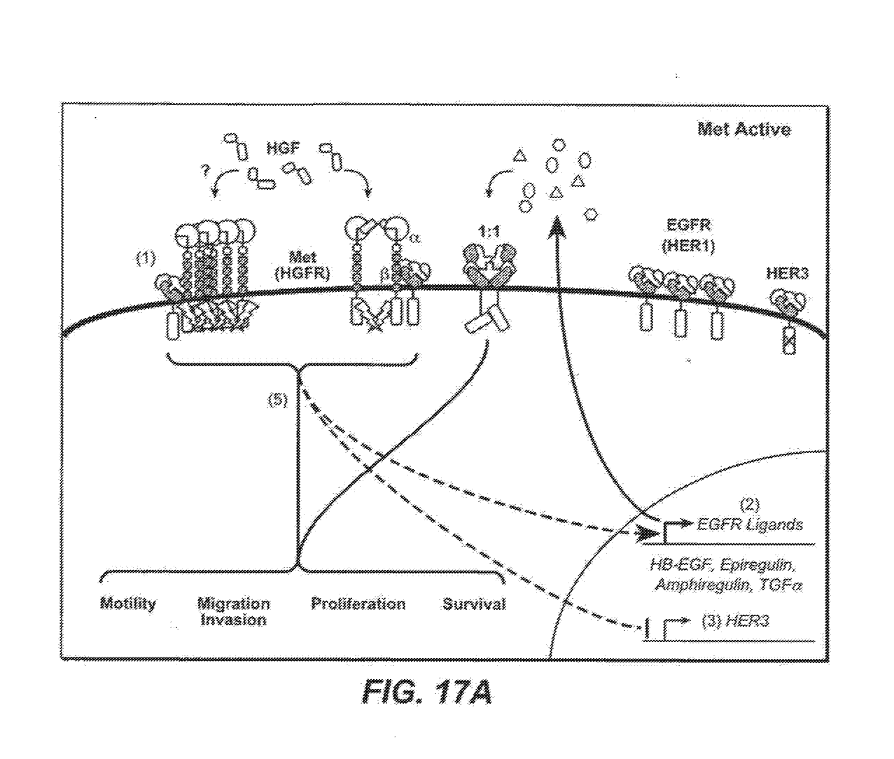

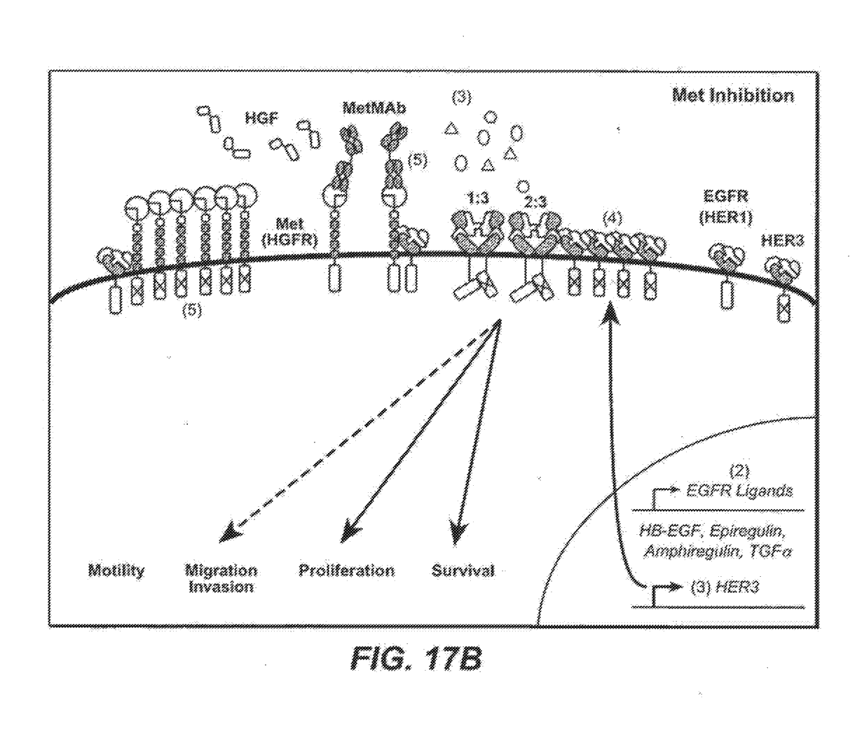

FIG. 17 diagrammatically summarizes some of the findings disclosed herein as follows:

(1) c-met and EGFR were co-expressed in NSCLC cell lines and tumors;

(2) c-met activity positively regulated expression of EGFR ligands and pEGFR;

(3) c-met activity negatively controlled expression of HER3;

(4) TGFα treatment rescued ligand-independent c-met activated cells from c-met inhibitor-mediated loss of viability; and

(5) c-met activation reduced response to erlotinib in vitro and in vivo.

### PARTIAL LIST OF REFERENCES

Bhargava, M, Joseph, A, Knesel, J, Halaban, R, Li, Y, Pang, S. Goldberg, I, Setter, E, Donovan, M A, Zarnegar, R, Michalopoulos, G A, Nakamura, T, Faletto, D. and Rosen, E. (1992). Scatter factor and hepatocyte growth factor: activities, properties, and mechanism. Cell Growth Differ., 3(1); 11-20.

Kong-Beltran, M., Seshagiri, S., Zha, J., Zhu, W., Bhawe, K., Mendoza, N., Holcomb, T., Pujara, K., Stinson, J., Fu, L., Severin, C., Rangell, L., Schwall, R., Amler, L., Wickramasinghe, D., Yauch, R. (2006). Somatic Mutations Lead to an Oncogenic Deletion of Met in Lung Cancer. Cancer Res, 66 (1); 283-289.

Peschard, P., .Fournier, T. M., Lamorte, L, Naujokas, M. A., Band, H., Langton, W. Y., Park, M. (2001). Mutation of the c-Cbl TKB domain binding site on the Met receptor tyrosine kinase converts it into a transforming protein. Mol Cell., 8(5); 995-1004.

Ridgeway, J. B. B., Presta, L. G., Carter, P. (1996). ‘Knobs-into-holes’ engineering of antibody CH3 domains for heavy chain heterodimerization. Protein Engin. 9 (7): 617-621.

Rong, S., Bodescot, M., Blair, D., Dunn, J., Nakamura, T., Mizuno, K., Park, M., Chan, A., Aaronson, S., Vande Woude, G. F. (1992). Tumorigenicity of the met proto-oncogene and the gene for the hepatocyte growth factor. Mol Cell Biol., 12(11); 5152-5158.

Zhang, Y-W., Su, Y., Lanning, N., Gustafson, M., Shinomiya, N., Zhao, P., Cao, B., Tsarfaty, G., Wang, L-M, Hay, R., Vande Woude, G. F. (2005). Enhanced growth of human met-expressing xenografts in a new strain of immunocompromised mice transgenic for human hepatocyte growth factor/scatter factor. Oncogene, 24; 101-106.

Although the foregoing invention has been described in some detail by way of illustration and example for purposes of clarity of understanding, the descriptions and examples should not be construed as limiting the scope of the invention.

What is claimed is:

1. A method of treating cancer in a subject, comprising administering to the subject a therapeutically effective amount of a c-met antagonist and an EGFR antagonist.

2. The method of claim 1, wherein the EGFR antagonist has a general formula I: 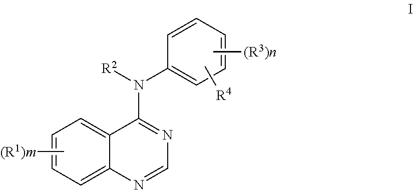
in accordance with U.S. Pat. No. 5,757,498, incorporated herein by reference, wherein:
m is 1, 2, or 3;
each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶-piperazin-1-yl, imidazol-1-yl, 4-pyridon-1-yl, —(C₁-C₄ alkylene)(CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;
or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;
or two R¹ groups are taken together with the carbons to which they are attached to form a 5-8 membered ring that includes 1 or 2 heteroatoms selected from O, S and N;
R² is hydrogen or C₁-C₆ alkyl optionally substituted by 1 to 3 substituents independently selected from halo, C₁-C₄ alkoxy, —NR⁶R⁶, and —SO₂R⁵;
n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and
R⁴ is azido or -(ethynyl)-R¹¹ wherein R¹¹ is hydrogen or C₁-C₆ alkyl optionally substituted by hydroxy, —OR⁶, or —NR⁶R⁶.

3. The method of claim 2, wherein the EGFR antagonist is a compound according to formula I selected from the group consisting of:
(6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine; (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-{6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl]-(5-ethynyl-2-methyl-phenyl)-amine; [6,7-bis-(2-methoxy-ethoxy)-quinazolin-4-yl]-(3-ethynyl-4-fluoro-phenyl)-amine; [6,7-bis-(2-chloro-ethoxy)-quinazolin-4-yl]-(3-ethynyl-phenyl)-amine; [6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7-yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]-ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-[2-(4-methyl-piperazin-1-yl)-ethoxy]-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3 -ethynylphenyl)-amine; 2-[4-(3-ethynyl-phenylamino)-6-(2-methoxy-ethoxy)-quinazolin-7-yloxy]-ethanol; (6,7-dipropoxy-quinazolin-4-yl)-(3-ethynyl-phenyl)-amine; (6,7-diethoxy-quinazolin-4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine; (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine.

4. The method of claim 2, wherein the EGFR antagonist of formula I is N-(3-ethynylphenyl)-6,7-bis(2-methoxyethoxy)-4-quinazolinamine.

5. The method of claim 4, wherein the EGFR antagonist N-(3-ethynylphenyl)-6,7-bis(2-methoxyethoxy)-4-quinazolinamine is in HCl salt form.

6. The method of claim 4, wherein EGFR antagonist N-(3-ethynylphenyl)-6,7-bis(2-methoxyethoxy)-4-quinazolinamine is in a substantially homogeneous crystalline polymorph form that exhibits an X-ray powder diffraction pattern having characteristic peaks expressed in degrees 2-theta at approximately 6.26, 12.48, 13.39, 16.96, 20.20, 21.10, 22.98, 24.46, 25.14 and 26.91.

7. The method of claim 1, wherein the c-met antagonist is an antibody.

8. The method of claim 7, wherein the antibody is a monovalent antibody.

9. The method of claim 7, wherein the antibody is monovalent and comprises a Fc region, wherein the Fc region comprises a first and a second polypeptide, wherein the first polypeptide comprises the Fc sequence depicted in FIG. 7 (SEQ ID NO: 17) and the second polypeptide comprises the sequence depicted in FIG. 8 (SEQ ID NO: 18).

10. The method of claim 7, wherein the antibody comprises (a) a first polypeptide comprising a heavy chain variable domain having the sequence: QVQLQQSGPELVRPGASVKMSCRASGYTFTSYWLHWVKQRPGQGLEWIGMIDPSNSDTRFN PNFKDKATLNVDRSSNTAYMLLSSLTSADSAVYYCATYGSYVSPLDYWGQGTSVTVSS (SEQ ID NO:19), CH1 sequence depicted in FIG. 7 (SEQ ID NO: 16), and the Fc sequence depicted in FIG. 7 (SEQ ID NO: 17); and (b) a second polypeptide comprising a light chain variable domain having the sequence: DIMMSQSPSSLTVSVGEKVTVSCKSSQSLLYTSSQKNYLAWYQQKPGQSPKLLIYWASTRES GVPDRFTGSGSGTDFTLTITSVKADDLAVYYCQQYYAYPWTFGGGTKLEIK (SEQ ID NO:20), and CL1 sequence depicted in FIG. 7 (SEQ ID NO: 8); and (c) a third polypeptide comprising the Fc sequence depicted in FIG. 8 (SEQ ID NO: 18).

11. The method of claim 1, wherein the cancer is selected from the group consisting of breast cancer, colorectal cancer, rectal cancer, non-small cell lung cancer, non-Hodgkins lymphoma, renal cell cancer, prostate cancer, liver cancer, pancreatic cancer, soft-tissue sarcoma, kaposi's sarcoma, carcinoid carcinoma, head and neck cancer, gastric cancer, melanoma, ovarian cancer, mesothelioma, and multiple myeloma

12. The method of claim 11, wherein the cancer is non-small cell lung cancer.

13. The method of claim 1, wherein the cancer is not a EGFR antagonist resistant cancer.

14. The method of claim 1, further comprising administering to the subject a chemotherapeutic agent.

15. The method of claim 1, wherein the EGFR antagonist is 4-(3′-chloro-4′-fluoroanilino)-7-methoxy-6-(3-morpholinopropoxy)quinazoline.

16. The method of claim 1, wherein the EGFR antagonist is N-[3-chloro-4-[(3-fluorophenyl)methoxy]phenyl]-6-[5-[[[2-(methylsulfonyl)ethyl]amino]methyl]-2-furanyl]-4-quinazolinamine.

17. The method of claim 1, wherein the EGFR antagonist is 4-(4-bromo-2-fluoroanilino)-6-methoxy-7-(I-methylpiperidin-4-ylmethoxy)quinazoline.

18. A method for reducing PI3K mediated signaling in a cancer cell comprising contacting the cell with an EGFR antagonist and a c-met antagonist.

19. A method for reducing EGFR-mediated signaling in a cancer cell comprising contacting the cell with an EGFR antagonist and a c-met antagonist.

20. A method for reducing growth and/or proliferation of a cancer cell, or increasing apoptosis of a cancer cell, comprising contacting the cell with an EGFR antagonist and a c-met antagonist.
